SEQUENCE LISTING (122 sequences)

SEQ ID NO: 1 - protein, 209 aa, Homo sapiens
MDSDETGFEHSGLWVSVLAGLLLGACQAHPIPDSSPLLQFGGQVRQRYLYTDDAQQTEAHLEIREDGTVGGAADQSPESLLQLKALKPGVIQILGVKTSRFLCQRPDGALYGSLHFDPEACSFRELLLEDGYNVYQSEAHGLPLHLPGNKSPHRDPAPRGPARFLPLPGLPPALPEPPGILAPQPPDVGSSDPLSMVGPSQGRSPSYAS

SEQ ID NO: 2 - protein, 210 aa, Mus musculus
MEWMRSRVGTLGLWVRLLLAVFLLGVYQAYPIPDSSPLLQFGGQVRQRYLYTDDDQDTEAHLEIREDGTVVGAAHRSPESLLELKALKPGVIQILGVKASRFLCQQPDGALYGSPHFDPEACSFRELLLEDGYNVYQSEAHGLPLRLPQKDSPNQDATSWGPVRFLPMPGLLHEPQDQAGFLPPEPPDVGSSDPLSMVEPLQGRSPSYAS

SEQ ID NO: 3 - protein, 209 aa, Canis lupus familiaris
MGWAEAGFEHLGLWVPVLAVLLLEACRAHPIPDSSPLLQFGGQVRQRYLYTDDAQETEAHLEIRADGTVVGAARQSPESLLELKALKPGVIQILGVKTSRFLCQGPDGTLYGSLHFDPVACSFRELLLEDGYNIYHSETLGLPLRLRPHNSAYRDLAPRGPARFLPLPGLLPAPPEPPGILAPEPPDVGSSDPLSMVGPSQGRSPSYAS

SEQ ID NO: 4 - DNA, 630 bp, Homo sapiens
atggactcggacgagaccgggttcgagcactcaggactgtgggtttctgtgctggctggtcttctgctgggagcctgccaggcacaccccatccctgactccagtcctctcctgcaattcgggggccaagtccggcagcggtacctctacacagatgatgcccagcagacagaagcccacctggagatcagggaggatgggacggtggggggcgctgctgaccagagccccgaaagtctcctgcagctgaaagccttgaagccgggagttattcaaatcttgggagtcaagacatccaggttcctgtgccagcggccagatggggccctgtatggatcgctccactttgaccctgaggcctgcagcttccgggagctgcttcttgaggacggatacaatgtttaccagtccgaagcccacggcctcccgctgcacctgccagggaacaagtccccacaccgggaccctgcaccccgaggaccagctcgcttcctgccactaccaggcctgccccccgcactcccggagccacccggaatcctggccccccagccccccgatgtgggctcctcggaccctctgagcatggtgggaccttcccagggccgaagccccagctacgcttcctga

SEQ ID NO: 5 - DNA, 630 bp, Artificial sequence
  Codon optimized Homo sapiens FGF21 - variant 1
atggattctgatgagacaggcttcgagcacagcggcctgtgggtttcagttctggctggactgctgctgggagcctgtcaggcacaccctattccagatagcagccctctgctgcagttcggcggacaagtgcggcagagatacctgtacaccgacgacgcccagcagacagaagcccacctggaaatcagagaggatggcacagttggcggagccgccgatcagtctcctgaatctctgctccagctgaaggccctgaagcctggcgtgatccagatcctgggcgtgaaaaccagccggttcctgtgccaaagacctgacggcgccctgtatggcagcctgcactttgatcctgaggcctgcagcttcagagagctgctgcttgaggacggctacaacgtgtaccagtctgaggcccatggcctgcctctgcatctgcctggaaacaagagccctcacagagatcccgctcctagaggccctgccagatttctgcctcttcctggattgcctcctgctctgccagagcctcctggaattctggctcctcagcctcctgatgtgggcagctctgatcctctgagcatggtcggacctagccagggcagatctcctagctacgcctcttga

SEQ ID NO: 6 - DNA, 630 bp, Artificial sequence
  Codon optimized Homo sapiens FGF21 - variant 2
atggacagcgatgaaaccgggttcgagcacagcggtctgtgggtgtccgtgctggccggactgctcctgggagcctgtcaggcgcaccccatccctgactcctcgccgctgctgcaattcggcggacaagtccgccagagatacctgtacaccgacgacgcccagcagaccgaagcccacctggaaattcgggaggacgggactgtgggaggcgctgcagatcagtcacccgagtccctcctccaactgaaggccttgaagcccggcgtgattcagatcctgggcgtgaaaacttcccgcttcctttgccaacggccggatggagctctgtacggatccctgcacttcgaccccgaagcctgctcattccgcgagctgctccttgaggacggctataacgtgtaccagtctgaggcccatggactccccctgcatctgcccggcaacaagtcccctcaccgggatcctgccccaagaggcccagctcggtttctgcctctgccgggactgcctccagcgttgcccgaaccccctggtatcctggccccgcaaccacctgacgtcggttcgtcggacccgctgagcatggtcggtccgagccagggaaggtccccgtcctacgcatcctga

SEQ ID NO: 7 - DNA, 630 bp, Artificial sequence
  Codon optimized Homo sapiens FGF21 - variant 3
atggattccgacgaaactggatttgaacattcagggctgtgggtctctgtgctggctggactgctgctgggggcttgtcaggctcaccccatccctgacagctcccctctgctgcagttcggaggacaggtgcggcagagatacctgtataccgacgatgcccagcagacagaggcacacctggagatcagggaggacggaaccgtgggaggagcagccgatcagtctcccgagagcctgctgcagctgaaggccctgaagcctggcgtgatccagatcctgggcgtgaagacatctcggtttctgtgccagcggcccgacggcgccctgtacggctccctgcacttcgatcccgaggcctgttcttttagggagctgctgctggaggacggctacaacgtgtatcagagcgaggcacacggcctgccactgcacctgcctggcaataagtcccctcaccgcgatccagcacccaggggcccagcacgcttcctgcctctgccaggcctgccccctgccctgccagagccacccggcatcctggccccccagcctccagatgtgggctccagcgatcctctgtcaatggtggggccaagtcaggggcggagtccttcatacgcatcataa

SEQ ID NO: 8 - DNA, 633 bp, Mus musculus
atggaatggatgagatctagagttgggaccctgggactgtgggtccgactgctgctggctgtcttcctgctgggggtctaccaagcataccccatccctgactccagccccctcctccagtttgggggtcaagtccggcagaggtacctctacacagatgacgaccaagacactgaagcccacctggagatcagggaggatggaacagtggtaggcgcagcacaccgcagtccagaaagtctcctggagctcaaagccttgaagccaggggtcattcaaatcctgggtgtcaaagcctctaggtttctttgccaacagccagatggagctctctatggatcgcctcactttgatcctgaggcctgcagcttcagagaactgctgctggaggacggttacaatgtgtaccagtctgaagcccatggcctgcccctgcgtctgcctcagaaggactccccaaaccaggatgcaacatcctggggacctgtgcgcttcctgcccatgccaggcctgctccacgagccccaagaccaagcaggattcctgcccccagagcccccagatgtgggctcctctgaccccctgagcatggtagagcctttacagggccgaagccccagctatgcgtcctga

SEQ ID NO: 9 - DNA, 633 bp, Artificial sequence
  Codon optimized Mus musculus FGF21
atggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttga

SEQ ID NO: 10 - DNA, 629 bp, Canis lupus familiaris
atgggctgggccgaggccgggttcgagcacctgggactgtgggtccctgtgctggctgtgcttttgctggaagcctgccgggcacatccgatccctgactccagccccctcctacaatttggaggtcaagttcgacagcggtacctctacaccgacgatgcccaggagacagaggcccacctagagatcagggccgatggcacagggtgggggctgcccgccagagccctgaaagtctcctggagctgaaagccctaaagccaggggtcattcaaatcttgggagtcaaaacatccaggttcctgtgccagggcccagatgggacactatatggctcgctccatttcgaccctgtggcctgcagtttccgagaactgcttcttgaggatgggtacaacatctaccactccgagacccttggtctcccgcttcgcctgcgcccccacaactccgcataccgggacttggcaccccgcgggcctgcccgcttcctgccactgccaggcctgcttccagcacccccagagcctccagggatcctggccccggagcctcctgacgtgggctcctcggaccctctgagcatggtggggccttcacagggccggagtcccagctatgcttcctaa

SEQ ID NO: 11 - DNA, 630 bp, Artificial sequence
  Codon optimized Canis lupus familiaris FGF21
atgggatgggctgaggctggattcgaacacctgggactctgggtgcccgtcctggccgtgctgctcctggaggcttgcagggctcatcccatccctgacagctccccactcctgcagtttggaggacaggtgaggcagcggtacctgtataccgacgatgcccaggagacagaagctcacctggaaattcgggctgatggaacagtggtcggagctgcccgacagtccccagagtctctcctggaactgaaggccctcaaacccggagtgatccagattctgggcgtcaagacttctagattcctgtgccagggaccagacggcaccctgtacggcagcctgcatttcgatcctgtggcctgttcctttcgagagctcctgctcgaagacggctacaacatctatcactctgagaccctgggactcccactgcgactcagacctcataatagtgcctatcgagatctggctcccaggggcccagctaggtttctgccactccccggactgctccctgctccacctgagccacccggcattctggctccagaacctccagacgtgggctctagtgatccactgagtatggtcggcccctcacaggggaggtcacctagctacgccagctga

SEQ ID NO: 12 - DNA, 22 bp, Artificial sequence
  miRT-122a
caaacaccattgtcacactcca

SEQ ID NO: 13 - DNA, 22 bp, Artificial sequence
  miRT-1
ttacatacttctttacattcca

SEQ ID NO: 14 - DNA, 21 bp, Artificial sequence
  miRT-152
ccaagttctgtcatgcactga

SEQ ID NO: 15 - DNA, 23 bp, Artificial sequence
  miRT-199a-5p
gaacaggtagtctgaacactggg

SEQ ID NO: 16 - DNA, 22 bp, Artificial sequence
  miRT-199a-3p
taaccaatgtgcagactactgt

SEQ ID NO: 17 - DNA, 21 bp, Artificial sequence
  miRT-215
gtctgtcaattcataggtcat

SEQ ID NO: 18 - DNA, 21 bp, Artificial sequence
  miRT-192
ggctgtcaattcataggtcag

SEQ ID NO: 19 - DNA, 22 bp, Artificial sequence
  miRT-148a
acaaagttctgtagtgcactga

SEQ ID NO: 20 - DNA, 22 bp, Artificial sequence
  miRT-194
tccacatggagttgctgttaca

SEQ ID NO: 21 - DNA, 22 bp, Artificial sequence
  miRT-133a
cagctggttgaaggggaccaaa

SEQ ID NO: 22 - DNA, 22 bp, Artificial sequence
  miRT-206
ccacacacttccttacattcca

SEQ ID NO: 23 - DNA, 22 bp, Artificial sequence
  miRT-208a-5p
gtataacccgggccaaaagctc

SEQ ID NO: 24 - DNA, 22 bp, Artificial sequence
  miRT-208a-3p
acaagctttttgctcgtcttat

SEQ ID NO: 25 - DNA, 21 bp, Artificial sequence
  miRT-499-5p
aaacatcactgcaagtcttaa

SEQ ID NO: 26 - DNA, 133 bp, Artificial sequence
  chimeric intron composed of introns from human beta-globin and immunoglobulin heavy chain genes
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 27 - DNA, 1671 bp, Artificial sequence
  CAG promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 28 - DNA, 212 bp, Artificial sequence
  CMV promoter
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 29 - DNA, 380 bp, Artificial sequence
  CMV enhancer
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 30 - DNA, 128 bp, Artificial sequence
  Truncated AAV2 5′ ITR
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 31 - DNA, 128 bp, Artificial sequence
  Truncated AAV2 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 32 - DNA, 122 bp, Artificial sequence
  SV40 polyadenylation signal
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 33 - DNA, 449 bp, Artificial sequence
  Rabbit beta-globin polyadenylation signal
gatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 34 - DNA, 592 bp, Artificial sequence
  CMV promoter and CMV enhancer sequence
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagct

SEQ ID NO: 35 - DNA, 7319 bp, Artificial sequence
  pAAV-CAG-moFGF21-dmiRT
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtacccccgacgcggccgctaattctagatcgcgaacaaacaccattgtcacactccagtatacacaaacaccattgtcacactccagatatcacaaacaccattgtcacactccaaggcgaacaaacaccattgtcacactccaaggctattctagatcgcgaattacatacttctttacattccagtatacattacatacttctttacattccagatatcattacatacttctttacattccaaggcgaattacatacttctttacattccaaggctacctgaggcccgggggtacctcttaattaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa

SEQ ID NO: 36 - DNA, 411 bp, Artificial sequence
  mini-CMV promoter
tatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttaactggcttatcgaaattaatacgactcactatagggagacccaagctt

SEQ ID NO: 37 - DNA, 1178 bp, Artificial sequence
  EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 38 - DNA, 623 bp, Artificial sequence
  RSV promoter
catgtttgacagcttatcatcgcagatccgtatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatattcgcgtatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactaaattccgcattgcagagatattgtatttaagtgcctagctcgatacaataaacgccatttgaccattcaccacattggtgtgcacctccaagctgggtaccagct

SEQ ID NO: 39 - DNA, 448 bp, Artificial sequence
  Synapsin 1 promoter
ctgcgctctcaggcacgacacgactcctccgctgcccaccgcagactgaggcagcgctgagtcgccggcgccgcagcgcagatggtcgcgcccgtgcccccctatctcgcgcctcgcgtggtgcggtccggctgggccggcggcggcgcggacgcgaccaaggtggccgggaaggggagtttgcgggggaccggcgagtgacgtcagcgcgccttcagtgctgaggcggcggtggcgcgcgccgccaggcgggggcgaaggcactgtccgcggtgctgaagctggcagtgcgcacgcgcctcgccgcatcctgtttcccctccccctctctgataggggatgcgcaatttggggaatgggggttgggtgcttgtccagtgggtcggggtcggtcgtcaggtaggcacccccaccccgcctcatcctggtcctaaaacccacttgcact

SEQ ID NO: 40 - DNA, 1296 bp, Artificial sequence
  Calcium/calmodulin-dependent protein kinase II (CaMKII) promoter
taacattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagggtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagcgggg

SEQ ID NO: 41 - DNA, 706 bp, Artificial sequence
  Glial fibrillary acidic protein (GFAP) promoter
cgcgtgatctaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggagagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgcggggtctagagtcga

SEQ ID NO: 42 - DNA, 1153 bp, Artificial sequence
  Nestin promoter
gaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaaagatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggagacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctccgaaacgggccctct

SEQ ID NO: 43 - DNA, 438 bp, Artificial sequence
  Homeobox Protein 9 promoter (HB9) promoter
tgaataaatttaagcaggctaattaatatataaactagctcaatttgtcaagttgatttgtattttagttaattgtgaaagtaattaccacatggtcaaattaacagctttctggaaatgaccaagcctgaggttttatttccttcctgggtgaagaaaattcatttttccaagctcttgatgtgatgaataaaagtcataaatctgggtgattggtgcaggcagagtctaaatggcttcatatttcattttaggtttaatagaaatattcatgctctgttttaatgaaattaaattgaagggggatggggctagagtggttagctgatgaattgacaaaaactaatcagctttattgggaaacaggtttaagggcacggacgtgtcaataacgctcagcctgaccccctcttccattagctaggcaggctgattaga

SEQ ID NO: 44 - DNA, 3015 bp, Artificial sequence
  Tyrosine hydroxylase (TH) promoter
ctgctaggggctgcttcccagctactcctcttggctccgtggcttgccttccagcctgtgtgctgtctggagagcctttaaagcctcacttccaccaactagaagtctctccccaaccctgccctgacctcaagtgcacctcttcaaagtcaggtttagcagctgcagctgggggccctgaatcccacccctgctgtcttccttgaagacagaagtgttgggagctgaggatctgggctagagactggctgtatgatccagagaagtagtgtgcttctgggcctcagatttcccttctgtagaacaggtttgtctgaaatggagaggttggtgctcctctgcagggcctagtgggagtcaccatgagtggttaaaagatccagcttgtcttttggtgagctttgagaggaggtaacagggctgagttctggaagcctgaccaagggcagacttaaggggcctcttggagttgttctcatcaaatggggatgggacacagctaaagtgcccagggcttctctgtgcccacagatgctttagatcttggcacagtgtggtctaccagctgtctctctctgtgtatatatatgtatttcatagacagtgtacagtggcctggtttgtgctatcaggctggatatggacagaggcaagagtttgtggcagcagttatctcccaagagagtccaaagacatcatgttttcaagtttaggccaggtgctacttgagagagctcagacacagacaaaggtctggagagcacatgtcctccacccccacctagcttctgttgcaagcacctccagccgagacaagagaacgaattaaaaagcaatatttgtgtcagtgtaagacatttgccgaaaggttaaatccacattcgtgttgctgcagagcagccccctatgcaggatttgttagatacagctccgtcctaccctgtgccagctgagcaaacgccaggctgggtggggtggaacccagcctgggtttgcctcaccctgcaatccccccagcaccctctaaaggaggaccctgtggtgggcatgcagacctagggactgggcatagataacctttgggtttgggcaacagcccccactcctcaggattgaaggctaaggtgcagccagctctgccttcatggtgggaatgtctccacgtgacccctttctgggctgtggagaacactcagagaagagtcctgggatgccaggcaggccagggatgtgctgggcatgttgagacaggagtgggctaagccagcagagttgctgacccaggaagagttcagaaaggggcatggaacatggggaggggtccatagtgagagagagcaggcagtgcagagtaaatagtccctgagctgggggttatgggatttgcaggagcttgctcagagaaggcagaggagagatgctgcgccaagctgggtatcacagagcctcagactcctggaacaggaactgtgggggtcaggtcagcaggggaggttagggagtgttccctttgtactgacttagcatttatcctgcttctaggggggaaggggggccagtgggggatgcacagcaaggcagtgatgtggcaggcagcctgcgggagctcctggttcctggtgtgaaaaagctgggaaggaagagggctgggtctggtaagtacagcaggcagttggctcctgagagtccaagccctgtctagagggtggagtgagatttcagagggagagctaaacggggtgggggctggggagtccaggcttctggctcctgctaatactcagtgtgctgggtcctcagaacctcagggtggccattttcagggtgagagctctgtcctttggcacttctgcagactccagtatccagaggaataaagatggtactcttcctcagttcccttagtgagaggacacctttctctgaagggcttgggcagttgtcctgaaccattgcctgaaggaaggacttgactccagggacatagaatgggctcagcataagtcccctgtagtagagaaaggtcccctctctggtctccttagagatcctgtttccttggctgaggaagctagggtggatctttgtgtaagtgggtgtggatgctcactggaaatcaaaaggccccttggtgttagaccttggggtgccatgggagagttgatcactgagtgcgcccttacatgggggccagctgagaatggggctgcctctagctcgagaccatgatgcagggagtgagtgggggagttcaggatactcttaactaaagcagaggtctgtccccccagggaggggaggtcagaagaccctagggagatgccaaaggctagggttggcaccatgttgcaggctgtgtcttcaaggagatgataatcagaggaatcgaacctgcaaaagtgggccagtcttagatacactatagaggaataatcttctgaaacattctgtgtctcataggacctgcctgaggacccagccccagtgccagcacatacactggggcagtgagtagatagtatactttgttacatgggctggggggacatggcctgtgccctggaggggacttgaagacatccaaaaagctagtgagagggctcctagatttatttgtctccaagggctatatatagccttcctaacatgaacccttgggtaatccagcatgggcgctcccatatgccctggtttgattagagagctctagatgtctcctgtcccagaacaccagccagcccctgtcttcatgtcgtgtctagggcggagggtgattcagaggcaggtgcctgcgacagtggatgcaattagatctaatgggacggaggcctctctcgtccgtcgccctcgctctgtgcccacccccgcctccctcaggcacagcaggcgtggagaggatgcgcaggaggtaggaggtgggggacccagaggggctttgacgtcagcctggcctttaagaggccgcctgcctggcaagggccgtggagacagaactcgggaccaccagcttgcact

SEQ ID NO: 45 - DNA, 1353 bp, Artificial sequence
  Myelin basic protein (MBP) promoter
caccgtggctttaacacttagagaaaatgcatcccctctaatcaataagtcatcgacagtgggtagatggaggaacggcagtgcgtagtaggatgcgtgcaagcatagtctcgtgcatgggtgcatagatcgctgggcaggtggacaaggtgggggtggataaagaagtgggtagatgattgatgttaggtaaatatcactgggtggacagatgggtggtaggtggatggatggttagaatagtcagaagagggatggattgataaggtgaacagatgataaatgggtgatagactggaagggttgtcaaaagaggataagggaagtgtgagctagccgtatttctaaggtcagtaatagagttgggagaagaggttaagttacatccatttaaacctcacacgaagctgagagggaatggacttgctgccgttggtgaggaaagcgttgcatttcccgtgtgcttggttgtgaagtgctcaggtcccacatgaagcagtcaggttactgcggcttacagaggagccagatccaaatgccccgagtaagcacgtccccgagccagaggcctccagcggaatccgggagagggattgctcagtgccctgcttccctggactgtaagctgcagaaagatgtgggaagtcctgttctccactgagaacactaaaagcaccttttgtcaaacgaccgcttcacatctggggcttgtgcactggtggccttttaaaccagagacaacccacaagatacctaacctgcggggctctctggtacagtgagcaactcaggaaatgctttggcttgattgctgtgggctctcaggccatcgccctctggagtggttcttttaatgagaacctgaagattggcccctgagccatgtataccaagcaagctcaatccaggttagctccctctggttggggcaagctaacgtgctccttgggccccgcgcgtaactgtgcgttttataggagacagctagttcaagaccccaggaagaaagcggctttgtccccctctaggcctcgtacaggcccacattcatatctcattgttgttgcaggggaggcagatgcgatccagaacaatgggacctcggctgaggacacggcggtgacagactccaagcacacagcagacccaaagaataactggcaaggcgcccacccagctgacccagggaaccgcccccacttgatccgcctcttttcccgagatgccccgggaagggaggacaacaccttcaaagacaggccctcagagtccgacgagcttcagaccatccaagaagatcccacagcagcttccgaagaattctgcagtcgacggtaccgcgggcccgggatc

SEQ ID NO: 46 - DNA, 7030 bp, Artificial sequence
  pAAV-CAG-moFGF21
agtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttccactgaggcccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctactcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgattaattcgagcgaacgcgtcgagtcgctcggtacgatttaaattgaattggcctcgagcgcaagcttgagctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtacccccgacgcggcctaactggcctcatgggccttccgctcactgcccgctttccagtcgggaaacctgtcgtgccagtcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagctgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaa

SEQ ID NO: 47 - DNA, 20 bp, Artificial sequence
  moFgf21-Fw
cctaaccaggacgccacaag

SEQ ID NO: 48 - DNA, 20 bp, Artificial sequence
  moFgf21-Rv
gttccaccatgctcagaggg

SEQ ID NO: 49 - DNA, 22 bp, Artificial sequence
  Gfap-Fw
acagactttctccaacctccag

SEQ ID NO: 50 - DNA, 20 bp, Artificial sequence
  Gfap-Rv
ccttctgacacggatttggt

SEQ ID NO: 51 - DNA, 22 bp, Artificial sequence
  S100b-Fw
aacaacgagctctctcacttcc

SEQ ID NO: 52 - DNA, 20 bp, Artificial sequence
  S100b-Rv
cgtctccatcactttgtcca

SEQ ID NO: 53 - DNA, 20 bp, Artificial sequence
  Aif1-Fw
tgagccaaagcagggatttg

SEQ ID NO: 54 - DNA, 20 bp, Artificial sequence
  Aif1-Rv
tcaagtttggacggcagatc

SEQ ID NO: 55 - DNA, 20 bp, Artificial sequence
  Nfkb-Fw
gaccactgctcaggtccact

SEQ ID NO: 56 - DNA, 20 bp, Artificial sequence
  Nfkb-Rv
tgtcactatcccggagttca

SEQ ID NO: 57 - DNA, 20 bp, Artificial sequence
  Il1b-Fw
atgaagggctgcttccaaac

SEQ ID NO: 58 - DNA, 20 bp, Artificial sequence
  Il1b-Rv
atgtgctgctgcgagatttg

SEQ ID NO: 59 - DNA, 20 bp, Artificial sequence
  Il6-Fw
tcgctcagggtcacaagaaa

SEQ ID NO: 60 - DNA, 22 bp, Artificial sequence
  Il6-Rv
catcagaggcaaggaggaaaac

SEQ ID NO: 61 - DNA, 20 bp, Artificial sequence
  Ucp1-Fw
ggcctctacgactcagtcca

SEQ ID NO: 62 - DNA, 20 bp, Artificial sequence
  Ucp1-Rv
taagccggctgagatcttgt

SEQ ID NO: 63 - DNA, 5073 bp, Artificial sequence
  pAAV-CMV-moFGF21
ggggctagcgccaccatggaatggatgagaagcagagtgggcaccctgggcctgtgggtgcgactgctgctggctgtgtttctgctgggcgtgtaccaggcctaccccatccctgactctagccccctgctgcagtttggcggacaagtgcggcagagatacctgtacaccgacgacgaccaggacaccgaggcccacctggaaatccgcgaggatggcacagtcgtgggcgctgctcacagaagccctgagagcctgctggaactgaaggccctgaagcccggcgtgatccagatcctgggcgtgaaggccagcagattcctgtgccagcagcctgacggcgccctgtacggctctcctcacttcgatcctgaggcctgcagcttcagagagctgctgctggaggacggctacaacgtgtaccagtctgaggcccacggcctgcccctgagactgcctcagaaggacagccctaaccaggacgccacaagctggggacctgtgcggttcctgcctatgcctggactgctgcacgagccccaggatcaggctggctttctgcctcctgagcctccagacgtgggcagcagcgaccctctgagcatggtggaacctctgcagggcagaagccccagctacgcctcttgagaatgcgggcccggtaccccctcgacggtaccagcgctgtcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgattaggatcttcctagagcatggctacctagacatggctcgacagatcagcgctcatgctctggaagatctcgatttaaatgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcgatatctgtagttaatgattaacccgccatgctacttatctacagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactaggctagctattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactatagaatacgactcactatagggagacgctagcgtcga

SEQ ID NO: 64 - DNA, 397 bp, Artificial sequence
  hAAT promoter
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 65 - DNA, 154 bp, Artificial sequence
  Hepatocyte control region (HCR) enhancer from apolipoprotein E
cagagaggtctctgacctctgccccagctccaaggtcagcaggcagggagggctgtgtgtttgctgtttgctgcttgcaatgtttgcccattttagggacatgagtaggctgaagtttgttcagtgtggacttcagaggcagcacacaaacagc

SEQ ID NO: 66 - DNA, 651 bp, Artificial sequence
  mini/aP2 promoter
gattaacccgccatgctacttatctactcgacattgattattgactaggggaattccagcaggaatcaggtagctggagaatcgcacagagccatgcgattcttggcaagccatgcgacaaaggcagaaatgcacatttcacccagagagaagggattgatgtcagcaggaagtcaccacccagagagcaaatggagttcccagatgcctgacatttgccttcttactggatcagagttcactagtggaagtgtcacagcccaaacactcccccaaagctcagcccttccttgccttgtaacaatcaagccgctcctggatgaactgctccgccctctgtctctttggcagggttggagcccactgtggcctgagcgacttctatggctcccttttctgtgattttcatggtttctgagctcttttcccccgctttatgattttctctttttgtctctctcttgctaaacctccttcgtatatatgccctctcaggtttcatttctgaatcatctactgtgaactattcccattgtttgccagaagccccctggttcttccttctagacaccaggcaaggggcaggaggtaagaggcaggagtccataaaacagccctgagagcctgctgggtcagtgcctgctgtcagaa

SEQ ID NO: 67 - DNA, 722 bp, Artificial sequence
  mini/UCP1
gacgtcacagtgggtcagtcacccttgatcacactgcaccagtcttcacctttccacgcttcctgccagagcatgaatcaggctctctggggataccggcctcacccctactgaggcaaactttctcccacttctcagaggctctgagggcagcaaggtcagccctttctttggaatctagaaccactccctgtcttgagctgacatcacagggcaggcagatgcagcagggaagggcctgggactgggacgttcatcctacaagaaagctgtggaacttttcagcaacatctcagaaatcagatcgcacttattcaaaggagccaggccctgctctgcgccctggtggaggctcctcatgtgaagagtgacaaaaggcaccatgttgtggatacggggcgaagcccctccggtgtgtcctccaggcatcatcaggaactagtgccaaagcagaggtgctggccagggctttgggagtgacgcgcgtctgggaggcttgtgcgcccagggcacgcccctgccgattcccactagcaggtcttgggggacctgggccggctctgcccctcctccagcaatcgggctataaagctcttccaagtcagggcgcagaagtgccgggcgatccgggcttaaagagcgagaggaagggacgctcacctttgagctcctccacaaatagccctggtggctgccacagaagttcgaagttgagagttcgg

SEQ ID NO: 68 - DNA, 326 bp, Artificial sequence
  C5-12 promoter
cggccgtccgccttcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgcca

SEQ ID NO: 69 - DNA, 20 bp, Artificial sequence
  Ccl19-Fw
gcgggctcactggggcacac

SEQ ID NO: 70 - DNA, 21 bp, Artificial sequence
  Ccl19-Rv
tgggaaggtccagagaaccag

SEQ ID NO: 71 - DNA, 23 bp, Artificial sequence
  Ppargc1a-Fw
tttggccgacgacacgacttttc

SEQ ID NO: 72 - DNA, 20 bp, Artificial sequence
  Ppargc1a-Rv
ttgtgttgggcgagagaaag

SEQ ID NO: 73 - DNA, 20 bp, Artificial sequence
  Ppargc1b-Fw
agaagcgctttgaggtgttc

SEQ ID NO: 74 - DNA, 22 bp, Artificial sequence
  Ppargc1b-Rv
ggtgataaaaccgtgcttctgg

SEQ ID NO: 75 - DNA, 20 bp, Artificial sequence
  Atp5f1a-Fw
tctcggccagagactaggac

SEQ ID NO: 76 - DNA, 20 bp, Artificial sequence
  Atp5f1a-Rv
gcacttgcaccaatgaattt

SEQ ID NO: 77 - DNA, 20 bp, Artificial sequence
  Mt-co1-Fw
atgagcaaaagcccacttcg

SEQ ID NO: 78 - DNA, 20 bp, Artificial sequence
  Mt-co1-Rv
accgtggagatttggtccag

SEQ ID NO: 79 - DNA, 19 bp, Artificial sequence
  Cox6-Fw
agtccctctgtcccgtgtc

SEQ ID NO: 80 - DNA, 20 bp, Artificial sequence
  Cox6-Rv
atatgctgaggtcccccttt

SEQ ID NO: 81 - DNA, 19 bp, Artificial sequence
  Cox5a-Fw
ctcgtcagcctcagccagt

SEQ ID NO: 82 - DNA, 20 bp, Artificial sequence
  Cox5a-Rv
tagcagcgaatggaacagac

SEQ ID NO: 83 - DNA, 21 bp, Artificial sequence
  Sod1-Fw
tacacaaggctgtaccagtgc

SEQ ID NO: 84 - DNA, 20 bp, Artificial sequence
  Sod1-Rv
tttccagcagtcacattgcc

SEQ ID NO: 85 - DNA, 20 bp, Artificial sequence
  Nrf2-Fw
agtcgcttgccctggatatc

SEQ ID NO: 86 - DNA, 20 bp, Artificial sequence
  Nrf2-Rv
tgccaaacttgctccatgtc

SEQ ID NO: 87 - DNA, 20 bp, Artificial sequence
  Cat-Fw
tgtgcatgcatgacaaccag

SEQ ID NO: 88 - DNA, 20 bp, Artificial sequence
  Cat-Rv
gcactgttgaagcgtttcac

SEQ ID NO: 89 - DNA, 18 bp, Artificial sequence
  Gapdh-Fw
ccttccgtgttcctaccc

SEQ ID NO: 90 - DNA, 20 bp, Artificial sequence
  Gapdh-Rv
caacctggtcctcactgtag

SEQ ID NO: 91 - DNA, 20 bp, Artificial sequence
  HkI-Fw
acggtcaaaatgctgccttc

SEQ ID NO: 92 - DNA, 20 bp, Artificial sequence
  HkI-Rv
aatcgttcctccgagatcca

SEQ ID NO: 93 - DNA, 20 bp, Artificial sequence
  Pfkp-Fw
tgtgtctgaaggagcaatcg

SEQ ID NO: 94 - DNA, 20 bp, Artificial sequence
  Pfkp-Rv
ggccaaaatcctgtcaaatg

SEQ ID NO: 95 - DNA, 20 bp, Artificial sequence
  Gpd1-Fw
agacacccaactttcgcatc

SEQ ID NO: 96 - DNA, 20 bp, Artificial sequence
  Gpd1-Rv
tattcttcaaggccccacag

SEQ ID NO: 97 - DNA, 20 bp, Artificial sequence
  Gpd2-Fw
ttgccttgggagaagatgac

SEQ ID NO: 98 - DNA, 20 bp, Artificial sequence
  Gpd2-Rv
agttccgcacttcattcagg

SEQ ID NO: 99 - DNA, 20 bp, Artificial sequence
  Pkm-Fw
gctttgcatctgatcccatt

SEQ ID NO: 100 - DNA, 20 bp, Artificial sequence
  Pkm-Rv
agtccagccacaggatgttc

SEQ ID NO: 101 - DNA, 20 bp, Artificial sequence
  Syp-Fw
acatggacgtggtgaatcag

SEQ ID NO: 102 - DNA, 20 bp, Artificial sequence
  Syp-Rv
aagatggcaaagacccactg

SEQ ID NO: 103 - DNA, 20 bp, Artificial sequence
  Gria1-Fw
ccatgctggttgccttaatc

SEQ ID NO: 104 - DNA, 20 bp, Artificial sequence
  Gria1-Rv
ccgtatggcttcattgatgg

SEQ ID NO: 105 - DNA, 20 bp, Artificial sequence
  Gria2-Fw
aagggcgtgtaatccttgac

SEQ ID NO: 106 - DNA, 20 bp, Artificial sequence
  Gria2-Rv
tttcagcaggtctccatcag

SEQ ID NO: 107 - DNA, 20 bp, Artificial sequence
  Grin1-Fw
tgactacccgaatgtccatc

SEQ ID NO: 108 - DNA, 20 bp, Artificial sequence
  Grin1-Rv
ttgtagacgcgcatcatctc

SEQ ID NO: 109 - DNA, 20 bp, Artificial sequence
  Grin2a-Fw
tgtgaagaagtgctgcaagg

SEQ ID NO: 110 - DNA, 20 bp, Artificial sequence
  Grin2a-Rv
cgcctatcattccattccac

SEQ ID NO: 111 - DNA, 20 bp, Artificial sequence
  Grin2b-Fw
ttggtgaggtggtcatgaag

SEQ ID NO: 112 - DNA, 20 bp, Artificial sequence
  Grin2b-Rv
tgcgtgataccatgacactg

SEQ ID NO: 113 - DNA, 20 bp, Artificial sequence
  Sqstm1-Fw
tgctggcggctttacatttg

SEQ ID NO: 114 - DNA, 22 bp, Artificial sequence
  Sqstm1-Rv
cagaagcagagaaggaaaagcc

SEQ ID NO: 115 - DNA, 20 bp, Artificial sequence
  Atg5-Fw
agatggacagctgcacacac

SEQ ID NO: 116 - DNA, 20 bp, Artificial sequence
  Atg5-Rv
ttggctctatcccgtgaatc

SEQ ID NO: 117 - DNA, 18 bp, Artificial sequence
  Atf4-Fw
atgatggcttggccagtg

SEQ ID NO: 118 - DNA, 22 bp, Artificial sequence
  Atf4-Rv
ccattttctccaacatccaatc

SEQ ID NO: 119 - DNA, 18 bp, Artificial sequence
  Bip-Fw
ctgaggcgtattgggaag

SEQ ID NO: 120 - DNA, 21 bp, Artificial sequence
  Bip-Rv
tcatgacattcagtccagcaa

SEQ ID NO: 121 - DNA, 20 bp, Artificial sequence
  Cyp46a1-Fw
tcgttgaacgtctccatcag

SEQ ID NO: 122 - DNA, 20 bp, Artificial sequence
  Cyp46a1-Rv
tttggggagagactgtttgg

(19) United States
(12) Patent Application Publication
(10) Pub. No.: US 2023/0201306 A1
(43) Pub. Date: Jun. 29, 2023

(54) Fibroblast growth factor 21 (FGF21) gene therapy for central nervous system disorders

(71) Applicant: Universitat Autònoma de Barcelona, Cerdanyola del Vallès (ES)

(72) Inventors: Maria Fatima Bosch Tubert, Cerdanyola del Valles (ES); Veronica Jiménez Cenzano, Sabadell (ES); Ivet Elias Puigdomenech, Cerdanyola del Vallès (ES); Ignasi Grass Costa, Sabadell (ES); Claudia Jambrina Pallarés, Tarragona (ES); Victor Sacristan Fraile, Montcada i Reixac Barcelona (ES)

(73) Assignee: Universitat Autònoma de Barcelona, Cerdanyola del Vallès (ES)

(21) Appl. No.: 17/999,717

(22) PCT Filed: May 26, 2021
(86) PCT No.: PCT/EP2021/064060

§ 371 Date: Nov. 23, 2022

(30) Foreign Application Priority Data
May 26, 2020 (EP) 20382442.0

(51) Int. Cl. A61K 38/18; C12N 15/86; A61P 25/00

(52) U.S. Cl. CPC A61K 38/1825; C12N 15/86; A61P 25/00; C12N 2750/14143

(57) ABSTRACT

Described herein is a gene construct comprising a nucleotide sequence encoding a fibroblast growth factor 21 (FGF21), for use in the treatment and/or prevention of a central nervous system (CNS) disorder or disease, or a condition associated therewith.

### FIELD

Aspects and embodiments described herein relate to the field of medicine, particularly gene therapy for central nervous system disorders.

### BACKGROUND

Aging is associated with a decline in cognitive function and is a major risk factor for neurodegeneration and dementia (Wyss-Coray, T. Ageing, neurodegeneration and brain rejuvenation. Nature 539, 180-186 (2016)). Neuromusculoskeletal performance, muscular strength and locomotor activity have also been reported to decline with age (Lynch, M. A. (2004). Physiol. Rev. 84, 87-136; Wenz, T. et al. 2009. Proc Natl Acad Sci USA 106 (48), 20405-10; Lhotellier L, Cohen-Salmon C. Physiol Behav 1989; 45:491-493). The most common neurodegenerative diseases, Alzheimer disease (AD) and Parkinson disease (PD), are predominantly observed in elderly individuals, and the risk of these diseases increases with increasing age. One in ten individuals aged ≥65 years has AD and its prevalence continues to increase with age. AD prevalence is expected to double within the next 20 years (Prince M., et al. The global prevalence of dementia: a systematic review and metaanalysis. Alzheimer's Dement 9, e62(2013)). The main clinical features of AD are late-life memory and learning deficits, disorientation, mood swings and behavioural issues (Hou, Y. et al. Ageing as a risk factor for neurodegenerative disease. Nature Reviews Neurology volume 15, pages 565-581(2019)). Diagnostic features of PD include neuromuscular dysfunction that affects movement amplitude and speed, rigidity and/or rest tremor (Hou, Y. et al. Nature Reviews Neurology volume 15, pages 565-581(2019)).

Anxiety and depression disorders are also major public health concerns. Specifically, anxiety disorders are the most common of all mental health problems that affect human beings (Zhang et al., Neuroscience, 196, 203-14 (2011)).

Metabolic disorders (such as diabetes and obesity) are progressive diseases which also cause dementia, depression, anxiety, stroke and Alzheimer's disease (AD) (R. Mayeux, Y. Stern, Cold Spring Harb. Perspect. Med. 2, a006239 (2012); Asato et al, Nihon Shinkei Seishin Yakurigaku Zasshi, 32 (5-6), 251-5 (2012); O. Guillemot-Legris, G. G. Muccioli, Trends Neurosci. 40, 237-253 (2017))(9). Indeed, obese patients are more prone to develop such central disorders than non-obese subjects (A. J. Bruce-Keller, J. N. Keller, C. D. Morrison, Biochim. Biophys. Acta. 1792, 395-400 (2009). T2DM nearly doubles the risk for Alzheimer's Disease (AD) (Ohara et al., 2011). Similarly, it is well recognized that the prevalence of anxiety and depression is higher in diabetic and obesity patients than in the general population (Asato et al, Nihon Shinkei Seishin Yakurigaku Zasshi, 32 (5-6), 251-5 (2012)). The combined overall relative risk for dementia, including clinical diagnoses of both AD and vascular dementia, is 73% higher in people with T2DM than in those without (Gudala et al., J. Diabetes Investig. 2013, 27; 4(6):640-50). Likewise, AD patients experience brain insulin resistance and hyperinsulinemia (Biessels and Reagan, 2015, Nat Rev Neurosci. 2015 16(11):660-71; Stanley et al, 2016, J Exp Med. 25; 213(8):1375-85). This suggests that insulin resistance promotes cognitive impairments leading to AD, and that insulin-deprived brains are susceptible to the development of AD.

Several studies in mice have addressed the link between diet-induced obesity and insulin resistance and cognitive impairment and have shown that, given sufficient exposure to High Fat Diet (HFD), insulin resistant-obese rodents display dramatic changes in their behaviour with impaired spatial learning ability, spatial memory and recognition memory as well as increased anxiety, anhedonia, and depression-like symptoms (Guillemot-Legris, O. et al., 2017, Trends Neurosci. 40, 237-253).

Obesity also impairs neuromuscular function, locomotor capacity and coordination both in mice and humans (Garland T, et al. J Exp Biol 2011; 214: 206-229; Seebacher, F. et al., International Journal of Obesity volume 41, pages 1271-1278(2017); Pérez L M, et al. J Physiol (Lond) 2016; 594: 3187-3207; Zhang, Y., et al. Arch Biochem Biophys, 576, 39-48 (2015))

Few or no effective treatments are available for ageing-related cognitive and neuromuscular decline as well as for neurodegenerative diseases, which tend to progress in an irreversible manner and are associated with large socioeconomic and personal costs. Similarly, anxiety and depression disorders are often resistant to current therapeutic approaches such as anxiolytic or anti-depression drug treatment and cognitive behaviour therapy. Accordingly, novel treatment strategies are required.

Fibroblast growth factor 21 (FGF21), a growth factor predominantly secreted by the liver, but also by adipose tissue and pancreas (Muise, E. S. et al., 2008. Mol. Pharmacol. 74:403-412), is a glucose and lipid metabolism regulator. Moreover, recent reports have also described that FGF21 exerts therapeutic benefit on neurodegeneration, remyelination, cognitive decline, Alzheimer's disease, mood stabilizers and depression (Kuroda, M. et al., 2017. J Clin Invest. 127(9):3496-3509; Sharor, R. A. et al., 2019. J Neurotrauma. 37(1):14-26; Yu, Y. et al., 2015. Pharmacol Biochem Behav. 133:122-31; Wang, X-M. et al., 2016. Exp Cell Res. 346(2):147-56; Wang, Q. et al., 2018. Mol Neurobiol. 55:4702-4717; Sa-nguanmoo P. et al 2016. Hormones and Behavior. 85: 86-95; Sa-nguanmoo P. et al 2018. Biomedicine & Pharmacotherapy. 97:1663-1672; Rühlmann C. et al., 2016. Aging. 8(11):2777-2789; Chen S. et al., 2019. Redox Biol. 22:101133; Amiri M. et al., 2018. Neurotoxicity Research. 34:574-583; Leng, Y. et al., 2015. Molecular Psychiatry. 20, 215-223; Wang, X. et al., Front. Pharmacol., 28 Feb. 2020). However, native FGF21 protein exhibits poor pharmacokinetic characteristics. It has a short half-life, and it is susceptible to in vivo proteolytic degradation and in vitro aggregation (Huang, J. et al., 2013. J Pharmacol Exp Ther. 346(2):270-80; So, W. Y. and Leung, P. S. 2016. Med Res Rev. 36(4):672-704; Zhang, J. and Li, Y. 2015. Front Endocrinol (Lausanne). 6:168). Various molecular engineering approaches have been developed to extend the half-life and to improve the stability and solubility of FGF21. Currently, three engineered FGF21 mimetics (LY2405319, PF-05231023 and BMS-986036) are being tested in humans. Nevertheless, those FGF21 mimetics require multiple administrations, which poses a significant burden to the patients. Moreover, engineered FGF21 mimetics/analogs may exhibit a higher risk of immunogenicity than native FGF21, e.g. patients treated with LY2405319 developed injection site reactions, anti-drug antibodies and a serious hypersensitivity reaction (Gaich, G. et al., 2013. Cell Metab. 18(3):333-40). Injection-site reactions and anti-drug antibodies were also reported in patients treated with PF-05231023 or BMS-986036 (Kim, A. M. et al., 2017. Diabetes Obes Metab. 19(12):1762-1772; Charles, E. D., et al., 2019. Obesity. 27(1):41-49; Sanyal, A. et al., 2019. Lancet. 392(10165):2705-2717).

Therefore, there is still a need for new treatments for neuromuscular and cognitive decline which do not have all the drawbacks of existing treatments

### SUMMARY

An aspect of the invention relates to a gene construct comprising a nucleotide sequence encoding a fibroblast growth factor 21 (FGF21), for use in the treatment and/or prevention of a central nervous system (CNS) disorder or disease, or a condition associated therewith. In some embodiments, a gene construct of the invention is such that the nucleotide sequence encoding FGF21 is operably linked to a ubiquitous promoter, preferably wherein the ubiquitous promoter is selected from the group consisting of a CAG promoter and a CMV promoter. In some embodiments, a gene construct of the invention is such that it comprises at least one target sequence of a microRNA expressed in a tissue where the expression of FGF21 is wanted to be prevented, preferably wherein the at least one target sequence of a microRNA is selected from those target sequences that bind to microRNAs expressed in heart and/or liver of a mammal. In some embodiments, a gene construct of the invention is such that it comprises at least one target sequence of a microRNA expressed in the liver and at least one target sequence of a microRNA expressed in the heart, preferably wherein a target sequence of a microRNA expressed in the heart is selected from SEQ ID NO's: 13 and 21-25 and a target sequence of a microRNA expressed in the liver is selected from SEQ ID NO's: 12 and 14-20, more preferably wherein the gene construct comprises a target sequence of microRNA-122a (SEQ ID NO: 12) and a target sequence of microRNA-1 (SEQ ID NO: 13). In some embodiments, a gene construct of the invention is such that the nucleotide sequence encoding FGF21 is selected from the group consisting of:
(a) a nucleotide sequence encoding a polypeptide represented by an amino acid sequence comprising a sequence that has at least 60% sequence identity or similarity with the amino acid sequence of SEQ ID NO: 1, 2 or 3;
(b) a nucleotide sequence that has at least 60% sequence identity with the nucleotide sequence of SEQ ID NO: 4, 5, 6, 7, 8, 9, 10 or 11; and
(c) a nucleotide sequence the sequence of which differs from the sequence of a nucleotide sequence of (b) due to the degeneracy of the genetic code.

Another aspect of the invention relates to an expression vector comprising a gene construct of the invention, for use in the treatment and/or prevention of a central nervous system (CNS) disorder or disease, or a condition associated therewith. In some embodiments, the expression vector of the invention is a viral vector, preferably selected from the group consisting of adenoviral vectors, adeno-associated viral vectors, retroviral vectors, and lentiviral vectors. In some embodiments, the expression vector of the invention is an adeno-associated viral vector, preferably an adeno-associated viral vector of serotype 1, 2, 3, 4, 5, 6, 7, 8, 9, rh10, rh8, Cb4, rh74, DJ, 2/5, 2/1, 1/2 or Anc80, more preferably an adeno-associated viral vector of serotype 1, 8 or 9.

Another aspect of the invention relates to a pharmaceutical composition comprising a gene construct of the invention and/or an expression vector of the invention, optionally further comprising one or more pharmaceutically acceptable ingredients, for use in the treatment and/or prevention of a central nervous system (CNS) disorder or disease, or a condition associated therewith.

In some embodiments of a gene construct and/or an expression vector and/or a pharmaceutical composition for use according to the invention, the central nervous system (CNS) disorder or disease, or a condition associated therewith, is associated with and/or caused by aging and/or a metabolic disorder or disease, preferably obesity and/or diabetes. In some embodiments of a gene construct and/or an expression vector and/or a pharmaceutical composition for use according to the invention, the central nervous system (CNS) disorder or disease, or a condition associated therewith, is neuroinflammation, neurodegeneration, cognitive decline and/or a disease or condition associated therewith. In some embodiments, the disease or condition associated with neuroinflammation, neurodegeneration and/or cognitive decline is selected from the group consisting of: a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases, preferably selected from the group consisting of Alzheimer's disease, Parkinson's disease, Parkinson-like disease and Huntington's disease, more preferably selected from the group consisting of Alzheimer's disease and Parkinson's disease, most preferably Alzheimer's disease.

In some embodiments of a gene construct and/or an expression vector and/or a pharmaceutical composition for use according to the invention, the central nervous system (CNS) disorder or disease, or a condition associated therewith, is a behavioral disorder, preferably an anxiety disorder or a depressive disorder.

In some embodiments of a gene construct and/or an expression vector and/or a pharmaceutical composition for use according to the invention, the central nervous system (CNS) disorder or disease, or a condition associated therewith is a neuromuscular disorder, preferably the neuromuscular disorder is, or is associated with, declined muscle function, declined muscle strength, declined coordination, declined balance and/or hypoactivity.

Another aspect of the invention relates to a method for improving memory and/or learning in a subject, the method comprising administering to the subject a gene construct and/or an expression vector and/or a pharmaceutical composition of the invention, preferably the subject is an elderly subject and/or a subject diagnosed with a metabolic disorder or disease, preferably diabetes and/or obesity.

Another aspect of the invention relates to a method for improving muscle function, muscle strength, coordination, balance and/or hypoactivity in a subject, the method comprising administering to the subject a gene construct and/or an expression vector and/or a pharmaceutical composition of the invention, preferably the subject is an elderly subject and/or a subject diagnosed with a metabolic disorder or disease, preferably diabetes and/or obesity.

In a further aspect the invention relates to a method of treatment and/or prevention of a central nervous system (CNS) disorder or disease, or a condition associated therewith, comprising administering a gene construct, an expression vector and/or a composition of the invention.

In a further aspect the invention relates to a use of a gene construct, an expression vector or a composition of the invention, for the manufacture of a medicament for the treatment and/or prevention of a central nervous system (CNS) disorder or disease, or a condition associated therewith.

In a further aspect the invention relates to a use of a gene construct, an expression vector or a composition of the invention, for the treatment and/or prevention of a central nervous system (CNS) disorder or disease, or a condition associated therewith.

### DESCRIPTION

The present inventors have developed an improved gene therapy strategy based on FGF21 to counteract central nervous system (CNS) disorders. Particularly, as elaborated in the experimental part, the following unexpected advantages have been found. AAV-mediated FGF21 gene therapy mediates robust overexpression using different administration modes and different types of vectors in several different mouse models. Robust overexpression leads to increased circulating levels of FGF21 and was shown to exert at least the following benefits:
improved coordination, balance, neuromuscular performance, strength and locomotor activity (Examples 1-4, 8, 10, 12 and 13)
enhanced memory and learning (Examples 1, 3, 4, 8, 9, 10, 12 and 13)
decreased neurodegeneration by improving mitochondrial function and diminution of oxidative stress (Examples 1 and 11)
reduced anxiety-like and depression-like behavior (Examples 2-4 and 12)
improved cognitive performance, memory, learning and exploratory capacity (Examples 1, 3, 4, 8, 9, 10, 12 and 13)
decreased neuroinflammation (Examples 5 and 8)

Accordingly, the aspects and embodiments of the present invention as described herein solve at least some of the problems and needs as discussed herein.

Gene Construct

In a first aspect, there is provided a gene construct comprising a nucleotide sequence encoding a fibroblast growth factor 21 (FGF21). In some embodiments, a gene construct as described herein is for use in therapy. In a preferred embodiment, a gene construct as described herein is for use in the treatment and/or prevention of a central nervous system (CNS) disorder or disease, or a condition associated therewith. In a preferred embodiment, a gene construct as described herein is for use in the treatment and/or prevention of a central nervous system (CNS) disorder or disease.

A “gene construct” as described herein has its customary and ordinary meaning as understood by one of skill in the art in view of this disclosure. A “gene construct” can also be called “expression cassette” or “expression construct” and refers to a gene or a group of genes, including a gene that encodes a protein of interest, which is operably linked to a promoter that controls its expression. The part of this application entitled “general information” comprises more detail as to a “gene construct”. “Operably linked” as used herein is further described in the part of this application entitled “general information”.

In some embodiments, a gene construct as described herein is suitable for expression in a mammal. As used herein, “suitable for expression in a mammal” may mean that the gene construct includes one or more regulatory sequences, selected on the basis of the mammalian host cells to be used for expression, that is operatively linked to the nucleotide sequence to be expressed. Preferably, said mammalian host cells to be used for expression are human, murine or canine cells.

A nucleotide sequence encoding an FGF21 present in a gene construct according to the invention may be derived from any FGF21 gene or FGF21 coding sequence, preferably an FGF21 gene or FGF21 coding sequence from human, mouse or dog; or a mutated FGF21 gene or FGF21 coding sequence, preferably from human, mouse or dog; or a codon optimized FGF21 gene or FGF21 coding sequence, preferably from human, mouse or dog.

Accordingly, in some embodiments, a preferred nucleotide sequence encoding an FGF21 encodes a polypeptide represented by an amino acid sequence comprising a sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity or similarity with SEQ ID NO: 1, 2 or 3. SEQ ID NO: 1 represents an amino acid sequence of human FGF21. SEQ ID NO: 2 represents an amino acid sequence of murine FGF21. SEQ ID NO: 3 represents an amino acid sequence of canine FGF21. In some embodiments, a nucleotide sequence encoding an FGF21 present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with any sequence selected from the group consisting of SEQ ID NO's: 4, 5, 6, 7, 8, 9, 10 or 11.

A description of “identity” or “sequence identity” and “similarity” or “sequence similarity” has been provided under the section entitled “general information”.

In some embodiments, a nucleotide sequence encoding a human FGF21 present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 4, 5, 6 or 7. SEQ ID NO: 4 is a nucleotide sequence encoding human FGF21. SEQ ID NO: 5 is a codon optimized nucleotide sequence encoding human FGF21, variant 1. SEQ ID NO: 6 is a codon optimized nucleotide sequence encoding human FGF21, variant 2. SEQ ID NO: 7 is a codon optimized nucleotide sequence encoding human FGF21, variant 3. Variant 1, variant 2 and variant 3 encode for the same human FGF21 protein and were obtained by different algorithms of codon optimization. A description of “codon optimization” has been provided under the section entitled “general information”.

In some embodiments, a nucleotide sequence encoding mouse FGF21 present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 8 or 9. SEQ ID NO: 8 is a nucleotide sequence encoding mouse FGF21. SEQ ID NO: 9 is a codon optimized nucleotide sequence encoding mouse FGF21.

In some embodiments, a nucleotide sequence encoding canine FGF21 present in a gene construct according to the invention has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: 10 or 11. SEQ ID NO: 10 is a nucleotide sequence encoding canine FGF21. SEQ ID NO: 11 is a codon optimized nucleotide sequence encoding canine FGF21.

In some embodiments, there is provided a gene construct as described herein, wherein the nucleotide sequence encoding an FGF21 is selected from the group consisting of:
(a) a nucleotide sequence encoding a polypeptide represented by an amino acid sequence comprising a sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity or similarity with the amino acid sequence of SEQ ID NO: 1, 2 or 3.
(b) a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with the nucleotide sequence of SEQ ID NO: 4, 5, 6, 7, 8, 9, 10 or 11.
(c) a nucleotide sequence the sequence of which differs from the sequence of a nucleotide sequence of (b) due to the degeneracy of the genetic code.

In a preferred embodiment, a nucleotide sequence encoding an FGF21 is a codon-optimized nucleotide sequence, preferably a codon-optimized human sequence, preferably selected from the sequences of SEQ ID NO: 5, 6 and 7.

An FGF21 encoded by the nucleotide sequences described herein exerts at least a detectable level of an activity of an FGF21 as known to a person of skill in the art. An activity of an FGF21 can be to exhibit an anti-obesity and/or an anti-diabetes effect. An activity of an FGF21 can also be to increase insulin sensitivity. This activity could be assessed by methods known to a person of skill in the art, for example by using an insulin tolerance test or a glucose tolerance test. An activity of an FGF21 can also be to decrease neuroinflammation, decrease neurodegeneration, decrease cognitive decline, improve neuromuscular performance, improve behavioral disorders such as depression and depression-like behavior and anxiety and anxiety-like behavior. These activities of an FGF21 could be assessed by methods known to a person of skill in the art, for example by using any of the methods described in the experimental section.

In some embodiments, the nucleotide sequence encoding FGF21 is operably linked to a ubiquitous promoter. A preferred ubiquitous promoter is selected from a CMV promoter and a CAG promoter.

In a preferred embodiment, the ubiquitous promoter is a CAG promoter.

In some embodiments, the nucleotide sequence encoding FGF21 is operably linked at least one target sequence of a microRNA expressed in a tissue where the expression of FGF21 is wanted to be prevented. In some embodiments, the nucleotide sequence encoding FGF21 is operably linked to a ubiquitous promoter and at least one target sequence of a microRNA expressed in a tissue where the expression of FGF21 is wanted to be prevented.

A description of “ubiquitous promoter”, “operably linked” and “microRNA” has been provided under the section entitled “general information”. A “target sequence of a microRNA expressed in a tissue” or “target sequence binding to a microRNA expressed in a tissue” or “binding site of a microRNA expressed in a tissue” as used herein refers to a nucleotide sequence which is complementary or partially complementary to at least a portion of a microRNA expressed in said tissue, as described elsewhere herein.

In some embodiments, the at least one target sequence of a microRNA is selected from those target sequences that bind to microRNAs expressed in heart and/or liver of a mammal.

In some embodiments, the nucleotide sequence encoding FGF21 is operably linked to at least one target sequence of a microRNA expressed in the liver and at least one target sequence of a microRNA expressed in the heart. In some embodiments, the nucleotide sequence encoding FGF21 is operably linked to a ubiquitous promoter and at least one target sequence of a microRNA expressed in the liver and at least one target sequence of a microRNA expressed in the heart. A target sequence of a microRNA expressed in the heart is preferably selected from SEQ ID NO's: 13 and 21-25, more preferably SEQ ID NO: 12 (micro-RNA-122a) and a target sequence of a microRNA expressed in the liver is preferably selected from SEQ ID NO's: 12 and 14-20, more preferably SEQ ID NO: 13 (microRNA-1).

A “target sequence of a microRNA expressed in the liver” or “target sequence binding to a microRNA expressed in the liver” or “binding site of a microRNA expressed in the liver” as used herein refers to a nucleotide sequence which is complementary or partially complementary to at least a portion of a microRNA expressed in the liver. Similarly, a “target sequence of a microRNA expressed in the heart” or “target sequence binding to a microRNA expressed in the heart” or “binding site of a microRNA expressed in the heart” as used herein refers to a nucleotide sequence which is complementary or partially complementary to at least a portion of a microRNA expressed in the heart.

A portion of a microRNA expressed in the liver or a portion of a microRNA expressed in the heart, as described herein, means a nucleotide sequence of at least four, at least five, at least six or at least seven consecutive nucleotides of said microRNA. The binding site sequence can have perfect complementarity to at least a portion of an expressed microRNA, meaning that the sequences are a perfect match without any mismatch occurring. Alternatively, the binding site sequence can be partially complementary to at least a portion of an expressed microRNA, meaning that one mismatch in four, five, six or seven consecutive nucleotides may occur. Partially complementary binding sites preferably contain perfect or near perfect complementarity to the seed region of the microRNA, meaning that no mismatch (perfect complementarity) or one mismatch per four, five, six or seven consecutive nucleotides (near perfect complementarity) may occur between the seed region of the microRNA and its binding site. The seed region of the microRNA consists of the 5′ region of the microRNA from about nucleotide 2 to about nucleotide 8 of the microRNA. The portion as described herein is preferably the seed region of said microRNA. Degradation of the messenger RNA (mRNA) containing the target sequence for a microRNA expressed in the liver or a microRNA expressed in the heart may be through the RNA interference pathway or via direct translational control (inhibition) of the mRNA. This invention is in no way limited by the pathway ultimately utilized by the miRNA in inhibiting expression of the transgene or encoded protein.

In the context of the invention, a target sequence that binds to microRNAs expressed in the liver may be selected from SEQ ID NO's 12 or 14-20 or may be a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 12 or 14-20.

In a preferred embodiment, the target sequence of a microRNA expressed in the liver is SEQ ID NO: 12 or a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 12. In a further embodiment, at least one copy of a target sequence of a microRNA expressed in the liver, as described in SEQ ID NO: 12 or 14-20, is present in the gene construct of the invention. In a further embodiment, two, three, four, five, six, seven or eight copies of a target sequence of a microRNA expressed in the liver, as described in SEQ ID NO: 12 or 14-20, are present in the gene construct of the invention. In a preferred embodiment, one, two, three, four, five, six, seven or eight copies of the sequence miRT-122a (SEQ ID NO: 12) are present in the gene construct of the invention. A preferred number of copies of a target sequence of a microRNA expressed in the liver is four.

A target sequence of a microRNA expressed in the liver as used herein exerts at least a detectable level of activity of a target sequence of a microRNA expressed in the liver as known to a person of skill in the art. An activity of a target sequence of a microRNA expressed in the liver is to bind to its cognate microRNA expressed in the liver and, when operatively linked to a transgene, to mediate detargeting of transgene expression in the liver. This activity may be assessed by measuring the levels of transgene expression in the liver on the level of the mRNA or the protein by standard assays known to a person of skill in the art, such as qPCR, Western blot analysis or ELISA.

In the context of the invention, a target sequence of a microRNA expressed in the heart may be selected from SEQ ID NO's: 13 or 21-25 or may be a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 13 or 21-25.

In a preferred embodiment, the target sequence of a microRNA expressed in the heart may be selected SEQ ID NO: 13 or may be a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 13. In a further embodiment, at least one copy of a target sequence of a microRNA expressed in the heart, as described in SEQ ID NO: 13 or 21-25, is present in the gene construct of the invention. In a further embodiment, two, three, four, five, six, seven or eight copies of a target sequence of a microRNA expressed in the heart, as described in SEQ ID NO: 13 or 21-25, are present in the gene construct of the invention. In a preferred embodiment, one, two, three, four, five, six, seven or eight copies of a nucleotide sequence encoding miRT-1 (SEQ ID NO: 13), are present in the gene construct of the invention. A preferred number of copies of a target sequence of a microRNA expressed in the heart is four.

A target sequence of a microRNA expressed in the heart as used herein exerts at least a detectable level of activity of a target sequence of a microRNA expressed in the heart as known to a person of skill in the art. An activity of a target sequence of a microRNA expressed in the heart is to bind to its cognate microRNA expressed in the heart and, when operatively linked to a transgene, to mediate detargeting of transgene expression in the heart. This activity may be assessed by measuring the levels of transgene expression in the heart on the level of the mRNA or the protein by standard assays known to a person of skill in the art, such as qPCR, Western blot analysis or ELISA.

In some embodiments, at least one copy of a target sequence of a microRNA expressed in the liver, as described in SEQ ID NO: 12 or 14-20, and at least one copy of a target sequence of a microRNA expressed in the heart, as described in SEQ ID NO: 13 or 21-25, are present in the gene construct of the invention. In a further embodiment, two, three, four, five, six, seven or eight copies of a target sequence of a microRNA expressed in the liver, as described in SEQ ID NO: 12 or 14-20, and two, three, four, five, six, seven or eight copies of a target sequence of a microRNA expressed in the heart, as described in SEQ ID NO: 13 or 21-25, are present in the gene construct of the invention. In a further embodiment one, two, three, four, five, six, seven or eight copies of a nucleotide sequence encoding miRT-122a (SEQ ID NO: 12) and one, two, three, four, five, six, seven or eight copies nucleotide sequence encoding miRT-1 (SEQ ID NO: 13) are combined in the gene construct of the invention. In a further embodiment, four copies of a nucleotide sequence encoding miRT-122a (SEQ ID NO: 12) and four copies of nucleotide sequence encoding miRT-1 (SEQ ID NO: 13) are combined in the gene construct of the invention.

In some embodiments there is provided a gene construct as described above, wherein the target sequence of a microRNA expressed in the liver and the target sequence of a microRNA expressed in the heart is selected from a group consisting of sequences SEQ ID NO: 12 to 25 and/or combinations thereof. In some embodiments there is provided a gene construct as described above, wherein the target sequence of a microRNA expressed in the heart is selected from SEQ ID NO's: 13 and 21-25 and a target sequence of a microRNA expressed in the liver is selected from SEQ ID NO's: 12 and 14-20. In some embodiments there is provided a gene construct as described above, wherein the gene construct comprises a target sequence of microRNA-122a and a target sequence of microRNA-1.

In some embodiments, a ubiquitous promoter as described herein is selected from the group consisting of a CAG promoter, a CMV promoter, a mini-CMV promoter, a β-actin promoter, a rous-sarcoma-virus (RSV) promoter, an elongation factor 1 alpha (EF1α) promoter, an early growth response factor-1 (Egr-1) promoter, an Eukaryotic Initiation Factor 4A (eIF4A) promoter, a ferritin heavy chain-encoding gene (FerH) promoter, a ferritin heavy light-encoding gene (FerL) promoter, a glyceraldehyde-3-phosphate dehydrogenase (GAPDH) promoter, a GRP78 promoter, a GRP94 promoter, a heat-shock protein 70 (hsp70) promoter, an ubiquitin B promoter, a SV40 promoter, a Beta-Kinesin promoter, a ROSA26 promoter and a PGK-1 promoter.

In a preferred embodiment, the ubiquitous promoter is a CAG promoter. CAG promoters are demonstrated in the examples to be suitable for use in a gene construct according to the invention.

In some embodiments, a CAG promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 27.

Another preferred ubiquitous promoter is a cytomegalovirus (CMV) promoter. CMV promoters are demonstrated in the examples to be suitable for use in a gene construct according to the invention.

In some embodiments, a CMV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 28. Preferably said CMV promoter is used together with an intronic sequence. In some embodiments, an intronic sequence comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 26.

Another preferred ubiquitous promoter is a mini-CMV promoter. In some embodiments, a mini-CMV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 36.

Another preferred ubiquitous promoter is an EF1α promoter. In some embodiments, an EF1α promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 37.

Another preferred ubiquitous promoter is an RSV promoter. In some embodiments, an RSV promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 38.

In some embodiments, the nucleotide sequence encoding FGF21 is operably linked to a tissue-specific promoter.

A description of “tissue-specific promoter” has been provided under the section entitled “general information”.

In a preferred embodiment, the tissue-specific promoter is a CNS-specific promoter, more preferably a brain-specific promoter.

In some embodiments, a CNS-specific promoter as described herein is selected from the group consisting of a Synapsin 1 promoter, a Neuron-specific enolase (NSE) promoter, a Calcium/calmodulin-dependent protein kinase II (CaMKII) promoter, a tyrosine hydroxylase (TH) promoter, a Forkhead Box A2 (FOXA2) promoter, an alpha-internexin (INA) promoter, a Nestin (NES) promoter, a Glial fibrillary acidic protein (GFAP) promoter, an Aldehyde Dehydrogenase 1 Family Member L1 (ALDH1L1) promoter, a myelin-associated oligodendrocyte basic protein (MOBP) promoter, a Homeobox Protein 9 (HB9) promoter, a Myelin basic protein (MBP) promoter and a Gonadotropin-releasing hormone (GnRH) promoter.

In some embodiments, a brain-specific promoter as described herein is selected from the group consisting of a Synapsin 1 promoter, a Neuron-specific enolase (NSE) promoter, a Calcium/calmodulin-dependent protein kinase II (CaMKII) promoter, a tyrosine hydroxylase (TH) promoter, a Forkhead Box A2 (FOXA2) promoter, an alpha-internexin (INA) promoter, a Nestin (NES) promoter, a Glial fibrillary acidic protein (GFAP) promoter, an Aldehyde Dehydrogenase 1 Family Member L1 (ALDH1L1) promoter, a myelin-associated oligodendrocyte basic protein (MOBP) promoter, a Myelin basic protein (MBP) promoter and a Gonadotropin-releasing hormone (GnRH) promoter.

In a preferred embodiment, the CNS- and/or brain-specific promoter is a synapsin 1 promoter. In some embodiments, a synapsin 1 promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 39.

Another preferred CNS- and/or brain-specific promoter is a calcium/calmodulin-dependent protein kinase II (CaMKII) promoter. In some embodiments, a calcium/calmodulin-dependent protein kinase II (CaMKII) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 40.

Another preferred CNS- and/or brain-specific promoter is a Glial fibrillary acidic protein (GFAP) promoter. In some embodiments, a Glial fibrillary acidic protein (GFAP) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 41.

Another preferred CNS- and/or brain-specific promoter is a Nestin promoter. In some embodiments, a Nestin promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 42.

Another preferred CNS-specific promoter is a Homeobox Protein 9 (HB9) promoter. In some embodiments, a Homeobox Protein 9 (HB9) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 43.

Another preferred CNS- and/or brain-specific promoter is a tyrosine hydroxylase (TH) promoter. In some embodiments, a tyrosine hydroxylase (TH) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 44.

Another preferred CNS- and/or brain-specific promoter is a Myelin basic protein (MBP) promoter.

In some embodiments, a Myelin basic protein (MBP) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 45.

In some embodiments, CNS-, and/or brain-specific promoters as described herein direct expression of said nucleotide sequence in at least one cell of the CNS and/or brain. Preferably, said promoter directs expression in at least 10%, 20%, 30%, 40%, 40%, 60%, 70%, 80%, 90%, or 100% of cells of the CNS and/or the brain. A CNS- and/or brain-specific promoter, as used herein, also encompasses promoters directing expression in a specific region or cellular subset of the CNS and/or brain. Accordingly, CNS- and/or brain specific promoters as described herein may also direct expression in at least 10%, 20%, 30%, 40%, 40%, 60%, 70%, 80%, 90%, or 100% of cells of the hippocampus, the cerebellum, the cortex, the hypothalamus and/or the olfactory bulb. Expression may be assessed as described under the section entitled “general information”.

In another embodiment, the tissue-specific promoter is a liver-specific promoter. In some embodiments, a liver-specific promoter as described herein is selected from the group consisting of an albumin promoter, a major urinary protein promoter, a phosphoenolpyruvate carboxykinase (PEPCK) promoter, a liver enriched protein activator promoter, a transthyretin promoter, a thyroxine binding globulin promoter, an apolipoprotein A1 promoter, a liver fatty acid binding protein promoter a phenylalanine hydroxylase promoter and a human α1-antitrypsin (hAAT) promoter.

In a preferred embodiment, the liver-specific promoter is a human α1-antitrypsin (hAAT) promoter. In some embodiments, a human α1-antitrypsin (hAAT) promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 64.

Preferably said hAAT promoter is used together with an intronic sequence. A preferred intronic sequence is a hepatocyte control region (HCR) enhancer from apolipoprotein E. A most preferred intronic sequence is the HCR enhancer from apolipoprotein E as defined in SEQ ID NO: 65. In this context an intronic sequence may be replaced by a nucleotide sequence comprising a nucleotide sequence that has at least 60% sequence identity or similarity with SEQ ID NO: 53. A preferred nucleotide sequence has at least 60%, 65%, 70%, 75%, 80%, 85%, 90%, 95%, 97%, 98%, 99%, 100% identity with SEQ ID NO: 65. In an embodiment, said hAAT promoter is used together with one, two, three, four or five copies of an intronic sequence. In a preferred embodiment, said hAAT promoter is used together with one, two, three, four or five copies of the HCR enhancer from apolipoprotein E as defined in SEQ ID NO: 65.

In some embodiments, liver-specific promoters as described herein direct expression of said nucleotide sequence in at least one cell of the liver. Preferably, said promoter directs expression in at least 10%, 20%, 30%, 40%, 40%, 60%, 70%, 80%, 90%, or 100% of cells of the liver. A liver-specific promoter, as used herein, also encompasses promoters directing expression in a specific region or cellular subset of the liver. Accordingly, liver-specific promoters as described herein may also direct expression in at least 10%, 20%, 30%, 40%, 40%, 60%, 70%, 80%, 90%, or 100% of cells of the hippocampus, the cerebellum, the cortex, the hypothalamus and/or the olfactory bulb. Expression may be assessed as described under the section entitled “general information”.

In another embodiment, the tissue-specific promoter is an adipose tissue-specific promoter. In some embodiments, an adipose tissue-specific promoter as described herein is selected from the group consisting an adipocyte protein 2 (aP2, also known as fatty acid binding protein 4 (FABP4)) promoter, a PPARy promoter, an adiponectin promoter, a phosphoenolpyruvate carboxykinase (PEPCK) promoter, a promoter derived from human aromatase cytochrome p450 (p450arom), a mini/aP2 promoter (composed of the adipose-specific aP2 enhancer and the basal aP2 promoter), an uncoupling protein 1 (UCP1) promoter, a mini/UCP1 promoter (composed of the adipose-specific UCP1 enhancer and the basal UCP1 promoter), an adipsin promoter, a leptin promoter, and the Foxa-2 promoter.

In a preferred embodiment, the adipose tissue-specific promoter is a mini/aP2 promoter or a mini/UCP1 promoter. In some embodiments, a mini/aP2 promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 66. In some embodiments, a mini/UCP1 promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 67.

In another embodiment, the tissue-specific promoter is a skeletal muscle promoter. In some embodiments, a skeletal muscle promoter as described herein is selected from the group consisting a myosin light-chain promoter, a myosin heavy-chain promoter, a desmin promoter, a muscle creatine kinase (MCK) promoter, a smooth muscle alpha-actin promoter, a CK6 promoter, a Unc-45 Myosin Chaperone B promoter, a basal MCK promoter in combination with copies of the MCK enhancer, and an Enh358MCK promoter (combination of the MCK enhancer with the 358 bp proximal promoter of the MCK gene).

In a preferred embodiment, the skeletal muscle promoter is a C5-12 promoter. In some embodiments, a C5-12 promoter comprises, consists essentially of, or consists of a nucleotide sequence that has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity with SEQ ID NO: 68.

A promoter as used herein (especially when the promoter sequence is described as having a minimal identity percentage with a given SEQ ID NO) should exert at least an activity of a promoter as known to a person of skill in the art. Preferably a promoter described as having a minimal identity percentage with a given SEQ ID NO should control transcription of the nucleotide sequence to which it is operably linked (i.e. at least a nucleotide sequence encoding a FGF21) as assessed in an assay known to a person of skill in the art. For example, such assay could involve measuring expression of the transgene. Expression may be assessed as described under the section entitled “general information”.

In some embodiments, a gene construct as described herein has at least one of elements a), b), c), d) and e):
(a) a liver-specific promoter;
(b) an adipose tissue-specific promoter;
(c) a combination of an ubiquitous promoter and at least one nucleotide sequence encoding a target sequence of a microRNA expressed in the liver and at least one nucleotide sequence encoding a target sequence of a microRNA expressed in the heart, optionally wherein said combination enables specific expression in adipose tissue;
(d) a skeletal muscle promoter; and
(e) a combination of an ubiquitous promoter and an adeno-associated virus (AAV) vector sequence, optionally wherein said combination enables specific expression in skeletal muscle.

Additional sequences may be present in the gene construct of the invention. Exemplary additional sequences suitable herein include inverted terminal repeats (ITRs), an SV40 polyadenylation signal (SEQ ID NO: 32), a rabbit β-globin polyadenylation signal (SEQ ID NO: 33), a CMV enhancer sequence (SEQ ID NO: 29) and a chimeric intron composed of introns from human β-globin and immunoglobulin heavy chain genes (SEQ ID NO: 26). Within the context of the invention, “ITRs” is intended to encompass one 5′ITR and one 3′ITR, each being derived from the genome of an AAV. Preferred ITRs are from AAV2 and are represented by SEQ ID NO: 30 (5′ ITR) and SEQ ID NO: 31 (3′ ITR). Within the context of the invention, it is encompassed to use the CMV enhancer sequence (SEQ ID NO: 29) and the CMV promoter sequence (SEQ ID NO: 28) as two separate sequences or as a single sequence (SEQ ID NO: 34). Each of these additional sequences may be present in a gene construct according to the invention. In some embodiments, there is provided a gene construct comprising a nucleotide sequence encoding FGF21 as described herein, further comprising one 5′ITR and one 3′ITR, preferably AAV2 ITRs, more preferably the AAV2 ITRs represented by SEQ ID NO: 30 (5′ ITR) and SEQ ID NO: 31 (3′ ITR). In some embodiments, there is provided a gene construct comprising a nucleotide sequence encoding FGF21 as described herein, further comprising a polyadenylation signal, preferably an SV40 polyadenylation signal (preferably represented by SEQ ID NO: 32) and/or a rabbit β-globin polyadenylation signal (preferably represented by SEQ ID NO: 33).

Optionally, additional nucleotide sequences may be operably linked to the nucleotide sequence(s) encoding an FGF21, such as nucleotide sequences encoding signal sequences, nuclear localization signals, expression enhancers, and the like.

In some embodiments, there is provided a gene construct comprising a nucleotide sequence encoding FGF21, optionally wherein the gene construct does not comprise a target sequence of a microRNA. In some embodiments, there is provided a gene construct comprising a nucleotide sequence encoding FGF21, optionally wherein the gene construct does not comprise a target sequence of a microRNA expressed in a tissue where the expression of FGF21 is wanted to be prevented.

In some embodiments, the level of sequence identity or similarity as used herein is preferably 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Expression Vector

Gene constructs described herein can be placed in expression vectors. Thus, in another aspect there is provided an expression vector comprising a gene construct as described in any of the preceding embodiments. In some embodiments, an expression vector as described herein is for use in therapy. In a preferred embodiment, an expression vector as described herein is for use in the treatment and/or prevention of a central nervous system (CNS) disorder or disease, or a condition associated therewith.

A description of “expression vector” has been provided under the section entitled “general information”.

In some embodiments, the expression vector is a viral expression vector. A description of “viral expression vector” has been provided under the section entitled “general information”.

A viral vector may be a viral vector selected from the group consisting of adenoviral vectors, adeno-associated viral vectors, retroviral vectors and lentiviral vectors. An adenoviral vector is also known as an adenovirus derived vector, an adeno-associated viral vector is also known as an adeno-associated virus derived vector, a retroviral vector is also known as a retrovirus derived vector and a lentiviral vector is also known as a lentivirus derived vector. A preferred viral vector is an adeno-associated viral vector. A description of “adeno-associated viral vector” has been provided under the section entitled “general information”.

In some embodiments, the vector is an adeno-associated vector or adeno-associated viral vector or an adeno-associated virus derived vector (AAV) selected from the group consisting of AAV of serotype 1 (AAV1), AAV of serotype 2 (AAV2), AAV of serotype 3 (AAV3), AAV of serotype 4 (AAV4), AAV of serotype 5 (AAV5), AAV of serotype 6 (AAV6), AAV of serotype 7 (AAV7), AAV of serotype 8 (AAV8), AAV of serotype 9 (AAV9), AAV of serotype rh10 (AAVrh10), AAV of serotype rh8 (AAVrh8), AAV of serotype Cb4 (AAVCb4), AAV of serotype rh74 (AAVrh74), AAV of serotype DJ (AAVDJ), AAV of serotype 2/5 (AAV2/5), AAV of serotype 2/1 (AAV2/1), AAV of serotype 1/2 (AAV1/2) and AAV of serotype Anc80 (AAVAnc80).

In a preferred embodiment, the vector is an AAV of serotype 1, 8 or 9 (AAV1, AAV8, or AAV9). In a more preferred embodiment the vector is an AAV of serotype 1 or 8 (AAV1 or AAV8). These AAV serotypes 1, 8 and 9 are demonstrated in the examples to be suitable for use as an expression vector according to the invention.

In a preferred embodiment the expression vector is an AAV1 and comprises a gene construct comprising a nucleotide sequence encoding FGF21 operably linked to a CMV promoter. Optionally, the gene construct further includes an SV40 polyadenylation signal (SEQ ID NO: 32). This vector is demonstrated in the examples to be suitable for use as an expression vector according to the invention, particularly by intramuscular administration.

In another preferred embodiment the expression vector is an AAV1 and comprises a gene construct comprising a nucleotide sequence encoding FGF21 operably linked to a CAG promoter. Optionally, the gene construct further includes a rabbit β-globin polyadenylation signal (SEQ ID NO: 33). This vector is demonstrated in the examples to be suitable for use as an expression vector according to the invention, particularly by intra-CSF (cerebrospinal fluid) administration.

In another preferred embodiment the expression vector is an AAV8 and comprises a gene construct comprising a nucleotide sequence encoding FGF21 operably linked to a CAG promoter and at least one target sequence of microRNA-122a (SEQ ID NO: 12) and at least one target sequence of microRNA-1 (SEQ ID NO: 13). Optionally, the gene construct further includes a rabbit β-globin polyadenylation signal (SEQ ID NO: 33). This vector is demonstrated in the examples to be suitable for use as an expression vector according to the invention, particularly by intra-adipose tissue such as intra-eWAT (epididimal white adipose tissue) administration.

In another preferred embodiment the expression vector is an AAV9 and comprises a gene construct comprising a nucleotide sequence encoding FGF21 operably linked to a CAG promoter and at least one target sequence of microRNA-122a (SEQ ID NO: 12) and at least one target sequence of microRNA-1 (SEQ ID NO: 13). This vector is demonstrated in the examples to be suitable for use as an expression vector according to the invention, particularly by intra-CSF (cerebrospinal fluid) administration.

Composition

In a further aspect there is provided a composition comprising a gene construct as described above and/or an expression vector as described above, optionally further comprising one or more pharmaceutically acceptable ingredients.

Such composition may be called a gene therapy composition. Preferably, the composition is a pharmaceutical composition.

As used herein, “pharmaceutically acceptable ingredients” include pharmaceutically acceptable carriers, fillers, preservatives, solubilizers, vehicles, diluents and/or excipients. Accordingly, the one or more pharmaceutically acceptable ingredients may be selected from the group consisting of pharmaceutically acceptable carriers, fillers, preservatives, solubilizers, vehicles, diluents and/or excipients. Such pharmaceutically acceptable carriers, fillers, preservatives, solubilizers, vehicles, diluents and/or excipients may for instance be found in Remington: The Science and Practice of Pharmacy, 22nd edition. Pharmaceutical Press (2013), incorporated herein by reference.

In some embodiments, a composition as described herein is for use in therapy. In a preferred embodiment, a composition as described herein is for use in the treatment and/or prevention of a central nervous system (CNS) disorder or disease, or a condition associated therewith.

A further compound may be present in a composition of the invention. Said compound may help in delivery of the composition. Suitable compounds in this context are: compounds capable of forming complexes, nanoparticles, micelles and/or liposomes that deliver each constituent as described herein, complexed or trapped in a vesicle or liposome through a cell membrane. Many of these compounds are known in the art. Suitable compounds comprise polyethylenimine (PEI), or similar cationic polymers, including polypropyleneimine or polyethylenimine copolymers (PECs) and derivatives; synthetic amphiphiles (SAINT-18); Lipofectin™, DOTAP. A person of skill in the art will know which type of formulation is the most appropriate for a composition as described herein.

Method and Use

Provided herein are gene constructs, expression vectors and compositions for use in the treatment and/or prevention of a central nervous system (CNS) disorder or disease, or a condition associated therewith, as described elsewhere herein.

In a further aspect there is provided a method of treatment and/or prevention of a central nervous system (CNS) disorder or disease, or a condition associated therewith, comprising administering a gene construct, an expression vector and/or a composition as described herein. In some embodiments, administering a gene construct, an expression vector or a composition means administering to a subject such as a subject in need thereof. In a preferred embodiment, a therapeutically effective amount of a gene construct, an expression vector or a composition is administered.

As used herein, an “effective amount” is an amount sufficient to exert beneficial or desired results.

In a further aspect there is provided a use of a gene construct, an expression vector or a composition as described herein, for the manufacture of a medicament for the treatment and/or prevention of a central nervous system (CNS) disorder or disease, or a condition associated therewith.

In a further aspect there is provided a use of a gene construct, an expression vector or a composition as described herein, for the treatment and/or prevention of a central nervous system (CNS) disorder or disease, or a condition associated therewith.

In a preferred embodiment, the central nervous system disorder or disease, or a condition associated therewith, is associated with and/or caused by aging and/or a metabolic disorder or disease, preferably obesity and/or diabetes.

In some embodiments according to a gene construct for use, an expression vector for use, a composition for use, a method and a use according to the invention, the central nervous system (CNS) disorder or disease, or a condition associated therewith, may be neuroinflammation, neurodegeneration, cognitive decline and/or a disease or condition associated therewith.

In some embodiments, the disease or condition associated with neuroinflammation, neurodegeneration and/or cognitive decline is selected from the group consisting of: a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases, preferably selected from the group consisting of Alzheimer's disease, Parkinson's disease, Parkinson-like disease and Huntington's disaese, more preferably selected from the group consisting of Alzheimer's disease and Parkinson's disease, most preferably Alzheimer's disease.

In some embodiments according to a gene construct for use, an expression vector for use, a composition for use, a method and a use according to the invention, the central nervous system (CNS) disorder or disease, or a condition associated therewith, may be a behavioral disorder. In a preferred embodiment the behavioral disorder is an anxiety disorder and/or a depressive disorder. Non-limiting examples of anxiety disorders encompassed by the invention are generalized anxiety disorder, specific phobia, social anxiety disorder, separation anxiety disorder, agoraphobia, panic disorder, and selective mutism. Non-limiting examples of depressive disorders encompassed by the invention are major depressive disorder (MDD), anhedonia, atypical depression, melancholic depression, psychotic major depression, catatonic depression, postpartum depression, premenstrual dysphoric disorder, seasonal affective disorder, dyshtymia, double depression, depressive disorder not otherwise specified, depressive personality disorder, recurrent brief depression and minor depressive disorder. In some embodiments, an anxiety disorder may also relate to anxiety-like behavior and a depressive disorder may also relate to depressive-like behavior.

In some embodiments according to a gene construct for use, an expression vector for use, a composition for use, a method and a use according to the invention, the central nervous system (CNS) disorder or disease, or a condition associated therewith, may be a neuromuscular disorder, preferably the neuromuscular disorder is, or is associated with, declined muscle function, declined muscle strength, declined coordination, declined balance, and/or hypoactivity.

In another aspect there is provided a method for improving memory and/or learning in a subject, the method comprising administering to the subject a gene construct as described herein and/or an expression vector as described herein and/or a composition as described herein. In a preferred embodiment, an effective amount of a gene construct, an expression vector or a composition is administered. In a preferred embodiment, the subject to be treated is an elderly subject and/or a subject diagnosed with a metabolic disorder or disease, preferably obesity and/or diabetes. In some embodiments, memory may be recognition and/or recall memory, preferably recognition memory. In some embodiments, memory may be sensory memory, short-term memory and/or long-term memory, preferably short-term memory and/or long-term memory. In some embodiments, memory may be implicit (or procedural) and/or explicit (or declarative) memory. In a preferred embodiment, memory may also by spatial memory. In some embodiments, learning may be spatial learning. Further description of the different types of memory are included in the section entitled “General information”.

In another aspect there is provided a method for improving muscle function, muscle strength, coordination, balance and/or hypoactivity in a subject, the method comprising administering to the subject a gene construct as described herein and/or an expression vector as described herein and/or a composition as described herein. In a preferred embodiment, an effective amount of a gene construct, an expression vector or a composition is administered. In a preferred embodiment, the subject to be treated is an elderly subject and/or a subject diagnosed with a metabolic disorder or disease, preferably obesity and/or diabetes.

In preferred embodiments according to a gene construct for use, an expression vector for use, a composition for use, a method and a use according to the invention, the subject to be treated is an elderly subject and/or a subject diagnosed with a metabolic disorder or disease. In other words, in some embodiments according to a gene construct for use, an expression vector for use, a composition for use, a method and a use according to the invention, the central nervous system disorder or disease, or a condition associated therewith, is associated with and/or caused by aging and/or a metabolic disorder or disease. Complications of a metabolic disorder or disease may also be encompassed.

As used herein, an elderly subject may preferably mean a subject with age 50 years or older, preferably 55 years or older, more preferably 60 years or older and most preferably 65 years or older.

In other embodiments according to a gene construct for use, an expression vector for use, a composition for use, a method and a use according to the invention, the subject to be treated is not an elderly subject and/or is a subject with age 50 years or younger, 45 years or younger, 40 years or younger, 35 years or younger, 30 years or younger, 25 years or younger.

In other embodiments according to a gene construct for use, an expression vector for use, a composition for use, a method and a use according to the invention, the subject to be treated is a subject not diagnosed with a metabolic disorder or disease. In other words, in some embodiments according to a gene construct for use, an expression vector for use, a composition for use, a method and a use according to the invention, the central nervous system disorder or disease, or a condition associated therewith, is not associated with and/or caused by aging and/or a metabolic disorder or disease.

Metabolic disorders and diseases may include metabolic syndrome, diabetes, obesity, obesity-related comorbidities, diabetes-related comorbidities, hyperglycaemia, insulin resistance, glucose intolerance, hepatic steatosis, alcoholic liver diseases (ALD), non-alcoholic fatty liver disease (NAFLD), non-alcoholic steatohepatitis (NASH), coronary heart disease (CHD), hyperlipidemia, atherosclerosis, endocrinopathies, osteosarcopenic obesity syndrome (OSO), diabetic nephropathy, chronic kidney disease (CKD), cardiac hypertrophy, diabetic retinopathy, diabetic nephropathy, diabetic neuropathy, arthritis, sepsis, ocular neovascularization, neurodegeneration, dementia, and may also include depression, adenoma, carcinoma. Diabetes may include prediabetes, hyperglycaemia, Type 1 diabetes, Type 2 diabetes, maturity-onset diabetes of the young (MODY), monogenic diabetes, neonatal diabetes, gestational diabetes, brittle diabetes, idiopathic diabetes, drug- or chemical-induced diabetes, Stiff-man syndrome, lipoatrophic diabetes, latent autoimmune diabetes in adults (LADA). Obesity may include overweight, central/upper body obesity, peripheral/lower body obesity, morbid obesity, osteosarcopenic obesity syndrome (OSO), pediatric obesity, Mendelian (monogenic) syndromic obesity, Mendelian non-syndromic obesity, polygenic obesity. Preferred metabolic disorders or diseases are obesity and/or a diabetes.

In some embodiments according to a gene construct for use, an expression vector for use, a composition for use, a method and a use according to the invention, the subject to be treated is a subject at risk of developing a central nervous system (CNS) disorder or disease, or a condition associated therewith

Within the context of gene constructs for use, expression vectors for use, compositions for use, methods and uses according to the invention, the therapy and/or treatment and/or medicament may involve expression of FGF21 in the CNS and/or transduction of the CNS, preferably the brain. In some embodiments, expression of FGF21 in the brain may mean expression of FGF21 in the hypothalamus and/or the cortex and/or the hippocampus and/or the cerebellum and/or the olfactory bulb. Accordingly, expression of FGF21 in the brain may mean expression of FGF21 in at least one or at least two or at least three or all brain regions selected from the group consisting of the hypothalamus, the cortex, the hippocampus, the cerebellum and the olfactory bulb. In some embodiments, expression in and/or transduction of the CNS and/or the brain and/or the hypothalamus and/or the cortex and/or the hippocampus and/or the cerebellum and/or the olfactory bulb may mean specific expression in and/or specific transduction of the CNS and/or the brain and/or the hypothalamus and/or the cortex and/or the hippocampus and/or the cerebellum and/or the olfactory bulb. In an embodiment, expression does not involve expression in the liver, pancreas, adipose tissue, skeletal muscle and/or heart. In some embodiments, expression does not involve expression in at least one, at least two, at least three, at least four or all organs selected from the group consisting of the liver, pancreas, adipose tissue, skeletal muscle and heart. A description of CNS- and/or brain- and/or hypothalamus and/or cortex- and/or hippocampus- and/or cerebellum- and/or olfactory bulb-specific expression has been provided under the section entitled “general information”.

Within the context of gene constructs for use, expression vectors for use, compositions for use, methods and uses according to the invention, the therapy and/or treatment and/or medicament may involve expression of FGF21 in the liver and/or transduction of the liver. In some embodiments, expression in and/or transduction of the liver may mean specific expression in and/or specific transduction of the liver. In an embodiment, expression does not involve expression in the CNS, brain, pancreas, adipose tissue, skeletal muscle and/or heart. In some embodiments, expression does not involve expression in at least one, at least two, at least three, at least four or all organs selected from the group consisting of the CNS, brain, pancreas, adipose tissue, skeletal muscle and heart. A description of liver-specific expression has been provided under the section entitled “general information”.

Within the context of gene constructs for use, expression vectors for use, compositions for use, methods and uses according to the invention, the therapy and/or treatment and/or medicament may involve expression of FGF21 in the muscle and/or transduction of the muscle. In some embodiments, expression in and/or transduction of the muscle may mean specific expression in and/or specific transduction of the muscle. In an embodiment, expression does not involve expression in the CNS, brain, liver, pancreas, adipose tissue and/or heart. In some embodiments, expression does not involve expression in at least one, at least two, at least three, at least four or all organs selected from the group consisting of the CNS, brain, liver, pancreas, adipose tissue and heart. A description of muscle-specific expression has been provided under the section entitled “general information”.

Within the context of gene constructs for use, expression vectors for use, compositions for use, methods and uses according to the invention, the therapy and/or treatment and/or medicament may involve expression of FGF21 in the adipose tissue and/or transduction of the adipose tissue. In some embodiments, expression in and/or transduction of the adipose tissue may mean specific expression in and/or specific transduction of the adipose tissue. In an embodiment, expression does not involve expression in the CNS, brain, liver, pancreas, skeletal muscle and/or heart. In some embodiments, expression does not involve expression in at least one, at least two, at least three, at least four or all organs selected from the group consisting of the CNS, brain, liver, pancreas, skeletal muscle and heart. A description of adipose tissue-specific expression has been provided under the section entitled “general information”.

In a preferred embodiment of gene constructs for use, expression vectors for use, compositions for use, methods and uses according to the invention, the therapy and/or treatment and/or medicament may involve at least one of:
expression of FGF21 in the CNS, preferably the brain;
expression of FGF21 in a peripheral body organ, preferably the muscle, adipose tissue and/or liver, more preferably the muscle and/or adipose tissue; and
increased circulating levels of FGF21.

Within the context of gene constructs for use, expression vectors for use, compositions for use, methods and uses according to the invention, “involving the expression of a gene construct” may be replaced by “causing the expression of a gene construct” or “inducing the expression of a gene construct” or “involving transduction”.

Within the context of gene constructs for use, expression vectors for use, compositions for use, methods and uses according to the invention, the therapy and/or treatment and/or medicament may involve expression of FGF21 in the muscle and/or transduction of the muscle, preferably skeletal muscle, such as the quadriceps, gastrocnemius and/or tibialis.

Within the context of gene constructs for use, expression vectors for use, compositions for use, methods and uses according to the invention, the therapy and/or treatment and/or medicament may involve expression of FGF21 in the adipose tissue and/or transduction of the adipose tissue, preferably white adipose tissue (WAT).

Within the context of gene constructs for use, expression vectors for use, compositions for use, methods and uses according to the invention, the therapy and/or treatment and/or medicament may involve increased circulating levels of FGF21. Circulating levels of FGF21 can be measured in the serum according to methods known in the art such as ELISA, for example as described in the experimental part.

In some embodiments of gene constructs for use, expression vectors for use, compositions for use, methods and uses according to the invention, the method or use does not involve expression of FGF21 in the CNS and/or does not involve transduction of the CNS.

In a preferred embodiment, a treatment or a therapy or a use or the administration of a medicament as described herein does not have to be repeated. In some embodiments, a treatment or a therapy or a use or the administration of a medicament as described herein may be repeated each year or each 2, 3, 4, 5, 6, 7, 8, 9 or 10, including intervals between any two of the listed values, years.

The subject treated may be a higher mammal, such as a cat, a rodent, (preferably mice, rats, gerbils and guinea pigs, and more preferably mice and rats), a dog, or a human being.

Within the context of gene constructs for use, expression vectors for use, compositions for use, methods and uses according to the invention, a gene construct and/or an expression vector and/or a composition and/or a medicament as described herein preferably exhibits at least one, at least two, at least three, at least four, or all of the following effects:
decreasing neuroinflammation;
increasing neurogenesis;
decreasing neurodegeneration;
alleviating a symptom (as described later herein); and
improving a parameter (as described later herein).

Decreasing neuroinflammation may mean that inflammation of nervous tissue is decreased. This could be assessed using techniques known to a person of skill in the art such as the measurement of (neuro)inflammatory markers, for example as done in the experimental part. Exemplary markers that could be used in this regard are II-1b, II-6 and NfkB. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The decrease may be seen after at least one week, one month, six months, one year or more of treatment using a gene construct and/or an expression vector and/or a composition of the invention. Preferably, the decrease is observed after a single administration. In some embodiments, the decrease is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Increasing neurogenesis may mean that neurons are produced by neural stem cells. This could be assessed using techniques known to a person of skill in the art such as the measurement of neurogenesis markers. Exemplary markers that could be used in this regard are Dcx, Ncam and Sox2. In this context, “increase” (respectively “improvement”) means at least a detectable increase (respectively a detectable improvement) using an assay known to a person of skill in the art, such as assays as carried out in the experimental part. The increase may be an increase of at least 5%, at least 10%, at least 20%, at least 30%, at least 40%, at least 50%, at least 60%, at least 70%, at least 80%, at least 90% or at least 100%. The increase may be seen after at least one week, one month, six months, one year or more of treatment using a gene construct and/or an expression vector and/or a composition of the invention. Preferably, the increase is observed after a single administration. In some embodiments, the increase is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Decreasing neurodegeneration may mean that the loss of structure or function of neurons, including death of neurons, is decreased. This could be assessed using techniques known to a person of skill in the art such as immunocytochemistry, immunohistochemistry, by medical imaging techniques such as MRI, studying the neuron morphology and synaptic degeneration (by measuring density of proteins located in synapses) or by analyzing expression levels of several senescence and neurodegeneration markers. A non-limiting example of relevant markers are markers of mitochondrial dysfunction and/or oxidative stress, such as markers associated with any of the processes and pathways of Table 1. In this context, “decrease” (respectively “improvement”) means at least a detectable decrease (respectively a detectable improvement) using an assay known to a person of skill in the art, such as assays as carried out in the experimental part. The decrease may be a decrease of at least 5%, at least 10%, at least 20%, at least 30%, at least 40%, at least 50%, at least 60%, at least 70%, at least 80%, at least 90% or at least 100%. The increase may be seen after at least one week, one month, six months, one year or more of treatment using a gene construct and/or an expression vector and/or a composition of the invention. Preferably, the increase is observed after a single administration. In some embodiments, the increase is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Alleviating a symptom may mean that the progression of a typical symptom (e.g. neuroinflammation, neurodegeneration, cognitive decline, synapse loss, tau phosphorylation, loss of coordination, loss of balance, loss of muscle strength, loss of muscle function, hypoactivity, depression, anxiety, anhedonia, . . . ) has been slowed down in an individual, in a cell, tissue or organ of said individual as assessed by a physician. A decrease of a typical symptom may mean a slowdown in progression of symptom development or a complete disappearance of symptoms. Symptoms, and thus also a decrease in symptoms, can be assessed using a variety of methods, to a large extent the same methods as used in diagnosis of central nervous system disorders or diseases, or conditions associated therewith, including clinical examination and routine laboratory tests. Clinical examination may include behavioral tests and cognitive tests. Laboratory tests may include both macroscopic and microscopic methods, molecular methods, radiographic methods such as X-rays, biochemical methods, immunohistochemical methods and others. The alleviation of a symptom may be seen after at least one week, one month, six months, one year or more of treatment using a gene construct and/or an expression vector and/or a composition of the invention. Preferably, the alleviation is observed after a single administration. In some embodiments, the alleviation is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

Improving a parameter may mean improving results after behavioral test, improving the expression of serum and CSF markers, improving the expression of apoptosis/neurogenesis cell markers, etc. The improvement of a parameter may be seen after at least one week, one month, six months, one year or more of treatment using a gene construct and/or an expression vector and/or a composition of the invention. Preferably, the improvement is observed after a single administration. In some embodiments, the improvement is observed for a duration of at least one week, one month, six months, 1 year, 2 years, 3 years, 4 years, 5 years, 6 years, 7 years, 8 years, 9 years, 10 years, 12 years, 15 years, 20 years or more, preferably after a single administration.

A gene construct and/or an expression vector and/or a composition as described herein is preferably able to alleviate a symptom or a characteristic of a patient or of a cell, tissue or organ of said patient if after at least one week, one month, six months, one year or more of treatment using a gene construct and/or an expression vector and/or a composition of the invention, said symptom or characteristic has decreased (e.g. is no longer detectable or has slowed down), as described herein.

A gene construct and/or an expression vector and/or a composition as described herein may be suitable for administration to a cell, tissue and/or an organ in vivo of individuals affected by or at risk of developing a central nervous system (CNS) disorder or disease, or a condition associated therewith, and may be administered in vivo, ex vivo or in vitro. Said gene construct and/or expression vector and/or composition may be directly or indirectly administered to a cell, tissue and/or an organ in vivo of an individual affected by or at risk of developing a central nervous system (CNS) disorder or disease, or a condition associated therewith, and may be administered directly or indirectly in vivo, ex vivo or in vitro.

Within the context of gene constructs for use, expression vectors for use, compositions for use, methods and uses according to the invention, a gene construct and/or an expression vector and/or a composition may be administered by different administration modes. An administration mode may be intravenous, intramuscular, intraperitoneal, via inhalation, intranasal, intraparenchymal, intra-CSF (cerebrospinal fluid), intra-ocular, subcutaneous, intraarticular, intra-adipose tissue, oral, intrahepatic, intrasplanchnic, intra-ear, topical administration and/or via retrograde intraductal pancreatic administration. Intra-CSF administration may be performed via cisterna magna, intrathecal or intraventricular delivery. “Intra-CSF administration”, “intranasal administration”, “intraparenchymal administration”, “intra-cisterna magna administration”, “intrathecal administration” and “intraventricular administration”, as used herein, are described in the part of this application entitled “general information”.

Preferred administration modes are intramuscular, intra-adipose tissue such as intra-eWAT (epididymal white adipose tissue) and intra-CSF (cerebrospinal fluid) (via cisterna magna, intrathecal or intraventricular delivery) administration. For intra-CSF administration, injection via the cisterna magna is most preferred.

In some embodiments within the context of gene constructs for use, expression vectors for use, compositions for use, methods and uses according to the invention, a gene construct and/or an expression vector and/or a composition is not administered via intra-CSF administration.

A viral expression construct and/or a viral vector and/or a nucleic acid molecule and/or a composition of the invention may be directly or indirectly administered using suitable means known in the art. Improvements in means for providing an individual or a cell, tissue, organ of said individual with a viral expression construct and/or a viral vector and/or a nucleic acid molecule and/or a composition of the invention are anticipated, considering the progress that has already thus far been achieved. Such future improvements may of course be incorporated to achieve the mentioned effect of the invention. A viral expression construct and/or a viral vector and/or a nucleic acid molecule and/or a composition can be delivered as is to an individual, a cell, tissue or organ of said individual. Depending on the disease or condition, a cell, tissue or organ of said individual may be as earlier described herein. When administering a viral expression construct and/or a viral vector and/or a nucleic acid molecule and/or a composition of the invention, it is preferred that such viral expression construct and/or vector and/or nucleic acid and/or composition is dissolved in a solution that is compatible with the delivery method.

As encompassed herein, a therapeutically effective dose of a viral expression construct, vector, nucleic acid molecule and/or composition as mentioned above is preferably administered in a single and unique dose hence avoiding repeated periodical administration.

General Information

Unless stated otherwise, all technical and scientific terms used herein have the same meaning as customarily and ordinarily understood by a person of ordinary skill in the art to which this invention belongs, and read in view of this disclosure.

As used herein, the term “promoter” or “regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” and/or “repressible” promoter is a promoter that is physiologically or developmentally regulated to be induced and/or repressed, e.g. by the application of a chemical inducer or repressing signal.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence such as a promoter is operably linked to a coding sequence if it affects the transcription of the coding sequence. Operably linked means that the DNA sequences being linked are typically contiguous and, where necessary to join two protein encoding regions, contiguous and in reading frame.

As used herein, a “regulator” or “transcriptional regulator” is a protein that controls the rate of transcription of genetic information from DNA to messenger RNA, by binding to a specific DNA sequence.

The terms “protein” or “polypeptide” are used interchangeably and refer to molecules consisting of a chain of amino acids, without reference to a specific mode of action, size, 3-dimensional structure or origin.

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site.

“Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

In amino acid sequences as described herein, amino acids or “residues” are denoted by three-letter symbols. These three-letter symbols as well as the corresponding one-letter symbols are well known to the person skilled in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid, as described herein.

Sequence Identity

In the context of the invention, a nucleic acid molecule such as a nucleic acid molecule encoding an FGF21 is represented by a nucleic acid or nucleotide sequence which encodes a protein fragment or a polypeptide or a peptide or a derived peptide. In the context of the invention, an FGF21 protein fragment or a polypeptide or a peptide or a derived peptide as Fibroblast growth factor 21 (FGF21) is represented by an amino acid sequence.

It is to be understood that each nucleic acid molecule or protein fragment or polypeptide or peptide or derived peptide or construct as identified herein by a given sequence identity number (SEQ ID NO) is not limited to this specific sequence as disclosed. Each coding sequence as identified herein encodes a given protein fragment or polypeptide or peptide or derived peptide or construct or is itself a protein fragment or polypeptide or construct or peptide or derived peptide.

Throughout this application, each time one refers to a specific nucleotide sequence SEQ ID NO (take SEQ ID NO: X as example) encoding a given protein fragment or polypeptide or peptide or derived peptide, one may replace it by:
i. a nucleotide sequence comprising a nucleotide sequence that has at least 60% sequence identity with SEQ ID NO: X;
ii. a nucleotide sequence the sequence of which differs from the sequence of a nucleic acid molecule of (i) due to the degeneracy of the genetic code; or
iii. a nucleotide sequence that encodes an amino acid sequence that has at least 60% amino acid identity or similarity with an amino acid sequence encoded by a nucleotide sequence SEQ ID NO: X.

Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Throughout this application, each time one refers to a specific amino acid sequence SEQ ID NO (take SEQ ID NO: Y as example), one may replace it by: a polypeptide represented by an amino acid sequence comprising a sequence that has at least 60% sequence identity or similarity with amino acid sequence SEQ ID NO: Y. Another preferred level of sequence identity or similarity is 70%. Another preferred level of sequence identity or similarity is 80%. Another preferred level of sequence identity or similarity is 90%. Another preferred level of sequence identity or similarity is 95%. Another preferred level of sequence identity or similarity is 99%.

Each nucleotide sequence or amino acid sequence described herein by virtue of its identity or similarity percentage with a given nucleotide sequence or amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively.

Each non-coding nucleotide sequence (i.e. of a promoter or of another regulatory region) could be replaced by a nucleotide sequence comprising a nucleotide sequence that has at least 60% sequence identity or similarity with a specific nucleotide sequence SEQ ID NO (take SEQ ID NO: A as example). A preferred nucleotide sequence has at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identity with SEQ ID NO: A. In a preferred embodiment, such non-coding nucleotide sequence such as a promoter exhibits or exerts at least an activity of such a non-coding nucleotide sequence such as an activity of a promoter as known to a person of skill in the art.

The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred embodiment, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Bioinformatics and the Cell: Modern Computational Approaches in Genomics, Proteomics and transcriptomics, Xia X., Springer International Publishing, New York, 2018; and Bioinformatics: Sequence and Genome Analysis, Mount D., Cold Spring Harbor Laboratory Press, New York, 2004, each incorporated herein by reference.

“Sequence identity” and “sequence similarity” can be determined by alignment of two peptide or two nucleotide sequences using global or local alignment algorithms, depending on the length of the two sequences. Sequences of similar lengths are preferably aligned using a global alignment algorithms (e.g. Needleman-Wunsch) which aligns the sequences optimally over the entire length, while sequences of substantially different lengths are preferably aligned using a local alignment algorithm (e.g. Smith-Waterman). Sequences may then be referred to as “substantially identical” or “essentially similar” when they (when optimally aligned by for example the program EMBOSS needle or EMBOSS water using default parameters) share at least a certain minimal percentage of sequence identity (as described below).

A global alignment is suitably used to determine sequence identity when the two sequences have similar lengths. When sequences have a substantially different overall length, local alignments, such as those using the Smith-Waterman algorithm, are preferred. EMBOSS needle uses the Needleman-Wunsch global alignment algorithm to align two sequences over their entire length (full length), maximizing the number of matches and minimizing the number of gaps. EMBOSS water uses the Smith-Waterman local alignment algorithm. Generally, the EMBOSS needle and EMBOSS water default parameters are used, with a gap open penalty=10 (nucleotide sequences)/10 (proteins) and gap extension penalty=0.5 (nucleotide sequences)/0.5 (proteins). For nucleotide sequences the default scoring matrix used is DNAfull and for proteins the default scoring matrix is Blosum62 (Henikoff & Henikoff, 1992, PNAS 89, 915-919, incorporated herein by reference). Alternatively percentage similarity or identity may be determined by searching against public databases, using algorithms such as FASTA, BLAST, etc. Thus, the nucleic acid and protein sequences of some embodiments of the present invention can further be used as a “query sequence” to perform a search against public databases to, for example, identify other family members or related sequences. Such searches can be performed using the BLASTn and BLASTx programs (version 2.0) of Altschul, et al. (1990) J. Mol. Biol. 215:403-10, incorporated herein by reference. BLAST nucleotide searches can be performed with the NBLAST program, score=100, wordlength=12 to obtain nucleotide sequences homologous to oxidoreductase nucleic acid molecules of the invention. BLAST protein searches can be performed with the BLASTx program, score=50, wordlength=3 to obtain amino acid sequences homologous to protein molecules of the invention. To obtain gapped alignments for comparison purposes, Gapped BLAST can be utilized as described in Altschul et al., (1997) Nucleic Acids Res. 25(17): 3389-3402, incorporated herein by reference. When utilizing BLAST and Gapped BLAST programs, the default parameters of the respective programs (e.g., BLASTx and BLASTn) can be used. See the homepage of the National Center for Biotechnology Information accessible on the world wide web at www.ncbi.nlm.nih.gov/. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

Gene or Coding Sequence

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′-nontranslated sequence (3′-end) e.g. comprising a polyadenylation- and/or transcription termination site. A chimeric or recombinant gene (such as a FGF21 gene) is a gene not normally found in nature, such as a gene in which for example the promoter is not associated in nature with part or all of the transcribed DNA region. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

A “transgene” is herein described as a gene or a coding sequence or a nucleic acid molecule (i.e. a molecule encoding a FGF21) that has been newly introduced into a cell, i.e. a gene that may be present but may normally not be expressed or expressed at an insufficient level in a cell. In this context, “insufficient” means that although said FGF21 is expressed in a cell, a condition and/or disease as described herein could still be developed. In this case, the invention allows the over-expression of a FGF21. The transgene may comprise sequences that are native to the cell, sequences that naturally do not occur in the cell and it may comprise combinations of both. A transgene may contain sequences coding for a FGF21 and/or additional proteins as earlier identified herein that may be operably linked to appropriate regulatory sequences for expression of the sequences coding for a FGF21 in the cell. Preferably, the transgene is not integrated into the host cell's genome.

Promoter

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

A “ubiquitous promoter” is active in substantially all tissues, organs and cells of an organism. A “organ-specific” or “tissue-specific” promoter is a promoter that is active in a specific type of organ or tissue, respectively. Organ-specific and tissue-specific promoters regulate expression of one or more genes (or coding sequence) primarily in one organ or tissue, but can allow detectable level (“leaky”) expression in other organs or tissues as well. Leaky expression in other organs or tissues means at least one-fold, at least two-fold, at least three-fold, at least four-fold or at least five-fold lower, but still detectable expression as compared to the organ-specific or tissue-specific expression, as evaluated on the level of the mRNA or the protein by standard assays known to a person of skill in the art (e.g. qPCR, Western blot analysis, ELISA). The maximum number of organs or tissues where leaky expression may be detected is five, six, seven or eight.

A “CNS- or brain-specific promoter” is a promoter that is capable of initiating transcription in the CNS and/or brain, whilst still allowing for any leaky expression in other (maximum five, six, seven or eight) organs and parts of the body. Transcription in the CNS and/or brain can be detected in relevant areas, such as the hypothalamus, cortex, hippocampus, cerebellum and olfactory bulb, and cells, such as neurons and/or glial cells.

In the context of the invention, CNS- and/or brain- and/or hypothalamus and/or cortex- and/or hippocampus- and/or cerebellum- and/or olfactory bulb-specific promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the CNS and/or the brain and/or the hypothalamus and/or the cortex and/or the hippocampus and/or the cerebellum and/or the olfactory bulb as compared to other organs or tissues. Other organs or tissues may be the liver, pancreas, adipose tissue, skeletal muscle, heart, kidney, colon, hematopoietic tissue, lung, ovary, spleen, stomach, testis and others. Preferably, other organs are the liver and the heart. Expression may be assessed as described elsewhere under the section entitled “general information”.

Throughout the application, where CNS- and/or brain- and/or hypothalamus and/or cortex- and/or hippocampus- and/or cerebellum- and/or olfactory bulb-specific is mentioned in the context of expression, cell-type specific expression of the cell type(s) making up the CNS and/or the brain and/or the hypothalamus and/or the cortex and/or the hippocampus and/or the cerebellum and/or the olfactory bulb is also envisaged, respectively.

A “liver-specific promoter” is a promoter that is capable of initiating transcription in the liver, whilst still allowing for any leaky expression in other (maximum five, six, seven or eight) organs and parts of the body. Transcription in the liver can be detected in liver tissue and liver cells, such as hepatocytes, Kupffer cells and/or oval cells.

In the context of the invention, liver-specific promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the liver as compared to other organs or tissues. Other organs or tissues may be the CNS, brain, pancreas, adipose tissue, skeletal muscle, heart, kidney, colon, hematopoietic tissue, lung, ovary, spleen, stomach, testis and others. Preferably, other organs are the heart. Expression may be assessed as described elsewhere under the section entitled “general information”.

Throughout the application, where liver-specific is mentioned in the context of expression, cell-type specific expression of the cell type(s) making up the liver (including hepatocytes, Kupffer cells and/or oval cells) is also envisaged, respectively.

An “adipose tissue-specific promoter” is a promoter that is capable of initiating transcription in the adipose tissue, whilst still allowing for any leaky expression in other (maximum five, six, seven or eight) organs and parts of the body. Transcription in the adipose tissue can be detected in adipose tissue adipose tissue cells, such as white adipocytes, brown adipocytes, beige adipocytes, preadipocytes, stromal vascular cells.

In the context of the invention, adipose tissue-specific promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the adipose tissue as compared to other organs or tissues. Other organs or tissues may be the CNS, brain, pancreas, liver, skeletal muscle, heart, kidney, colon, hematopoietic tissue, lung, ovary, spleen, stomach, testis and others. Preferably, other organs are the heart. Expression may be assessed as described elsewhere under the section entitled “general information”.

Throughout the application, where adipose tissue-specific is mentioned in the context of expression, cell-type specific expression of the cell type(s) making up the adipose tissue (including white adipocytes, brown adipocytes, beige adipocytes, preadipocytes, stromal vascular cells) is also envisaged, respectively.

A “skeletal muscle promoter” is a promoter that is capable of initiating transcription in the skeletal muscle, whilst still allowing for any leaky expression in other (maximum five, six, seven or eight) organs and parts of the body. Transcription in the skeletal muscle can be detected in skeletal muscle tissue and skeletal muscle cells, such as myocytes, myoblasts, satellite cells.

In the context of the invention, skeletal muscle promoters may be promoters that are capable of driving the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the skeletal muscle as compared to other organs or tissues. Other organs or tissues may be the CNS, brain, pancreas, adipose tissue, liver, heart, kidney, colon, hematopoietic tissue, lung, ovary, spleen, stomach, testis and others. Preferably, other organs are the heart. Expression may be assessed as described elsewhere under the section entitled “general information”.

Throughout the application, where skeletal muscle is mentioned in the context of expression, cell-type specific expression of the cell type(s) making up the skeletal muscle (including myocytes, myoblasts, satellite cells) is also envisaged, respectively.

Operably Linked

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame. Linking can be accomplished by ligation at convenient restriction sites or at adapters or linkers inserted in lieu thereof, or by gene synthesis.

microRNA

As used herein, “microRNA” or “miRNA” or “miR” has its customary and ordinary meaning as understood by one of skill in the art in view of this disclosure. A microRNA is a small non-coding RNA molecule found in plants, animals and some viruses, that may function in RNA silencing and post-transcriptional regulation of gene expression. A target sequence of a microRNA may be denoted as “miRT”. For example, a target sequence of microRNA-1 or miRNA-1 or miR-1 may be denoted as miRT-1.

Proteins and Amino Acids

The terms “protein” or “polypeptide” or “amino acid sequence” are used interchangeably and refer to molecules consisting of a chain of amino acids, without reference to a specific mode of action, size, 3-dimensional structure or origin. In amino acid sequences as described herein, amino acids or “residues” are denoted by three-letter symbols. These three-letter symbols as well as the corresponding one-letter symbols are well known to a person of skill in the art and have the following meaning: A (Ala) is alanine, C (Cys) is cysteine, D (Asp) is aspartic acid, E (Glu) is glutamic acid, F (Phe) is phenylalanine, G (Gly) is glycine, H (His) is histidine, I (Ile) is isoleucine, K (Lys) is lysine, L (Leu) is leucine, M (Met) is methionine, N (Asn) is asparagine, P (Pro) is proline, Q (Gin) is glutamine, R (Arg) is arginine, S (Ser) is serine, T (Thr) is threonine, V (Val) is valine, W (Trp) is tryptophan, Y (Tyr) is tyrosine. A residue may be any proteinogenic amino acid, but also any non-proteinogenic amino acid such as D-amino acids and modified amino acids formed by post-translational modifications, and also any non-natural amino acid.

CNS and Brain

As used herein, “central nervous system” or “CNS” refers to the part of the nervous system that comprises the brain and the spinal cord, to which sensory impulses are transmitted and from which motor impulses pass out, and which coordinates the activity of the entire nervous system.

As used herein, “brain” refers to the central organ of the nervous system and consists of the cerebrum, the brainstem and the cerebellum. It controls most of the activities of the body, processing, integrating, and coordinating the information it receives from the sense organs, and making decisions as to the instructions sent to the rest of the body.

In particular, as used herein, ‘hypothalamus” refers to a region of the forebrain below the thalamus which coordinates both the autonomic nervous system and the activity of the pituitary, controlling body temperature, thirst, hunger, and other homeostatic systems, and involved in sleep and emotional activity. “Hippocampus”, as used herein, belongs to the limbic system and plays important roles in the consolidation of information from short-term memory to long-term memory, and in spatial memory that enables navigation. The hippocampus is located under the cerebral cortex (allocortical) and in primates in the medial temporal lobe. The “cortex” or “cerebral cortex”, as used herein, is the outer layer of neural tissue of the cerebrum of the brain, in humans and other mammals. It plays a key role in memory, attention, perception, awareness, thought, language, and consciousness. “Cerebellum”, as used herein, refers to a major feature in the hindbrain of all vertebrates. In humans, it plays an important role in motor control. It may also be involved in some cognitive functions such as attention and language as well as in regulating fear and pleasure responses. “Olfactory bulb”, as used herein, refers to an essential structure in the olfactory system (the system devoted to the sense of smell. The olfactory bulb sends information to be further processed in the amygdala, the orbitofrontal cortex (OFC) and the hippocampus where it plays a role in emotion, memory and learning.

Gene Constructs

Gene constructs as described herein could be prepared using any cloning and/or recombinant DNA techniques, as known to a person of skill in the art, in which a nucleotide sequence encoding said FGF21 is expressed in a suitable cell, e.g. cultured cells or cells of a multicellular organism, such as described in Ausubel et al., “Current Protocols in Molecular Biology”, Greene Publishing and Wiley-Interscience, New York (1987) and in Sambrook and Russell (2001, supra); both of which are incorporated herein by reference in their entirety. Also see, Kunkel (1985) Proc. Natl. Acad. Sci. 82:488 (describing site directed mutagenesis) and Roberts et al. (1987) Nature 328:731-734 or Wells, J. A., et al. (1985) Gene 34: 315 (describing cassette mutagenesis).

Expression Vectors

The phrase “expression vector” or “vector” generally refers to a tool in molecular biology used to obtain gene expression in a cell, for example by introducing a nucleotide sequence that is capable of effecting expression of a gene or a coding sequence in a host compatible with such sequences. An expression vector carries a genome that is able to stabilize and remain episomal in a cell. Within the context of the invention, a cell may mean to encompass a cell used to make the construct or a cell wherein the construct will be administered. Alternatively, a vector is capable of integrating into a cell's genome, for example through homologous recombination or otherwise.

These expression vectors typically include at least suitable promoter sequences and optionally, transcription termination signals. An additional factor necessary or helpful in effecting expression can also be used as described herein. A nucleic acid or DNA or nucleotide sequence encoding a FGF21 is incorporated into a DNA construct capable of introduction into and expression in an in vitro cell culture. Specifically, a DNA construct is suitable for replication in a prokaryotic host, such as bacteria, e.g., E. coli, or can be introduced into a cultured mammalian, plant, insect, (e.g., Sf9), yeast, fungi or other eukaryotic cell lines.

A DNA construct prepared for introduction into a particular host may include a replication system recognized by the host, an intended DNA segment encoding a desired polypeptide, and transcriptional and translational initiation and termination regulatory sequences operably linked to the polypeptide-encoding segment. The term “operably linked” has already been described herein. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of a polypeptide. Generally, a DNA sequence that is operably linked are contiguous, and, in the case of a signal sequence, both contiguous and in reading frame. However, enhancers need not be contiguous with a coding sequence whose transcription they control. Linking is accomplished by ligation at convenient restriction sites or at adapters or linkers inserted in lieu thereof, or by gene synthesis. The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment. In most cases, the replication system is only functional in the cell that is used to make the vector (bacterial cell as E. Coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in, yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media. Alternatively, a host cell is a cell that is part of a multicellular organism such as a transgenic plant or animal.

The selection of an appropriate promoter sequence generally depends upon the host cell selected for the expression of a DNA segment. Examples of suitable promoter sequences include prokaryotic, and eukaryotic promoters well known in the art (see, e.g. Sambrook and Russell, 2001, supra). A transcriptional regulatory sequence typically includes a heterologous enhancer or promoter that is recognised by the host. The selection of an appropriate promoter depends upon the host, but promoters such as the trp, lac and phage promoters, tRNA promoters and glycolytic enzyme promoters are known and available (see, e.g. Sambrook and Russell, 2001, supra). An expression vector includes the replication system and transcriptional and translational regulatory sequences together with the insertion site for the polypeptide encoding segment. In most cases, the replication system is only functional in the cell that is used to make the vector (bacterial cell as E. coli). Most plasmids and vectors do not replicate in the cells infected with the vector. Examples of workable combinations of cell lines and expression vectors are described in Sambrook and Russell (2001, supra) and in Metzger et al. (1988) Nature 334: 31-36. For example, suitable expression vectors can be expressed in yeast, e.g. S. cerevisiae, e.g., insect cells, e.g., Sf9 cells, mammalian cells, e.g., CHO cells and bacterial cells, e.g., E. coli. A cell may thus be a prokaryotic or eukaryotic host cell. A cell may be a cell that is suitable for culture in liquid or on solid media. Alternatively, a host cell is a cell that is part of a multicellular organism such as a transgenic plant or animal.

Viral Vector

A viral vector or a viral expression vector a viral gene therapy vector is a vector that comprises a gene construct as described herein.

A viral vector or a viral gene therapy vector is a vector that is suitable for gene therapy. Vectors that are suitable for gene therapy are described in Anderson 1998, Nature 392: 25-30; Walther and Stein, 2000, Drugs 60: 249-71; Kay et al., 2001, Nat. Med. 7: 33-40; Russell, 2000, J. Gen. Virol. 81: 2573-604; Amado and Chen, 1999, Science 285: 674-6; Federico, 1999, Curr. Opin. Biotechnol. 10: 448-53; Vigna and Naldini, 2000, J. Gene Med. 2: 308-16; Marin et al., 1997, Mol. Med. Today 3: 396-403; Peng and Russell, 1999, Curr. Opin. Biotechnol. 10: 454-7; Sommerfelt, 1999, J. Gen. Virol. 80: 3049-64; Reiser, 2000, Gene Ther. 7: 910-3; and references cited therein. A particularly suitable gene therapy vector includes an adenoviral and adeno-associated virus (AAV) vector. These vectors infect a wide number of dividing and non-dividing cell types including synovial cells and liver cells. The episomal nature of the adenoviral and AAV vectors after cell entry makes these vectors suited for therapeutic applications, (Russell, 2000, J. Gen. Virol. 81: 2573-2604; Goncalves, 2005, Virol J. 2(1):43) as indicated above. AAV vectors are even more preferred since they are known to result in very stable long-term expression of transgene expression (up to 9 years in dog (Niemeyer et al, Blood. 2009 Jan. 22; 113(4):797-806) and ˜10 years in human (Buchlis, G. et al., Blood. 2012 Mar. 29; 119(13):3038-41). Preferred adenoviral vectors are modified to reduce the host response as reviewed by Russell (2000, supra). Method for gene therapy using AAV vectors are described by Wang et al., 2005, J Gene Med. March 9 (Epub ahead of print), Mandel et al., 2004, Curr Opin Mol Ther. 6(5):482-90, and Martin et al., 2004, Eye 18(11):1049-55, Nathwani et al, N Engl J Med. 2011 Dec. 22; 365(25):2357-65, Apparailly et al, Hum Gene Ther. 2005 April; 16(4):426-34.

Another suitable gene therapy vector includes a retroviral vector. A preferred retroviral vector for application in the present invention is a lentiviral based expression construct. Lentiviral vectors have the ability to infect and to stably integrate into the genome of dividing and non-dividing cells (Amado and Chen, 1999 Science 285: 674-6). Methods for the construction and use of lentiviral based expression constructs are described in U.S. Pat. Nos. 6,165,782, 6,207,455, 6,218,181, 6,277,633 and 6,323,031 and in Federico (1999, Curr Opin Biotechnol 10: 448-53) and Vigna et al. (2000, J Gene Med 2000; 2: 308-16).

Other suitable gene therapy vectors include an adenovirus vector, a herpes virus vector, a polyoma virus vector or a vaccinia virus vector.

Adeno-Associated Virus Vector (AAV Vector)

The terms “adeno associated virus”, “AAV virus”, “AAV virion”, “AAV viral particle” and “AAV particle”, used as synonyms herein, refer to a viral particle composed of at least one capsid protein of AAV (preferably composed of all capsid protein of a particular AAV serotype) and an encapsulated polynucleotide of the AAV genome. If the particle comprises a heterologous polynucleotide (i.e. a polynucleotide different from a wild-type AAV genome, such as a transgene to be delivered to a mammalian cell) flanked by AAV inverted terminal repeats, then they are typically known as a “AAV vector particle” or “AAV viral vector” or “AAV vector”. AAV refers to a virus that belongs to the genus Dependovirus family Parvoviridae. The AAV genome is approximately 4.7 Kb in length and it consists of single strand deoxyribonucleic acid (ssDNA) that can be positive or negative detected. The invention also encompasses the use of double stranded AAV also called dsAAV or scAAV. The genome includes inverted terminal repeats (ITR) at both ends of the DNA strand, and two open reading frames (ORFs): rep and cap. The frame rep is made of four overlapping genes that encode proteins Rep necessary for AAV lifecycle. The frame cap contains nucleotide sequences overlapping with capsid proteins: VP1, VP2 and VP3, which interact to form a capsid of icosahedral symmetry (see Carter and Samulski, 2000, and Gao et al, 2004). A preferred viral vector or a preferred gene therapy vector is an AAV vector. An AAV vector as used herein preferably comprises a recombinant AAV vector (rAAV vector). A “rAAV vector” as used herein refers to a recombinant vector comprising part of an AAV genome encapsidated in a protein shell of capsid protein derived from an AAV serotype as explained herein. Part of an AAV genome may contain the inverted terminal repeats (ITR) derived from an adeno-associated virus serotype, such as AAV1, AAV2, AAV3, AAV4, AAV5 and others. Preferred ITRs are those of AAV2 which are represented by sequences comprising, consisting essentially of, or consisting of SEQ ID NO: 30 (5′ ITR) and SEQ ID NO: 31 (3′ ITR). The invention also preferably encompasses the use of a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 30 as 5′ ITR and a sequence having at least 80% (or at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%) identity with SEQ ID NO: 31 as 3′ ITR.

Protein shell comprised of capsid protein may be derived from any AAV serotype. A protein shell may also be named a capsid protein shell. rAAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, at least 85%, at least 90%, at least 95%, at least 97%, at least 98%, at least 99% or 100% sequence identity with wild type sequences or may be altered by for example by insertion, mutation, deletion or substitution of nucleotides, as long as they remain functional. In this context, functionality refers to the ability to direct packaging of the genome into the capsid shell and then allow for expression in the host cell to be infected or target cell. In the context of the present invention a capsid protein shell may be of a different serotype than the rAAV vector genome ITR.

A nucleic acid molecule represented by a nucleic acid sequence of choice is preferably inserted between the rAAV genome or ITR sequences as identified above, for example an expression construct comprising an expression regulatory element operably linked to a coding sequence and a 3′ termination sequence. Said nucleic acid molecule may also be called a transgene.

“AAV helper functions” generally refers to the corresponding AAV functions required for rAAV replication and packaging supplied to the rAAV vector in trans. AAV helper functions complement the AAV functions which are missing in the rAAV vector, but they lack AAV ITRs (which are provided by the rAAV vector genome). AAV helper functions include the two major ORFs of AAV, namely the rep coding region and the cap coding region or functional substantially identical sequences thereof. Rep and Cap regions are well known in the art, see e.g. Chiorini et al. (1999, J. of Virology, Vol 73(2): 1309-1319) or U.S. Pat. No. 5,139,941, incorporated herein by reference. The AAV helper functions can be supplied on an AAV helper construct. Introduction of the helper construct into the host cell can occur e.g. by transformation, transfection, or transduction prior to or concurrently with the introduction of the rAAV genome present in the rAAV vector as identified herein. The AAV helper constructs of the invention may thus be chosen such that they produce the desired combination of serotypes for the rAAV vector's capsid protein shell on the one hand and for the rAAV genome present in said rAAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via plasmids, as described in U.S. Pat. No. 6,531,456 incorporated herein by reference.

“Transduction” refers to the delivery of a FGF21 into a recipient host cell by a viral vector. For example, transduction of a target cell by a rAAV vector of the invention leads to transfer of the rAAV genome contained in that vector into the transduced cell. “Host cell” or “target cell” refers to the cell into which the DNA delivery takes place, such as the muscle cells of a subject. AAV vectors are able to transduce both dividing and non-dividing cells.

Production of an AA V Vector

The production of recombinant AAV (rAAV) for vectorizing transgenes have been described previously. See Ayuso E, et al., Curr. Gene Ther. 2010; 10:423-436, Okada T, et al., Hum. Gene Ther. 2009; 20:1013-1021, Zhang H, et al., Hum. Gene Ther. 2009; 20:922-929, and Virag T, et al., Hum. Gene Ther. 2009; 20:807-817. These protocols can be used or adapted to generate the AAV of the invention. In one embodiment, the producer cell line is transfected transiently with the polynucleotide of the invention (comprising the expression cassette flanked by ITRs) and with construct(s) that encodes rep and cap proteins and provides helper functions. In another embodiment, the cell line supplies stably the helper functions and is transfected transiently with the polynucleotide of the invention (comprising the expression cassette flanked by ITRs) and with construct(s) that encodes rep and cap proteins. In another embodiment, the cell line supplies stably the rep and cap proteins and the helper functions and is transiently transfected with the polynucleotide of the invention. In another embodiment, the cell line supplies stably the rep and cap proteins and is transfected transiently with the polynucleotide of the invention and a polynucleotide encoding the helper functions. In yet another embodiment, the cell line supplies stably the polynucleotide of the invention, the rep and cap proteins and the helper functions. Methods of making and using these and other AAV production systems have been described in the art. See Muzyczka N, et al., U.S. Pat. No. 5,139,941, Zhou X, et al., U.S. Pat. No. 5,741,683, Samulski R, et al., U.S. Pat. No. 6,057,152, Samulski R, et al., U.S. Pat. No. 6,204,059, Samulski R, et al., U.S. Pat. No. 6,268,213, Rabinowitz J, et al., U.S. Pat. No. 6,491,907, Zolotukhin S, et al., U.S. Pat. No. 6,660,514, Shenk T, et al., U.S. Pat. No. 6,951,753, Snyder R, et al., U.S. Pat. No. 7,094,604, Rabinowitz J, et al., U.S. Pat. No. 7,172,893, Monahan P, et al., U.S. Pat. No. 7,201,898, Samulski R, et al., U.S. Pat. No. 7,229,823, and Ferrari F, et al., U.S. Pat. No. 7,439,065.

The rAAV genome present in a rAAV vector comprises at least the nucleotide sequences of the inverted terminal repeat regions (ITRs) of one of the AAV serotypes (preferably the ones of serotype AAV2 as disclosed earlier herein), or nucleotide sequences substantially identical thereto or nucleotide sequences having at least 60% identity thereto, and nucleotide sequence encoding a FGF21 (under control of a suitable regulatory element) inserted between the two ITRs. A vector genome requires the use of flanking 5′ and a 3′ ITR sequences to allow for efficient packaging of the vector genome into the rAAV capsid.

The complete genome of several AAV serotypes and corresponding ITR has been sequenced (Chiorini et al. 1999, J. of Virology Vol. 73, No. 2, p 1309-1319). They can be either cloned or made by chemical synthesis as known in the art, using for example an oligonucleotide synthesizer as supplied e.g. by Applied Biosystems Inc. (Fosters, CA, USA) or by standard molecular biology techniques. The ITRs can be cloned from the AAV viral genome or excised from a vector comprising the AAV ITRs. The ITR nucleotide sequences can be either ligated at either end to the nucleotide sequence encoding one or more therapeutic proteins using standard molecular biology techniques, or the AAV sequence between the ITRs can be replaced with the desired nucleotide sequence. Preferably, the rAAV genome as present in a rAAV vector does not comprise any nucleotide sequences encoding viral proteins, such as the rep (replication) or cap (capsid) genes of AAV. This rAAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

The rAAV genome as present in said rAAV vector further comprises a promoter sequence operably linked to the nucleotide sequence encoding a FGF21.

A suitable 3′ untranslated sequence may also be operably linked to the nucleotide sequence encoding a FGF21. Suitable 3′ untranslated regions may be those naturally associated with the nucleotide sequence or may be derived from different genes, such as for example the SV40 polyadenylation signal (SEQ ID NO: 32) and the rabbit β-globin polyadenylation signal (SEQ ID NO: 33).

Expression

Expression may be assessed by any method known to a person of skill in the art. For example, expression may be assessed by measuring the levels of transgene expression in the transduced tissue on the level of the mRNA or the protein by standard assays known to a person of skill in the art, such as qPCR, RNA sequencing, Northern blot analysis, Western blot analysis, mass spectrometry analysis of protein-derived peptides or ELISA.

Expression may be assessed at any time after administration of the gene construct, expression vector or composition as described herein. In some embodiments herein, expression may be assessed after 1 week, 2 weeks, 3 weeks, 4, weeks, 5 weeks, 6 weeks, 7 weeks, 8 weeks, 9, weeks, 10 weeks, 11 weeks, 12 weeks, 14 weeks, 16 weeks, 18 weeks, 20 weeks, 22 weeks, 24 weeks, 28 weeks, 32 weeks, 36 weeks, 40 weeks, or more.

In the context of the invention, CNS- and/or brain- and/or hypothalamus and/or cortex- and/or hippocampus- and/or cerebellum- and/or olfactory bulb-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the CNS and/or the brain and/or the hypothalamus and/or the cortex and/or the hippocampus and/or the cerebellum and/or the olfactory bulb as compared to other organs or tissues. Other organs or tissues may be the liver, pancreas, adipose tissue, skeletal muscle, heart, kidney, colon, hematopoietic tissue, lung, ovary, spleen, stomach, testis and others. Preferably, other organs are the liver and/or the heart. In an embodiment, expression is not detectable in the liver, pancreas, adipose tissue, skeletal muscle and/or heart. In some embodiments, expression is not detectable in at least one, at least two, at least three, at least four or all organs selected from the group consisting of the liver, pancreas, adipose tissue, skeletal muscle, heart, kidney, colon, hematopoietic tissue, lung, ovary, spleen, stomach and testis. Expression may be assessed as described above.

Throughout the application, where CNS- and/or brain- and/or hypothalamus and/or cortex- and/or hippocampus- and/or cerebellum- and/or olfactory bulb-specific is mentioned in the context of expression, cell-type specific expression of the cell type(s) making up the CNS and/or the brain and/or the hypothalamus and/or the cortex and/or the hippocampus and/or the cerebellum and/or the olfactory bulb is also envisaged, respectively.

In the context of the invention, liver-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the liver as compared to other organs or tissues. Other organs or tissues may be the CNS, brain, pancreas, adipose tissue, skeletal muscle, heart, kidney, colon, hematopoietic tissue, lung, ovary, spleen, stomach, testis and others. Preferably, other organs are the heart. In an embodiment, expression is not detectable in the CNS, brain, pancreas, adipose tissue, skeletal muscle and/or heart. In some embodiments, expression is not detectable in at least one, at least two, at least three, at least four or all organs selected from the group consisting of the CNS, brain, pancreas, adipose tissue, skeletal muscle, heart, kidney, colon, hematopoietic tissue, lung, ovary, spleen, stomach and testis. Throughout the application, where liver-specific is mentioned in the context of expression, cell-type specific expression of the cell type(s) making up the liver is also envisaged, respectively.

In the context of the invention, muscle-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the muscle as compared to other organs or tissues. Other organs or tissues may be the CNS, brain, liver, pancreas, adipose tissue, heart, kidney, colon, hematopoietic tissue, lung, ovary, spleen, stomach, testis and others. Preferably, other organs are the liver and/or the heart. In an embodiment, expression is not detectable in the CNS, brain, liver, pancreas, adipose tissue, and/or heart. In some embodiments, expression is not detectable in at least one, at least two, at least three, at least four or all organs selected from the group consisting of the CNS, brain, liver, pancreas, adipose tissue, heart, kidney, colon, hematopoietic tissue, lung, ovary, spleen, stomach and testis.

Throughout the application, where muscle-specific is mentioned in the context of expression, cell-type specific expression of the cell type(s) making up the muscle is also envisaged, respectively. In the context of the invention, adipose tissue-specific expression refers to the preferential or predominant (at least 10% higher, at least 20% higher, at least 30% higher, at least 40% higher, at least 50% higher, at least 60% higher, at least 70% higher, at least 80% higher, at least 90% higher, at least 100% higher, at least 150% higher, at least 200% higher or more) expression of FGF21 in the adipose tissue as compared to other organs or tissues. Other organs or tissues may be the CNS, brain, liver, pancreas, skeletal muscle, heart, kidney, colon, hematopoietic tissue, lung, ovary, spleen, stomach, testis and others. Preferably, other organs are the liver and/or the heart. In an embodiment, expression is not detectable in the CNS, brain, liver, pancreas, skeletal muscle and/or heart. In some embodiments, expression is not detectable in at least one, at least two, at least three, at least four or all organs selected from the group consisting of the CNS, brain, liver, pancreas, skeletal muscle, heart, kidney, colon, hematopoietic tissue, lung, ovary, spleen, stomach and testis. Expression may be assessed as described above.

Throughout the application, where adipose tissue-specific is mentioned in the context of expression, cell-type specific expression of the cell type(s) making up the adipose tissue is also envisaged, respectively.

Administration

As used herein, “intra-CSF administration” means direct administration into the CSF, located in the subarachnoid space between the arachnoid and pia mater layers of the meninges surrounding the brain. Intra-CSF administration can be performed via intra-cisterna magna, intraventricular or intrathecal administration. As used herein, “intra-cisterna magna administration” means administration into the cisterna magna, an opening of the subarachnoid space located between the cerebellum and the dorsal surface of the medulla oblongata. As used herein, “intraventricular administration” means administration into the either of both lateral ventricles of the brain As used herein, “intrathecal administration” involves the direct administration into the CSF within the intrathecal space of the spinal column. As used herein, “intraparenchymal administration” means local administration directly into any region of the brain parenchyma. As used herein, “intranasal administration” means administration by way of the nasal structures.

Intramuscular administration means administration directly in the muscle, preferably the skeletal muscle. Intra-adipose tissue administration means administration directly in the adipose tissue.

Codon Optimization

“Codon optimization”, as used herein, refers to the processes employed to modify an existing coding sequence, or to design a coding sequence, for example, to improve translation in an expression host cell or organism of a transcript RNA molecule transcribed from the coding sequence, or to improve transcription of a coding sequence. Codon optimization includes, but is not limited to, processes including selecting codons for the coding sequence to suit the codon preference of the expression host organism. For example, to suit the codon preference of mammalians, preferably of murine, canine or human expression hosts. Codon optimization also eliminates elements that potentially impact negatively RNA stability and/or translation (e.g. termination sequences, TATA boxes, splice sites, ribosomal entry sites, repetitive and/or GC rich sequences and RNA secondary structures or instability motifs). In some embodiments, codon-optimized sequences show at least 3%, 5%, 10%, 15%, 20%, 25%, 30%, 40%, 50%, 60%, 70%, 80%, 90%, 100% or more increase in gene expression, transcription, RNA stability and/or translation compared to the original, not codon-optimized sequence.

Memory

Memory is generally understood to be the faculty of the brain by which data or information is encoded, stored, and retrieved when needed. Different types or memory have been described. One possible distinction involves sensory memory, short-term memory and long-term memory. Sensory memory holds sensory information less than one second after an item is perceived. Short-term (also known as working memory) memory allows recall for a period of several seconds to a minute, typically without rehearsal. Long-term memory, on the contrary, can store much larger quantities of information for a potentially unlimited duration (up to a whole life span).

Another distinction involves procedural memory (or implicit memory) and explicit memory (or declarative memory). Implicit memory is not based on the conscious recall of information, but on implicit learning, i.e. remembering how to do something. Explicit (or declarative) memory is the conscious, intentional recollection of factual information, previous experiences, and concepts.

A distinction can also be made between recall memory and recognition memory. Recognition refers to our ability to “recognize” an event or piece of information as being familiar, while recall designates the retrieval of related details from memory.

Spatial memory is a form of memory responsible for the recording of information about one's environment and spatial orientation.

In this document and in its claims, the verb “to comprise” and its conjugations is used in its non-limiting sense to mean that items following the word are included, but items not specifically mentioned are not excluded. In addition, the verb “to consist” may be replaced by “to consist essentially of” meaning that a composition as described herein may comprise additional component(s) than the ones specifically identified, said additional component(s) not altering the unique characteristic of the invention. In addition, the verb “to consist” may be replaced by “to consist essentially of” meaning that a method as described herein may comprise additional step(s) than the ones specifically identified, said additional step(s) not altering the unique characteristic of the invention.

Reference to an element by the indefinite article “a” or “an” does not exclude the possibility that more than one of the element is present, unless the context clearly requires that there be one and only one of the elements. The indefinite article “a” or “an” thus usually means “at least one”.

As used herein, with “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The word “about” or “approximately” when used in association with a numerical value (e.g. about 10) preferably means that the value may be the given value (of 10) more or less 0.1% of the value. As used herein, the term “and/or” indicates that one or more of the stated cases may occur, alone or in combination with at least one of the stated cases, up to with all of the stated cases.

Various embodiments are described herein. Each embodiment as identified herein may be combined together unless otherwise indicated.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

One skilled in the art will recognize many methods and materials similar or equivalent to those described herein, which could be used in the practice of the present invention. Indeed, the present invention is in no way limited to the methods and materials described.

The present invention is further described by the following examples which should not be construed as limiting the scope of the invention.

### DESCRIPTION OF THE FIGURES

FIG. 1. Increased FGF21 circulating levels after im administration of AAV1-CMV-moFGF21 vectors in old mice. (A) Expression levels of FGF21. The expression levels of the murine codon-optimized FGF21 coding sequence (moFGF21) were measured by RTqPCR at sacrifice in tibialis, gastrocnemius and quadriceps muscles and liver and normalized with Rplp0 values. (B) Circulating levels of FGF21 6 months post-AAV administration. Results are expressed as the mean±SEM. n=5 animals/group. ND, not detected. AU, arbitrary units. ***p<0.001 vs control (21 months old) AAV1-null treated mice.

FIG. 2. Improved neuromuscular performance after im administration of AAV1-CMV-moFGF21 vectors in old mice. (A) Rotarod test. Histogram depicts the time that mice stayed on the accelerating rotarod. Old mice treated with AAV1-CMV-moFGF21 vectors showed improved coordination and balance. (B) Hang wire test. Old mice treated with AAV1-CMV-moFGF21 vectors showed improved coordination and muscular function. (C) Maximum velocity was measured in the open field test. (D) Grip strength. Results are expressed as the mean±SEM. n=12-15 animals/group. N, newtons. g, grams of body weight. *p<0.05 and ***p<0.001 vs control (3-5 months old) untreated mice; ##p<0.01 and ###p<0.001 vs control (8-10 months old) untreated mice; $ p<0.05 and $$$ p<0.001 vs control (22-24 months old) AAV1-null treated mice.

FIG. 3. Improved cognitive function in old mice treated im with AAV1-CMV-moFGF21 vectors. The Novel Object Recognition test was performed to assess memory. The histogram depicts the discrimination index. Results are expressed as the mean±SEM. n=11-13 animals/group. *p<0.05 vs control (2 months old) untreated mice; $ p<0.05 vs control (27 months old) AAV1-null treated mice.

FIG. 4. Long-term reversion of obesity after treatment with AAV vectors encoding FGF21. (A-B) Body weight evolution in animals treated intra-eWAT with AAV8-CAG-moFGF21-dmiRT vectors (A) or im with AAV1-CMV-moFGF21 vectors (B). Results are expressed as the mean±SEM. n=6-10 animals/group. HFD, high fat diet.

FIG. 5. Increased FGF21 circulating levels after treatment with AAV vectors encoding FGF21. (A-B) Circulating levels of FGF21 six months post-AAV administration in animals treated intra-eWAT with AAV8-CAG-moFGF21-dmiRT vectors (A) or im with AAV1-CMV-moFGF21 vectors (B). (C-D) Expression levels of FGF21 in adipose tissue, skeletal muscle and the liver in the same cohorts as in (A-B). The expression levels of the murine codon-optimized FGF21 coding sequence (moFGF21) were measured at sacrifice by RTqPCR and normalized with Rplp0 values. Results are expressed as the mean±SEM. n=6-10 animals/group. ND, not detected. HFD, high fat diet. AU, arbitrary units. eWAT, epididymal white adipose tissue. iWAT, inguinal white adipose tissue. iBAT interscapular brown adipose tissue. **p<0.01 and ***p<0.001 vs control chow-fed mice; #p<0.05, ##p<0.01 and ###p<0.001 vs control HFD-fed mice; $$ p<0.01 and $$$ p<0.001 vs HFD-fed mice treated intra-eWAT with 5×10¹⁰ vg of AAV8-CAG-moFGF21-dmiRT or im with 7×10¹⁰ vg of AAV1-CMV-moFGF21; &&& p<0.001 vs HFD-fed mice treated im with 1×10¹¹ vg of AAV1-CMV-moFGF21.

FIG. 6. Increased locomotor activity in HFD-fed male mice treated intra-eWAT with AAV8-CAG-moFGF21-dmiRT vectors. Locomotor activity was assessed through the Open field test. (A) Distance travelled. (B) Maximum velocity. (C) Moving time. (D) Resting time. (E) Lines crossed. (F) Fast time. (G) Slow time. Results are expressed as the mean±SEM. n=5-15 animals/group. HFD, high fat diet. **p<0.01 and ***p<0.001 vs control (2 months old) chow-fed mice; #p<0.05 vs control (11 months old) chow-fed mice; $ p<0.05, $$ p<0.01 and $$$ p<0.001 vs control (11 months old) HFD-fed mice.

FIG. 7. Increased locomotor activity in HFD-fed male mice treated im with AAV1-CMV-moFGF21 vectors. Locomotor activity was assessed through the Open field test. (A) Distance travelled. (B) Maximum velocity. (C) Moving time. (D) Resting time. (E) Lines crossed. (F) Fast time. (G) Slow time. Results are expressed as the mean±SEM. n=5-15 animals/group. HFD, high fat diet. **p<0.01 and ***p<0.001 vs control (2 months old) chow-fed mice; #p<0.05 and ####p<0.001 vs control (11 months old) chow-fed mice; $ p<0.05, $$ p<0.01 and $$$ p<0.001 vs control (11 months old) HFD-fed mice. & p<0.05 and && p<0.01 vs HFD-fed mice treated im with 7×10¹⁰ vg of AAV1-CMV-moFGF21.

FIG. 8. Decreased anxiety in HFD-fed male mice treated with AAV vectors encoding FGF21. Anxiety was assessed through the Open field test. (A-B) The histograms depict the time that animals treated intra-eWAT with AAV8-CAG-moFGF21-dmiRT vectors (A) or im with AAV1-CMV-moFGF21 vectors (B) spent in center. Results are expressed as the mean±SEM. n=5-15 animals/group. HFD, high fat diet. *p<0.05 vs control (2 months old) chow-fed mice.

FIG. 9. Long-term reversion of obesity by im administration of AAV1-CMV-moFGF21 vectors in HFD-fed female mice. (A) Body weight evolution. (B) Circulating levels of FGF21 3 months post-AAV administration. (C) Expression levels of FGF21. The expression levels of the murine codon-optimized FGF21 coding sequence (moFGF21) were measured by RTqPCR in the tibialis skeletal muscle and in the liver at sacrifice and normalized with Rplp0 values. Results are expressed as the mean±SEM. n=9-10 animals/group. ND, not detected. HFD, high fat diet. AU, arbitrary units. *** p<0.001 vs control chow-fed mice; ###p<0.001 vs control HFD-fed mice; $$$ p<0.001 vs HFD-fed mice treated im with 1×10¹¹ vg of AAV1-CMV-moFGF21.

FIG. 10. Increased locomotor activity in HFD-fed female mice treated im with AAV1-CMV-moFGF21 vectors. Locomotor activity was assessed through the Open field test. (A) Distance travelled. (B) Maximum velocity. (C) Moving time. (D) Resting time. (E) Lines crossed. (F) Fast time. (G) Slow time. Results are expressed as the mean±SEM. n=6-9 animals/group. HFD, high fat diet. *p<0.05 and **p<0.01 vs control chow-fed mice.

FIG. 11. Decreased anxiety in HFD-fed female mice treated im with AAV1-CMV-moFGF21 vectors. Anxiety was assessed through the Open field test. The histogram depicts the time animals spent in center. Results are expressed as the mean±SEM. n=6-9 animals/group. HFD, high fat diet.

FIG. 12. Improved neuromuscular performance in HFD-fed female mice treated im with AAV1-CMV-moFGF21 vectors. (A) Rotarod test. Histogram depicts the time mice stayed on the accelerating rotarod. Mice treated with AAV1-CMV-moFGF21 vectors showed improved coordination and balance. (B) Grip strength. Results are expressed as the mean±SEM. n=6-9 animals/group. N, newtons. g, grams of body weight. HFD, high fat diet. **p<0.01 and ***p<0.001 vs control chow-fed mice; ##p<0.01 vs control HFD-fed mice; $ p<0.05 vs HFD-fed mice treated im with 1×10¹¹ vg of AAV1-CMV-moFGF21.

FIG. 13. Improved cognitive function in HFD-fed female mice treated im with AAV1-CMV-moFGF21 vectors. Memory was assessed by the Novel Object Recognition test (A) and the Y-maze test (B). Results are expressed as the mean±SEM. n=6-9 animals/group. HFD, high fat diet. ##p<0.01 vs control HFD-fed mice.

FIG. 14. Increased locomotor activity in db/db mice after AAV1-CAG-moFGF21 intra-CSF administration. (A) Distance travelled, (B) Maximum velocity and (C) Fast time was measured in the Open Field test in non-treated db/+(lean), non-treated db/db and AAV1-CAG-moFGF21-treated db/db mice at 9 weeks of age. Results are expressed as the mean±SEM, n=6 animals/group. *p<0.05, **p<0.01, ***p<0.001 vs db/+ mice and ##p<0.01 ###p<0.001 vs db/db non-treated mice.

FIG. 15. Amelioration of anxiety-like behaviour in db/db mice treated intra-CSF with AAV1-CAG-moFGF21. (A) Distance in the border and (B) distance in the center was measured in the Open Field test in non-treated db/+(lean), non-treated db/db and AAV1-CAG-moFGF21-treated db/db mice at 9 weeks of age. Results are expressed as the mean±SEM, n=6 animals/group. **p<0.01 and ***p<0.001 vs db/+ mice.

FIG. 16. Increased exploratory capacity of AAV1-CAG-moFGF21 intra-CSF-treated db/db mice. (A) Number of entries and (B) First time latency was measured in the Y-maze test in non-treated db/+(lean), non-treated db/db and AAV1-CAG-moFGF21-treated db/db mice at 10 weeks of age. Results are expressed as the mean±SEM, n=6 animals/group. *p<0.05 vs db/+ mice.

FIG. 17. Amelioration of short-term memory in db/db mice after intra-CSF gene therapy with AAV1-CAG-moFGF21 vectors. The discrimination index was measured during a novel object recognition test in non-treated db/+(lean), non-treated db/db and AAV1-CAG-moFGF21-treated db/db mice at 11 weeks of age, and calculated as explained in the General Procedures of the Examples. Results are expressed as the mean±SEM, n=6 animals/group. *p<0.05 vs db/+ mice.

FIG. 18. Expression of FGF21 in the brain of AAV1-FGF21-treated db/db mice. The expression levels of the murine codon-optimized FGF21 (moFgf21) coding sequence were measured by RTqPCR in Hypothalamus, Cortex, Hippocampus, Cerebellum and Olfactory Bulb of db/db mice, and normalized with Rplp0 values. Analyses were performed 16 weeks after intra-CSF administration of 5×10¹⁰ vg/mouse of AAV1-CAG-moFGF21 vectors. Results are expressed as the mean±SEM, n=7 animals/group. ND, non-detected.

FIG. 19. Reduction of brain inflammation in db/db mice treated with AAV9-FGF21 vectors. Expression levels of astrocyte markers (Gfap and S100b), microglia markers (Aif1) and inflammatory molecules (Nfkb, II1b and II6) were measured by RTqPCR in Hypothalamus of db/db mice, and normalized with Rplp0 values. Analyses were performed 12 weeks after intra-CSF administration of 5×10¹⁰ vg/mouse of AAV9-CAG-moFGF21-dmiRT vectors. Results are expressed as the mean±SEM, n=9 animals/group. *p<0.05 vs non-treated mice. Gfap, glial fibrillary acidic protein; S100b, calcium-binding protein B; Aif1, allograft inflammatory factor 1; Nfkb, nuclear factor kappa B; II1b, interleukin 1 beta; II6, Interleukin 6.

FIG. 20. Reduction of brain inflammation in SAMP8 mice treated with AAV9-FGF21. Expression levels of astrocyte markers (Gfap and S100b), microglia marker (Aif1) and inflammatory molecules (Nfkb, II1b and II6) were measured by RTqPCR in Hypothalamus of SAMP8 mice, and normalized with Rplp0 values. Analyses were performed 14 weeks after intra-CSF administration of 5×10¹⁰ vg/mouse of AAV9-CAG-moFGF21-dmiRT vectors. Results are expressed as the mean±SEM, n=9 animals/group. **p<0.01 vs non-treated mice. Gfap, glial fibrillary acidic protein; S100b, calcium-binding protein B; Aif1, allograft inflammatory factor 1; Nfkb, nuclear factor kappa B; II1b, interleukin 1 beta; II6, Interleukin 6.

FIG. 21. Improvement of neuromuscular performance and cognition in SAMP8 mice treated im with AAV1-CMV-moFGF21 vectors. (A) Expression levels of moFGF21 in tibialis, gastrocnemius and quadriceps muscles and liver in SAMP8 mice treated im with AAV1-CMV-moFGF21 vectors and untreated SAMP8 and SAMR1 mice. The expression levels of moFGF21 were measured at sacrifice by RTqPCR and normalized with Rplp0 values. (B) Circulating levels of FGF21 in the same cohorts as in (A). (C-D) The rotarod test was performed 24 weeks post-AAV administration. Histogram in (C) depicts the time that mice stayed on the accelerating rotarod. (D) Motor learning ability. (E-F) The Novel Object Recognition test was performed to assess short- (E) and long-term (F) memory in 7-month-old mice. Histograms depict the discrimination indexes. Results are expressed as the mean±SEM. n=7-12 animals/group. ND, not detected. AU, arbitrary units. **p<0.01 and ***p<0.001 vs SAMR1; ###p<0.001 vs non-treated SAMP8.

FIG. 22. Reduction of brain inflammation in SAMP8 mice treated im with AAV1-CMV-moFGF21. Expression levels of the inflammatory molecules Ccl19 (A) and II6a (B) were measured by RTqPCR in cortex and hippocampus of SAMP8 mice treated im with AAV1-CMV-moFGF21 vectors and of untreated SAMP8 and SAMR1 mice, and normalized with Rplp0 values. Analyses were performed at sacrifice, by 42 weeks of age. Results are expressed as the mean±SEM, n=4-5 animals/group. Ccl19, chemokine (C—C motif) ligand 19; II6, Interleukin 6. *p<0.05 vs non-treated SAMP8 mice.

FIG. 23. Improvement of memory in 3×Tg-AD mice treated im with AAV1-CMV-moFGF21 vectors. (A) Circulating levels of FGF21 in 3×Tg-AD mice treated im with AAV1-CMV-moFGF21 vectors and untreated 3×Tg-AD and B6129SF2/J mice. (B) Expression levels of moFGF21 in tibialis, gastrocnemius and quadriceps muscles and liver of the same cohorts as in (A). The expression levels of moFGF21 were measured at sacrifice by RTqPCR and normalized with Rplp0 values. (C-D) The Novel Object Recognition test was performed to assess short- (C) and long-term (D) memory in 8-month-old mice. Histograms depict the discrimination indexes. (E) insoluble amyloid β₄₀ (Aβ₄₀) levels in cortex. Results are expressed as the mean±SEM. n=3-11 animals/group. ND, not detected. AU, arbitrary units. ***p<0.001 vs B6129SF2/J; ###p<0.001 vs non-treated 3×Tg-AD.

FIG. 24. Improved neuromuscular performance and cognition in old mice treated im with different doses of AAV1-CMV-moFGF21 vectors. (A) Circulating levels of FGF21 in old mice treated im with 1×10¹¹ or 3×10¹¹ vg of AAV1-CMV-moFGF21 vectors. Analysis was performed 2 months post-AAV. (B-C) The rotarod test was performed 2 months post-AAV administration. Histogram in (B) depicts the mean time that mice stayed on the accelerating rotarod. (C) Motor learning ability. (D-E) The Novel Object Recognition test was performed to assess short- (D) and long-term (E) memory in 26-month-old mice. Histograms depict the discrimination indexes. Results are expressed as the mean±SEM. n=6-12 animals/group. ND, not detected. AU, arbitrary units. *p<0.05 and ***p<0.001 vs control.

FIG. 25. Expression of OXPHOS markers in brain of old animals treated im with AAV1-CMV-moFGF21. The expression levels of several OXPHOS markers were measured by RTqPCR in Cortex and Hippocampus of 25-month-old mice treated im with AAV1-CMV-moFGF21 vectors, and normalized with Rplp0 values. Results are expressed as the mean±SEM. n=4-6 animals/group. AU, arbitrary units; Ppargc1a, peroxisome proliferator-activated receptor gamma coactivator 1 alpha; Ppargc1b, peroxisome proliferator-activated receptor gamma coactivator 1 beta; Atp5f1a, ATP Synthase F1 Subunit alpha; mt-co1, cytochrome c oxidase 1; Cox6, cytochrome c oxidase subunit 6; Cox5a, cytochrome c oxidase subunit 5a. *p<0.05 vs control (25 months old) untreated mice.

FIG. 26. Expression of antioxidant markers in brain of old animals treated im with AAV1-CMV-moFGF21. The expression levels of different antioxidant markers were measured by RTqPCR in Cortex and Hippocampus of 25-month-old mice treated im with AAV1-CMV-moFGF21 vectors, and normalized with Rplp0 values. Results are expressed as the mean±SEM. n=4-6 animals/group. AU, arbitrary units; Nrf2, NF-E2-related factor 2; Sod1, superoxide dismutase 1; Cat, catalase. *p<0.05 vs control (25 months old) untreated mice.

FIG. 27. Treatment with AAV1-CMV-moFGF21 counteracted age-related impairment of glycolysis in brain. The expression levels of several glycolysis-related genes were measured by RTqPCR in Cortex and Hippocampus of 25-month-old mice treated im with AAV1-CMV-moFGF21 vectors, and normalized with Rplp0 values. Results are expressed as the mean±SEM. n=4-6 animals/group. AU, arbitrary units; GAPDH, glycerldehyde-3-phosphate dehydrogenase; Hk1, hexokinase 1; Pfkp, platelet isoform of phosphofructokinase; Gpd1, glycerol-3-Phosphate Dehydrogenase 1; Gpd2, glycerol-3-Phosphate Dehydrogenase 2; Pkm, pyruvate kinase M. *p<0.05, **p<0.01 and ***p<0.001 vs control (25 months old) untreated mice.

FIG. 28. Treatment with AAV1-CMV-moFGF21 increased expression of key synaptic proteins. The expression levels of key synaptic proteins were measured by RTqPCR in Cortex and Hippocampus of 25-month-old mice treated im with AAV1-CMV-moFGF21 vectors, and normalized with Rplp0 values. Results are expressed as the mean±SEM. n=4-6 animals/group. AU, arbitrary units; Syp, synaptophysin; Gria1 and Gria2, GluR1 and GluR2 subunits of the alpha-amino-3-hydroxy-5-methyl-4-isoxazole proprionic acid (AMPA)-type ionotropic glutamate receptor; Grin1, Grin2a and Grin2b, NR1, N2A and N2B subunits of the N-methyl-d-aspartate (NMDA)-type ionotropic glutamate receptor; Atf4, activating transcription factor 4. *p<0.05 and ***p<0.001 vs control (25 months old) untreated mice.

FIG. 29. Treatment with AAV1-CMV-moFGF21 increases expression of autophagy and anti-ER stress markers. The expression levels of the autophagy markers p62 (encoded by Sqstm1) and Atg5, and of the chaperone BiP were measured by RTqPCR in Cortex of 25-month-old mice treated im with AAV1-CMV-moFGF21 vectors, and normalized with Rplp0 values. Results are expressed as the mean±SEM. n=4-6 animals/group. AU, arbitrary units; Atg5, autophagy related 5. *p<0.05 vs control (25 months old) untreated mice.

FIG. 30. Treatment with AAV1-FGF21 ameliorates cholesterol homeostasis in the brain. The expression levels of cholesterol 24-hydroxylase (encoded by Cyp46a1) were measured by RTqPCR in cortex of 25-month-old mice treated im with AAV1-CMV-moFGF21 vectors, and normalized with Rplp0 values. Results are expressed as the mean±SEM. n=4-6 animals/group. AU, arbitrary units. *p<0.05 vs control (25 months old) untreated mice.

FIG. 31. Long-term reversion of obesity after intra-CSF treatment with AAV vectors encoding FGF21. (A) Body weight evolution in animals treated intra-CSF with different doses of AAV1-CAG-moFGF21-dmiRT vectors. Results are expressed as the mean±SEM. n=10 animals/group. HFD, high fat diet. (B) The expression levels of the murine codon-optimized FGF21 (moFgf21) coding sequence were measured by RTqPCR in Hypothalamus, Cortex and Hippocampus of chow and HFD-fed mice, and normalized with Rplp0 values. Analyses were performed 11 months after intra-CSF administration of 5×10⁹ and 1×10¹⁰ vg/mouse of AAV1-CAG-moFGF21 vectors. Results are expressed as the mean±SEM, n=8 animals/group. ND, non-detected.

FIG. 32. Increased locomotor activity in HFD-fed male mice treated intra-CSF with AAV1-CAG-moFGF21 vectors. Locomotor activity was assessed through the Open field test. (A) Distance travelled. (B) Maximum velocity. (C) Moving time. (D) Resting time. (E) Fast time. (F) Slow time. (G) Lines crossed. (H) Entries in center. (I) Entries in border. Results are expressed as the mean±SEM. n=at least 10 animals/group. HFD, high fat diet. *p<0.05 and **p<0.01 and vs control chow-fed mice; #p<0.05, ##p<0.01 and ###p<0.001 vs control HFD-fed mice.

FIG. 33. Decreased anxiety in HFD-fed mice treated with AAV vectors encoding FGF21. Anxiety was assessed through the Open field test and through the Elevated Plus Maze test. (A) Time in Center, (B) Time in Border, (C) Latency to Center, (D) Distance in Center and (E) Distance in Border were measured in the Open Field test in all groups of mice. (F) The histograms show the percentage of time that animals spent in the open arms or in the closed arms of the elevated plus maze. Results are expressed as the mean±SEM. n=at least 10 animals/group. HFD, high fat diet. *p<0.05 and **p<0.01 vs control chow-fed mice; #p<0.05, ##p<0.01 and ###p<0.001 vs control HFD-fed mice.

FIG. 34. Improved cognitive function in HFD-fed mice treated intra-CSF with AAV1-CAG-moFGF21 vectors. The Novel Object Recognition test was performed to assess both short and long-term memory. The histogram depicts the discrimination index in the (A) short-term trial and (B) the long-term trial. Results are expressed as the mean±SEM. n=at least 10 animals/group. HFD, high fat diet. *p<0.05 vs control chow-fed mice.

FIG. 35. Improved learning in AAV1-CAG-moFGF21 HFD-fed mice. The Barnes maze test was performed to study the learning capacity and memory of mice. (A) The graph shows the time to enter the hole in the barnes maze during the different trials. (B) The learning slope was calculated from the trial-dependent improvement to enter the hole. Results are expressed as the mean±SEM. n=at least 10 animals/group.

FIG. 36. Improved neuromuscular performance and learning in old mice treated intra-CSF with AAV1-CAG-moFGF21 vectors. (A) Histogram depicts the mean time that mice stayed on the accelerating rotarod. (B) The graph shows the trial-dependent enhancement in the time to fall the rotarod and (C) the histogram shows the slope of this trial-dependent improvement. Results are expressed as the mean±SEM. n=at least 7 animals/group. *p<0.05, **p<0.01 and ***p<0.001 vs control non-treated mice.

FIG. 37. Improved cognitive function in old mice treated intra-CSF with AAV1-CAG-moFGF21 vectors. The Novel Object Recognition test was performed to assess both short and long-term memory. The histogram depicts the discrimination index in the (A) short-term trial and (B) the long-term trial. Results are expressed as the mean±SEM. n=6 animals/group. ***p<0.001 vs control non-treated mice.

### EXAMPLES

In example 1, intramuscular administration of AAV1-CMV-moFGF21 mediates robust overexpression and increases circulating levels of FGF21 and has the following benefits:
improved coordination, balance, neuromuscular performance, strength and locomotor activity
enhanced memory and learning
decreased neurodegeneration by improving mitochondrial function and diminution of oxidative stress

In Example 2, intramuscular administration of AAV1-CMV-moFGF21 and intra-eWAT administration of AAV8-CAG-moFGF21-dmiRT mediates robust overexpression and increases circulating levels of FGF21 and has the following benefits:
improved locomotor activity and neuromuscular performance
reduced anxiety-like behavior

In Example 3, intramuscular administration of AAV1-CMV-moFGF21 mediates robust overexpression and increases circulating levels of FGF21 and has the following benefits:
improved coordination, balance, neuromuscular performance, strength and locomotor activity
reduced anxiety-like behavior
improved cognitive performance, memory, learning and exploratory capacity

In Example 4, intra-CSF administration of AAV1-CAG-moFGF21 mediates robust overexpression and has the following benefits:
improved locomotor activity
reduced anxiety-like behavior
improved cognitive performance, memory and exploratory capacity

In Example 5, intra-CSF administration of AAV9-CAG-moFGF21-dmiRT mediates robust overexpression and has the following benefits:
decreased neuroinflammation indicating improvement of depression

In Examples 8 and 9, intramuscular administration of AAV1-CMV-moFGF21 is shown to mediate a positive therapeutic effect in SAMP8 mice (widely used mouse model of senescence with age-related brain pathologies such as neuroinflammation) and in 3×Tg-AD mice (Alzheimer disease model).

In Example 10, intramuscular administration of AAV1-CMV-moFGF21 is shown to lead to improved coordination, balance and motor learning as well as short- and long-term memory.

In Example 11, it was shown that intramuscular administration of AAV1-CMV-moFGF21 inhibited neurodegeneration and cognitive decline by improvement of mitochondrial function, increase of glucose metabolism and autophagia, diminution of oxidative and ER stress, and amelioration of cholesterol homeostasis and synaptic function in cortex and hippocampus of old mice.

In Examples 12 and 13, intra-CSF administration of AAV1-CAG-moFGF21 improved the neuromuscular and cognitive decline associated with diabetes and obesity and improved neuromuscular performance and enhanced learning and short and long-term memory in old mice.

### General Procedures to the Examples

Subject Characteristics

Male SAMP8/TaHsd (SAMP8), male and female C57Bl/6J mice, male BKS.Cg-+Lepr^{db}/+Lepr^{db}/OlaHsd (db/db) and male BKS.Cg-m+/+Lepr^{db}/OlaHsd (db/+, lean) mice, male SAMR1/TaHsd (SAMR1) mice, male 3×Tg-AD (B6; 129Tg(APPSwe,tauP301L)1Lfa Psen1^{tm1Mpm}) and male B6129SF2/J were used. Mice were fed ad libitum with a standard diet (2018S Teklad Global Diets®, Harlan Labs, Inc., Madison, Wis., US) or a high fat diet (TD.88137 Harlan Teklad Madison, Wis., US) and kept under a light-dark cycle of 12 h (lights on at 8:00 a.m.) and stable temperature (22° C.±2). When stated, mice were fasted for 16 h. For tissue sampling, mice were anesthetized by means of inhalational anesthetic isoflurane (IsoFlo®, Abbott Laboratories, Abbott Park, Ill., US) and decapitated. Tissues of interest were excised and kept at −80° C. or with formalin until analysis. All experimental procedures were approved by the Ethics Committee for Animal and Human Experimentation of the Universitat Autònoma de Barcelona.

Recombinant AAV Vectors

Single-stranded AAV vectors of serotype 1 or 8 or 9 were produced by triple transfection of HEK293 cells according to standard methods (Ayuso, E. et al., 2010. Curr Gene Ther. 10(6):423-36). Cells were cultured in 10 roller bottles (850 cm², flat; Corning™, Sigma-Aldrich Co., Saint Louis, Mo., US) in DMEM 10% FBS to 80% confluence and co-transfected by calcium phosphate method with a plasmid carrying the expression cassette flanked by the AAV2 ITRs, a helper plasmid carrying the AAV2 rep gene and the AAV of serotypes 1 or 8 cap gene, and a plasmid carrying the adenovirus helper functions. Transgenes used were: the murine codon-optimized FGF21 coding-sequence driven by 1) the cytomegalovirus (CMV) early enhancer/chicken beta actin (CAG) promoter; 2) the cytomegalovirus (CMV) early enhancer/chicken beta actin (CAG) promoter with the addition of four tandem repeats of the miRT122a sequence (5′CAAACACCATTGTCACACTCCA3′) (SEQ ID NO:12) and four tandems repeats of the miRT1 sequence (5′TTACATACTTCTTTACATTCCA3′) (SEQ ID NO:13) cloned in the 3′ untranslated region of the expression cassette; or 3) the CMV promoter. A Noncoding plasmid carrying the CMV promoter was used to produce null vectors. AAV were purified with an optimized method based on a polyethylene glycol precipitation step and two consecutive cesium chloride (CsCl) gradients. This second-generation CsCl-based protocol reduced empty AAV capsids and DNA and protein impurities dramatically (Ayuso, E. et al., 2010. Curr Gene Ther. 10(6):423-36). Purified AAV vectors were dialyzed against PBS, filtered and stored at −80° C. Titers of viral genomes were determined by quantitative PCR following the protocol described for the AAV2 reference standard material using linearized plasmid DNA as standard curve (Lock M, et al., Hum. Gene Ther. 2010; 21:1273-1285). The vectors were constructed according to molecular biology techniques well known in the art.

In Vivo Intra-eWAT Administration of AAV Vectors

Mice were anesthetized with an intraperitoneal injection of ketamine (100 mg/kg) and xylazine (10 mg/kg). A laparotomy was performed in order to expose the epididymal white adipose tissue. AAV vectors were resuspended in PBS with 0.001% Pluronic® F68 (Gibco) and injected directly into the epididymal fat pad. Each epididymal fat pad was injected twice with 50 μL of the AAV solution (one injection close to the testicle and the other one in the middle of the fat pad). The abdomen was rinsed with sterile saline solution and closed with a two-layer approach.

Intramuscular Administration of AAV Vectors

Mice were anesthetized with an intraperitoneal injection of ketamine (100 mg/kg) and xylazine (10 mg/kg). Hind limbs were shaved and vectors were administered by intramuscular injection in a total volume of 180 μl divided into six injection sites distributed in the quadriceps, gastrocnemius, and tibialis cranealis of each hind limb.

In Vivo Intra-CSF Administration of AAV Vectors

Mice were anesthetized with an intraperitoneal injection of ketamine (100 mg/kg) and xylazine (10 mg/kg), and the skin of the posterior part of the head, from behind the ears to approximately between the scapulas, was shaved and rinsed with ethanol. Mice were held in prone position, with the head at a slightly downward inclination. A 2-mm rostro-caudal incision was made to introduce a Hamilton syringe at an angle of 45-55° into the cisterna magna, between the occiput and the Cl-vertebra and 5 μl of vector dilution was administered. Given that the CNS is the main target compartment for vector delivery, mice were dosed with the same number of vector genomes/mouse irrespective of body weight (5×10⁹, 1×10¹⁰ and 5×10¹⁹ vg/mice).

RNA Analysis

Total RNA was obtained from adipose depots or skeletal muscle by using QIAzol Lysis Reagent (Qiagen NV, Venlo, NL) or Tripure isolation reagent (Roche Diagnostics Corp., Indianapolis, Ind., US), respectively, and RNeasy Lipid Tissue Minikit (Qiagen NV, Venlo, NL). In order to eliminate the residual viral genomes, total RNA was treated with DNAseI (Qiagen NV, Venlo, NL). For RT-PCR, 1 μg of RNA samples was reverse-transcribed using Transcriptor First Strand cDNA Synthesis Kit (04379012001, Roche, California, USA). Real-time quantitative PCR was performed in a SmartCyclerII® (Cepheid, Sunnyvale, USA) using EXPRESS SYBRGreen qPCR supermix (Invitrogen™, Life Technologies Corp., Carlsbad, Calif., US). Data was normalized with Rplp0 values and analyzed as previously described (Pfaffl, M., Nucleic Acids Res. 2001; 29(9):e45). Total RNA was obtained from hypothalamus, cortex, hippocampus, cerebellum and olfactory bulb using Tripure isolation reagent (Roche Diagnostics Corp., Indianapolis, Ind., US), and RNeasy Mini Kit or RNeasy Micro Kit for hippocampus samples (Qiagen NV, Venlo, NL). In order to eliminate the residual viral genomes, total RNA was treated with DNAseI (Qiagen NV, Venlo, NL). For RT-PCR analysis, 1 μg of RNA samples was reverse-transcribed using Transcriptor First Strand cDNA Synthesis Kit (04379012001, Roche, California, USA). Real-time quantitative PCR was performed in a SmartCyclerII® (Cepheid, Sunnyvale, USA) using TB Green Premix Ex TaqII (Takara Bio Europe, France). Data was normalized with Rplp0 values and analyzed as previously described (Pfaffl, M., Nucleic Acids Res. 2001; 29(9):e45).

An overview of the primers used is shown below:

moFgf21-Fw:
(SEQ ID NO: 47)
5′-CCTTTCCTGGTCGCCTCTTAG-3′
moFgf21-Rv:
(SEQ ID NO: 48)
5′-GTTCCACCATGCTCAGAGGG-3′
Gfap-Fw:
(SEQ ID NO: 49)
5′-ACAGACTTTCTCCAACCTCCAG-3′
Gfap-Rv:
(SEQ ID NO: 50)
5′-CCTTCTGACACGGATTTGGT-3′
S100b-Fw:
(SEQ ID NO: 51)
5′-AACAACGAGCTCTCTCACTTCC-3′
S100b-Rv:
(SEQ ID NO: 52)
5′-CGTCTCCATCACTTTGTCCA-3′
Aif1-Fw:
(SEQ ID NO: 53)
5′-TGAGCCAAAGCAGGGATTTG-3′
Aif1-Rv:
(SEQ ID NO: 54)
5′-TCAAGTTTGGACGGCAGATC-3′
Nfkb-Fw:
(SEQ ID NO: 55)
5′-GACCACTGCTCAGGTCCACT-3′
Nfkb-Rv:
(SEQ ID NO: 56)
5′-TGTCACTATCCCGGAGTTCA-3′
Il1b-Fw:
(SEQ ID NO: 57)
5′-ATGAAGGGCTGCTTCCAAAC-3′
Il1b-Rv:
(SEQ ID NO: 58)
5′-ATGTGCTGCTGCGAGATTTG-3′
Il6-Fw:
(SEQ ID NO: 59)
5′-TCGCTCAGGGTCACAAGAAA-3′
Il6-Rv:
(SEQ ID NO: 60)
5′-CATCAGAGGCAAGGAGGAAAAC-3′
Rplp0-Fw:
(SEQ ID NO: 61)
5′-ACTGGTCTAGGACCCGAGAA-3′
Rplp0-Fw:
(SEQ ID NO: 62)
5′-TCCCACCTTGTCTCCAGTCT-3′
Ccl19-Fw:
(SEQ ID NO: 69)
5′-GCGGGCTCACTGGGGCACAC-3′
Ccl19-Rv:
(SEQ ID NO: 70)
5′-TGGGAAGGTCCAGAGAACCAG-3′
Ppargc1a-Fw:
(SEQ ID NO: 71)
5′-TTTGGCCGACGACACGACTTTTC-3′
Ppargc1a-Rv:
(SEQ ID NO: 72)
5′-TTGTGTTGGGCGAGAGAAAG-3′
Ppargc1b-Fw:
(SEQ ID NO: 73)
5′-AGAAGCGCTTTGAGGTGTTC-3′
Ppargc1b-Rv:
(SEQ ID NO: 74)
5′-GGTGATAAAACCGTGCTTCTGG-3′
Atp5f1a-Fw:
(SEQ ID NO: 75)
5′-TCTCGGCCAGAGACTAGGAC-3′
Atp5f1a-Rv.
(SEQ ID NO: 76)
5′-GCACTTGCACCAATGAATTT-3′
Mt-co1-Fw:
(SEQ ID NO: 77)
5′-ATGAGCAAAAGCCCACTTCG-3′
Mt-co1-Rv:
(SEQ ID NO: 78)
5′-ACCGTGGAGATTTGGTCCAG-3′
Cox6-Fw:
(SEQ ID NO: 79)
5′-AGTCCCTCTGTCCCGTGTC-3′
Cox6-Rv:
(SEQ ID NO: 80)
5′-ATATGCTGAGGTCCCCCTTT-3′
Cox5a-Fw:
(SEQ ID NO: 81)
5′-CTCGTCAGCCTCAGCCAGT-3′
Cox5a-Rv:
(SEQ ID NO: 82)
5′-TAGCAGCGAATGGAACAGAC-3′
Sod1-Fw:
(SEQ ID NO: 83)
5′-TACACAAGGCTGTACCAGTGC-3′
Sod1-Rv:
(SEQ ID NO: 84)
5′-TTTCCAGCAGTCACATTGCC-3′
Nrf2-Fw:
(SEQ ID NO: 85)
5′-AGTCGCTTGCCCTGGATATC-3′
Nrf2-Rv:
(SEQ ID NO: 86)
5′-TGCCAAACTTGCTCCATGTC-3′
Cat-Fw:
(SEQ ID NO: 87)
5′-TGTGCATGCATGACAACCAG-3′
Cat-Rv:
(SEQ ID NO: 88)
5′-GCACTGTTGAAGCGTTTCAC-3′
Gapdh-Fw:
(SEQ ID NO: 89)
5′-CCTTCCGTGTTCCTACCC-3′
Gapdh-Rv:
(SEQ ID NO: 90)
5′-CAACCTGGTCCTCACTGTAG-3′
Hkl-Fw:
(SEQ ID NO: 91)
5′-ACGGTCAAAATGCTGCCTTC-3′
Hkl-Rv:
(SEQ ID NO: 92)
5′-AATCGTTCCTCCGAGATCCA-3′
Pfkp-Fw:
(SEQ ID NO: 93)
5′-TGTGTCTGAAGGAGCAATCG-3′
Pfkp-Rv:
(SEQ ID NO: 94)
5′-GGCCAAAATCCTGTCAAATG-3′
Gpd1-Fw:
(SEQ ID NO: 95)
5′-AGACACCCAACTTTCGCATC-3′
Gpd1-Rv:
(SEQ ID NO: 96)
5′-TATTCTTCAAGGCCCCACAG-3′
Gpd2-Fw:
(SEQ ID NO: 97)
5′-TTGCCTTGGGAGAAGATGAC-3′
Gpd2-Rv:
(SEQ ID NO: 98)
5′-AGTTCCGCACTTCATTCAGG-3′
Pkm-Fw:
(SEQ ID NO: 99)
5′-GCTTTGCATCTGATCCCATT-3′
Pkm-Rv:
(SEQ ID NO: 100)
5′-AGTCCAGCCACAGGATGTTC-3′
Syp-Fw:
(SEQ ID NO: 101)
5′-ACATGGACGTGGTGAATCAG-3′
Syp-Rv:
(SEQ ID NO: 102)
5′-AAGATGGCAAAGACCCACTG-3′
Gria1-Fw:
(SEQ ID NO: 103)
5′-CCATGCTGGTTGCCTTAATC-3′
Gria1-Rv:
(SEQ ID NO: 104)
5′-CCGTATGGCTTCATTGATGG-3′
Gria2-Fw:
(SEQ ID NO: 105)
5′-AAGGGCGTGTAATCCTTGAC-3′
Gria2-Rv:
(SEQ ID NO: 106)
5′-TTTCAGCAGGTCTCCATCAG-3′
Grin1-Fw:
(SEQ ID NO: 107)
5′-TGACTACCCGAATGTCCATC-3′
Grin1-Rv:
(SEQ ID NO: 108)
5′-TTGTAGACGCGCATCATCTC-3′
Grin2a-Fw:
(SEQ ID NO: 109)
5′-TGTGAAGAAGTGCTGCAAGG-3′
Grin2a-Rv:
(SEQ ID NO: 110)
5′-CGCCTATCATTCCATTCCAC-3′
Grin2b-Fw:
(SEQ ID NO: 111)
5′-TTGGTGAGGTGGTCATGAAG-3′
Grin2b-Rv:
(SEQ ID NO: 112)
5′-TGCGTGATACCATGACACTG-3′
Sqstm1-Fw:
(SEQ ID NO: 113)
5′-TGCTGGCGGCTTTACATTTG-3′
Sqstm1-Rv:
(SEQ ID NO: 114)
5′-CAGAAGCAGAGAAGGAAAAGCC-3′
Atg5-Fw:
(SEQ ID NO: 115)
5′-AGATGGACAGCTGCACACAC-3′
Atg5-Rv:
(SEQ ID NO: 116)
5′-TTGGCTCTATCCCGTGAATC-3′
Atf4-Fw:
(SEQ ID NO: 117)
5′-ATGATGGCTTGGCCAGTG-3′
Atf4-Rv:
(SEQ ID NO: 118)
5′-CCATTTTCTCCAACATCCAATC-3′
Bip-Fw:
(SEQ ID NO: 119)
5′-CTGAGGCGTATTGGGAAG-3′
Bip-Rv:
(SEQ ID NO: 120)
5′-TCATGACATTCAGTCCAGCAA-3′
Cyp46a1-Fw:
(SEQ ID NO: 121)
5′-TCGTTGAACGTCTCCATCAG-3′
Cyp46a1-Rv:
(SEQ ID NO: 122)
5′-TTTGGGGAGAGACTGTTTGG-3′

Analysis of mRNA Expression with Microarrays

cDNA synthesis and array hybridization. For the analysis of the mRNA expression Affymetrix Clariom S Mouse microarray (Affymetrix, Thermo Fisher Scientific, Waltham, Mass., USA) was used. Approximately 300 ng of total RNA were processed using the GeneChip WT Plus Reagent kit (Affymetrix, Thermo Fisher Scientific, Waltham, Mass., USA) following the manufacturer instructions and hybridized to Affymetrix Clariom S Mouse microarray plates. The Affymetrix GeneChip Hybridization, Wash, and Stain kit were used for array processing. The chips were subsequently scanned with an Affymetrix GeneChip Scanner 3000.

Array quality control and normalization. The Expression Console™ Software (Affymetrix, Thermo Fisher Scientific, Waltham, Mass., USA) was also used to perform quality control of microarrays and to normalize the data of all the microarrays. RMA algorithm was used to perform background correction, log 2 transformation, and quantile normalization to allow the comparison of values across microarrays. Afterwards, Affymetrix Transcriptome Analysis Console Software (Affymetrix, Thermo Fisher Scientific, Waltham, Mass., USA) was used to annotate and compare FGF21 treated brain samples vs Null treated brain samples to generate a list of genes with computed fold change and p-value.

Measurement of FGF21 Circulating Levels

Circulating levels of FGF21 were determined by quantitative sandwich enzyme immunoassay Mouse/Rat FGF-21 ELISA kit (MF2100, R&Dsystems, Abingdon, UK).

Amyloid Beta Extraction and Quantification

Dissected cortex was homogenized using a sonicator (Sonics, Vibra-Cell, Newtown, USA) in cold T-PER buffer (ThermoScientific, Rockford, Ill., USA) supplemented with a protease inhibitor cocktail (Complete EDTA-free, Roche, Mannheim, Germany). After a brief sonication the samples were centrifuged at 100,000×g at 4° C. for 1 h in an Ultracentrifugue (Optima XPN-100, Beckman Coulter, Brea, Calif., USA) using a SW-55Ti rotor. The supernatant was labelled as the soluble fraction. The pellet was re-suspended in 70% formic acid solution. Sonication and centrifugation steps were repeated and the supernatant was recovered and dried for 4 hours in a vacuum concentrator (Savant SpeedVac DNA130 Concentrator, ThermoFischer Scientific). The dried formic extract was re-suspended in DMSO and labeled as insoluble fraction. All fractions were immediately stored at −80° C. until further use.

Aβ40 levels were quantified in the insoluble fraction by ELISA following the protocol recommended by the manufacturer (Human Aβ40 ELISA kit, Invitrogen, ref. KHB3481). Data were normalized to the total amount of protein in each sample (Pierce BCA Protein Assay Kit, Thermo Scientific, ref. 23225).

Open Field Test

The open field test was performed between 9:00 am and 1:00 pm as previously reported (Haurigot et al, 2013). Briefly, animals were placed in a corner of a white plastic walls and floor box (45×45×40 cm). For C57Bl/6J mice motor and exploratory activities were evaluated during the first 6 minutes using a video tracking system (SMART Junior; Panlab). For db/db mice and their control groups, mice were first habituated for 5 minutes in the open field arena. Then, they were placed for 5 minutes in the home cage and afterwards, they were placed again in the open field arena and motor and exploratory activities were evaluated during the first 12 minutes.

Novel Object Recognition Test

The novel object recognition tests were conducted in the open field box. Open-field test was used to acclimatize the mice to the box. The next day, to conduct the first trial, two identical objects (A and B) were placed in the upper right and upper left quadrants of the box, and then mice were placed backwards to both objects. After 10 min of exploration, mice were removed from the box, and allowed for 10 min break. In the second trial, one of the identical objects (A and B) was replaced with object C (new object). Mice were then put back into the box for a further 10 minutes of exploration for the short-term memory trial. For the long-term memory trial, the day after, the object C was replaced by a new object (D), allowing the mice to explore objects A and D for a further 10 minutes. The amount of time animals spent exploring the novel object was recorded and evaluated using a video tracking system (SMART Junior; Panlab). The evaluation of novel object recognition test memory was expressed as a percentage of the discrimination ratio calculated according to the following formula: Discrimination ratio (%)=(N−F)/(N+F)×100%, where N represents the time spent in exploring the new object and F represents the time spent in exploring the same object.

Rotarod Test

Mice were placed on a rotating rod (Panlab, Barcelona, Spain), spinning at 4 RPM. Lane width, 50 mm; rod diameter, 30 mm. Once stabilized, mice were subjected to an incrementally increasing speed of x RPM per x s. The first day of the experiment was used to train the animals in the use of the device. Each animal underwent 3 trials. The length of time that the mice managed to remain on the rod was recorded. Then, animals underwent 1 day resting and the third day, mice took 3 more trials on the rod. The average of 3 trials was analyzed. For evaluation of motor learning, performance in each individual trial was analyzed.

Grip Strength Test

A grip strength test meter (Panlab, Barcelona, Spain) was used to assess forelimb grip strength. The grip strength meter was positioned horizontally and mice were held by the tail and lowered towards the apparatus. Animals were allowed to grasp the metal bar with their front paws and were then pulled backwards in the horizontal plane. The force applied to the bar just before it lost grip was recorded as the peak tension. The average of 3 trials was analyzed.

Hang Wire Test

The wire hang test was conducted using a 55 cm wide 2-mm thick metallic wire which was secured to two vertical stands. The wire was maintained 35 cm above a layer of bedding material to prevent injury to the animal when it falls down. Mice, handled by the tail, were allowed to grasp the middle of the wire with its fore limbs. The time until mice fell down was measured. Mice that reached the limit suspension time of 180 seconds, independent on the trial number, were allowed to stop the experiment, while the others were directly retested for a maximum of three trials (a 30 seconds recovery period was used between trials).

Barnes Maze Test

The Barnes maze test consisted of an elevated circular platform with a 20 evenly-spaced holes around the perimeter. An escape box is mounted under one hole while the remaining 19 holes are left covered. During training and test, aversive stimulus such as bright light (more than 1000 lumens), open space and noise (more than 90 db) served as a motivation factor to induce escape behavior. Barnes maze was conducted in an empty room and visual cues in the walls were used as a reference. During the first day animals were acclimated during 1 minute in the scape box followed by 140 seconds in the open platform. Once all animals were acclimated, escape box was moved to another hole in the Barnes maze where it was maintained for the duration of the trainings. In the first training, mice were placed inside a PVC tube during 15 seconds in the middle of the Barnes maze and then PVC was released and animals were free to explore the platform and find the escape box for 140 seconds. If they found the correct hole and entered the escape box, animals remained inside for 30 seconds, if not the animals were guided to the scape box. In the following days (2, 3 and 4), two trainings per day were assessed as the first training. The last day (day 5), the scape box was removed, and a probe trial was conducted to assess memory for 180 seconds. The amount of time that animals spent exploring the Barnes maze was recorded and evaluated using a video tracking system (SMART Junior; Panlab). The time that animals spend until they found the scape box was calculated as a measure of memory.

Elevated Plus Maze

The elevated plus maze test was conducted in an apparatus which consists of open and closed arms, crossed in the middle, and a center area. The structure was elevated 90-100 cm from the floor. During the test, mice were placed in the center area and were allowed to move freely between arms for 5 minutes. The amount of time that animals spent exploring the open and closed arms was recorded and evaluated using a video tracking system (SMART Junior; Panlab). The number of entries into the open arms and the time spent in the open arms are used as index of open space-induced anxiety in mice.

Statistical Analysis

All values are expressed as mean±SEM. Data were analyzed by one-way ANOVA with Tukey's post hoc correction, except for those parameters involving comparison of only two experimental groups, in which case an unpaired Student's t-test was used. Differences were considered significant when P<0.05.

### Example 1. Improved Neuromuscular Performance and Cognition and Decreased Neurodegeneration in Old Mice Treated with AAV Vectors Encoding FGF21

To evaluate whether genetic engineering of the skeletal muscle with FGF21 may exert therapeutic benefit in old animals, 13.5-month-old male C57Bl6 mice were administered intramuscularly with 3×10¹¹ viral genomes (vg) of AAV vectors of serotype 1 encoding a murine codon-optimized FGF21 coding sequence (moFGF21) under the control of the CMV promoter (AAV1-CMV-moFGF21). Age-matched control animals were treated with the same dose of AAV1-CMV-Null vectors. Untreated cohorts of younger mice served as additional control groups. All experimental groups were fed with a chow diet.

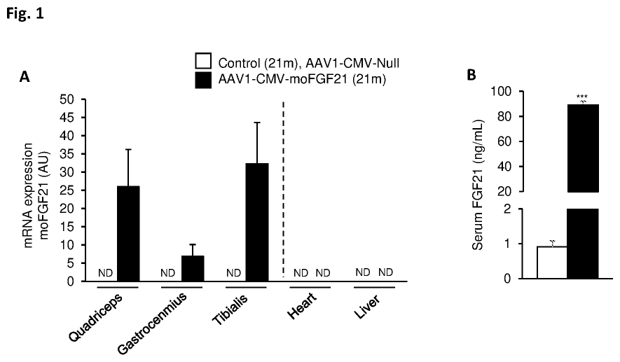

AAV1-CMV-moFGF21 treated mice showed overexpression of codon-optimized FGF21 in the three injected muscles but not in off-target tissues such as the liver and heart (FIG. 1A). Skeletal muscle overexpression of FGF21 resulted in increased secretion of FGF21 into the bloodstream (FIG. 1B).

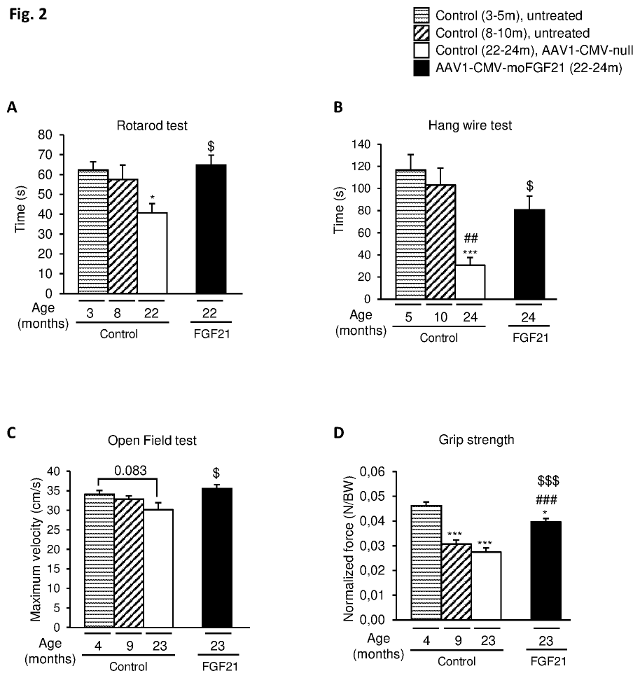

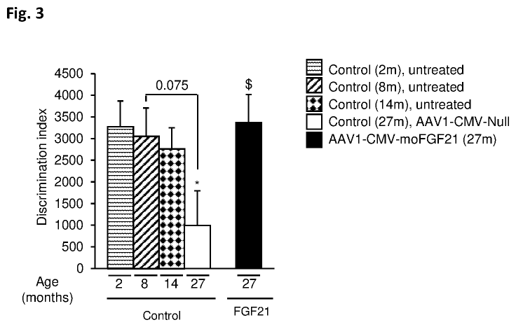

Treatment of old mice with AAV1-CMV-moFGF21 vectors improved coordination and balance. Noticeably, no differences between 22-month-old AAV1-CMV-moFGF21-treated mice and 3-month-old untreated mice were observed (FIG. 2A). To further study skeletal muscle function and coordination, the hang wire test was performed. Old mice treated with AAV1-CMV-moFGF21 showed improved neuromuscular performance in comparison with mice administered with Null vectors (FIG. 2B). The open field test revealed that physical activity levels decreased with age (FIG. 2C). AAV-CMV-moFGF21-treated mice showed significantly increased activity levels, making the activity levels of 23-month-old AAV-FGF21-treated mice similar to that of 4-month-old untreated mice (FIG. 1C). In agreement with previous reports (Wenz, T. et al. 2009. Proc Natl Acad Sci USA 106 (48), 20405-10), the grip strength test evidenced loss of muscular strength associated with aging (FIG. 2D). AAV-CVM-moFGF21-treated mice showed a significant improvement of this parameter in comparison with AAV1-CMV-Null age-matched counterparts, being grip strength of the former mice slightly reduced in comparison with that of 4-month-old mice (FIG. 2D). Moreover, by 27 months of age, mice treated with FGF21-encoding vectors performed markedly better in the novel object recognition test than the age-matched cohort treated with AAV1-CMV-Null vectors and had a recognition index equivalent to that of 2-month-old animals (FIG. 3). All these results suggest that treatment with AAV1-CMV-moFGF21 vectors improved neuromuscular performance, enhanced learning and normalized memory in old mice.

To gain insight into the molecular mechanisms underlying the AAV-FGF21 mediated improvement of cognition, RNA from brain of old mice treated with AAV1-CMV-moFGF21 or AAV1-CMV-Null vectors was obtained, and transcriptomic analysis was performed using the Affymetrix Clariom S Mouse microarray technology. Pre-processing of the data was done using the Affymetrix Expression Console. Afterwards, the Affymetrix Transcriptome Analysis Console was used to compare brain samples from old mice treated with AAV1-CMV-moFGF21 or AAV1-CMV-Null vectors to generate a list of genes with computed fold change and p-value. Gene Set Enrichment Analysis (GSEA) was performed for interpreting transcriptomic data obtained from microarray analysis. This method relies on gene sets, that is, groups of genes that share common features based on prior biological knowledge, e.g., biological function, biological pathway, or cellular compartment (Subramanian, A. et al., 2005). These sets contain a variable number of genes (Size of gene set) and were retrieved from several databases such as Hallmark, KEGG, Reactome, or Gene Ontology (GO) and then overrepresentation analysis was computed. The goal of GSEA is to determine whether members of a gene set tend to correlate with treated vs non-treated samples. The degree to which a set is overrepresented was calculated and normalized to account for the size of the set, yielding a normalized enrichment score (NES), and the associated p-value to account for statistical significance.

In agreement with previous reports describing improvement of neurodegeneration and cognitive decline in animals treated with recombinant FGF21 protein mainly due to enhanced mitochondrial function and diminution of oxidative stress (Yu, Y. et al., 2015; Wang, X-M. et al., 2016; Sa-nguanmoo P. et al 2016; Sa-nguanmoo P. et al 2018; Chen S. et al., 2019; Amiri M. et al., 2018), the GSEA revealed that pathways related to oxidative phosphorylation, respiratory electron transport, uncoupling protein-mediated thermogenesis, reactive oxygen species, mitochondrial complexes and components, cristae formation and mitochondrial transmembrane transport were enriched in old-animals treated with AAV1-CMV-moFGF21 vectors in comparison with mice receiving AAV1-CMV-Null vectors (Table 1). The data thus indicates that FGF21 gene therapy inhibits neurodegeneration by improvement of mitochondrial function and diminution of oxidative stress.

TABLE 1
Enriched Gene Sets relevant to oxidative and mitochondrial
metabolism obtained from GSEA analysis

| | | | NES | |
|---|---|---|---|---|
| | | Size (# | (normalized | |
| | | genes in | enrichment | |
| Enriched set | Database | the set) | score) | p-value |
| Oxidative | hallmark | 191 | 2.28 | <0.001 |
| phosphorylation | | | | |
| Reactive oxygen | hallmark | 45 | 1.62 | 0.015 |
| species pathway | | | | |
| Oxidative | KEGG | 110 | 2.19 | <0.001 |
| phosphorylation | | | | |
| Respiratory electron | REACTOME | 103 | 2.32 | <0.001 |
| transport atp | | | | |
| synthesis by | | | | |
| chemiosmotic | | | | |
| coupling and heat | | | | |
| production by | | | | |
| uncoupling proteins | | | | |
| Respiratory electron | REACTOME | 83 | 2.18 | <0.001 |
| transport | | | | |
| Mitochondrial | REACTOME | 92 | 2.1 | <0.001 |
| translation | | | | |
| Complex i | REACTOME | 48 | 2.06 | <0.001 |
| biogenesis | | | | |
| The citric acid | REACTOME | 152 | 2.02 | <0.001 |
| tca cycle and | | | | |
| respiratory electron | | | | |
| transport | | | | |
| Intrinsic component | GO cellular | 38 | 1.78 | <0.001 |
| of mitochondrial | component | | | |
| inner membrane | | | | |
| Intrinsic component | GO cellular | 65 | 1.75 | 0.004 |
| of mitochondrial | component | | | |
| membrane | | | | |
| Mitochondrial | GO cellular | 237 | 1.69 | 0.027 |
| protein complex | component | | | |
| Inner mitochondrial | GO cellular | 115 | 1.68 | 0.018 |
| membrane protein | component | | | |
| complex | | | | |
| Proton transporting | GO cellular | 20 | 1.65 | <0.001 |
| two sector atpase | component | | | |
| complex proton | | | | |
| transporting domain | | | | |
| Organelle inner | GO cellular | 486 | 1.65 | 0.019 |
| membrane | component | | | |
| Proton transporting | GO cellular | 48 | 1.64 | <0.001 |
| two sector atpase | component | | | |
| complex | | | | |
| Inner mitochondrial | GO biological | 43 | 1.77 | <0.001 |
| membrane | process | | | |
| organization | | | | |
| Mitochondrial | GO biological | 90 | 1.73 | <0.001 |
| transmembrane | process | | | |
| transport | | | | |
| Cristae formation | GO biological | 30 | 1.69 | <0.001 |
| | process | | | |
| Establishment | GO biological | 17 | 1.67 | 0.022 |
| of protein | process | | | |
| localization to | | | | |
| mitochondrial | | | | |
| membrane | | | | |

### Example 2. Reversal of Hypoactivity and Anxiety- and Depression-Like Symptoms in HFD-Fed Male Mice Treated with AAV Vectors Encoding FGF21

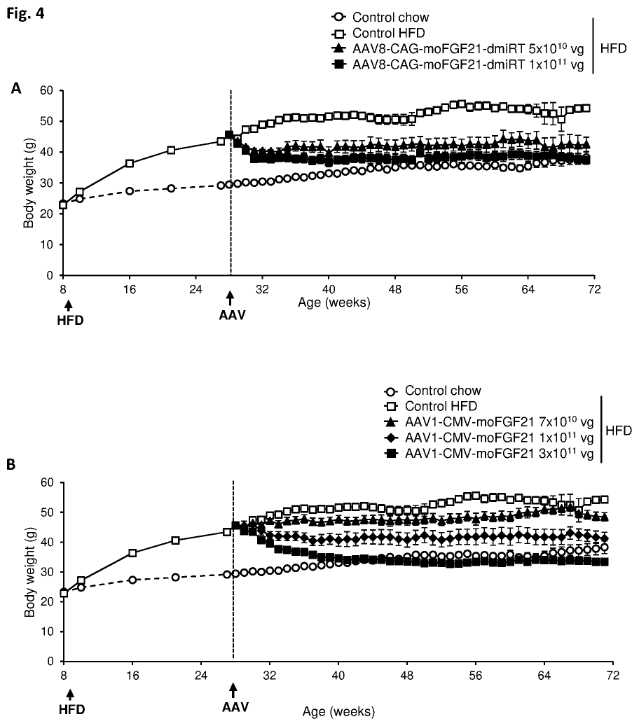

We evaluated the therapeutic potential of the AAV-mediated genetic engineering of adipose tissue or skeletal muscle with FGF21 to revert obesity- and diabetes-associated anxiety and decreased neuromuscular performance. To this end, 10-week-old male C57Bl6 mice were fed a HFD for 18 weeks. During these first 4 months of follow-up, while the weight of chow-fed animals increased by 25%, animals fed a HFD became obese (91% body weight gain) (FIG. 4A-B). Obese animals were then administered intra-eWAT (eWAT: epididymal white adipose tissue) with 5×10¹⁰ vg or 1×10¹¹ vg of AAV8 vectors encoding a murine codon-optimized FGF21 coding sequence under the control of the CAG ubiquitous promoter which included target sites of miR122a and miR1 (AAV8-CAG-moFGF21-dmiRT). Another cohort of obese mice was administered intramuscularly (im) with AAV1-CMV-moFGF21 vectors at 3 different doses: 7×10¹⁰, 1×10¹¹, and 3×10¹¹ vg/mouse. After AAV administration, AAV-treated mice were maintained on HFD for about 1 year, i.e. up to 16.5 months of age. As controls, untreated chow- and HFD-fed C57Bl6 mice were used.

Animals treated with 5×10¹⁰ vg or 1×10¹¹ vg of AAV8-CAG-moFGF21-dmiRT vectors initially lost 14% and 25% of body weight, respectively, and continued to progressively lose weight (FIG. 4A). Indeed, body weight of HFD-fed mice treated with 1×10¹¹ vg of AAV8-CAG-moFGF21-dmiRT was similar to body weight of chow-fed animals towards the end of the study (˜14.5 months) (FIG. 4A).

A clear dose-dependent loss of body weight was observed in the groups treated with AAV1-CMV-moFGF21. The lowest dose of vector did not counteract the weight gain associated to HFD-feeding, although the mean weight of these animals was always lower than that of control HFD-fed mice (FIG. 4B). Animals treated with 1×10¹¹ vg AAV1-CMV-moFGF21 initially lost 18% of body weight and continued to progressively lose weight (FIG. 4B). Body weight of these mice was similar to the weight of chow-fed animals towards the end of the study (˜14.5 months) (FIG. 4B). Upon administration of 3×10¹¹ vg AAV1-CMV-FGF21, HFD-fed mice initially lost 34% of body weight and also experienced progressive loss of body weight, which by the end of the study (16.5 months of age) was lower than weight of chow-fed animals and slightly increased in comparison to the weight documented before the initiation of the HFD-feeding (FIG. 4B).

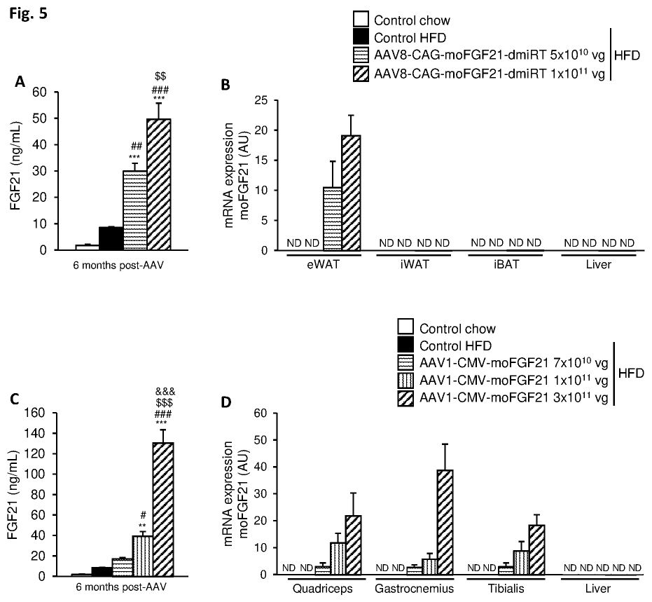

Animals treated intra-eWAT with 5×10¹⁰ or 1×10¹¹ vg of AAV8-CAG-moFGF21-dmiRT vectors showed high levels of FGF21 in the bloodstream (FIG. 5A) mediated by adipose-specific overexpression of FGF21 (FIG. 5B). Similarly, HFD-fed mice treated with AAV1-CMV-FGF21 vectors showed a marked increase in circulating FGF21 (FIG. 5C), which was parallel to high levels of expression of vector-derived FGF21 in the 3 injected muscles (FIG. 5D). This combination of vector serotype, promoter and route of administration did not lead to expression of the transgene in off-target tissues such as the liver (FIG. 5D).

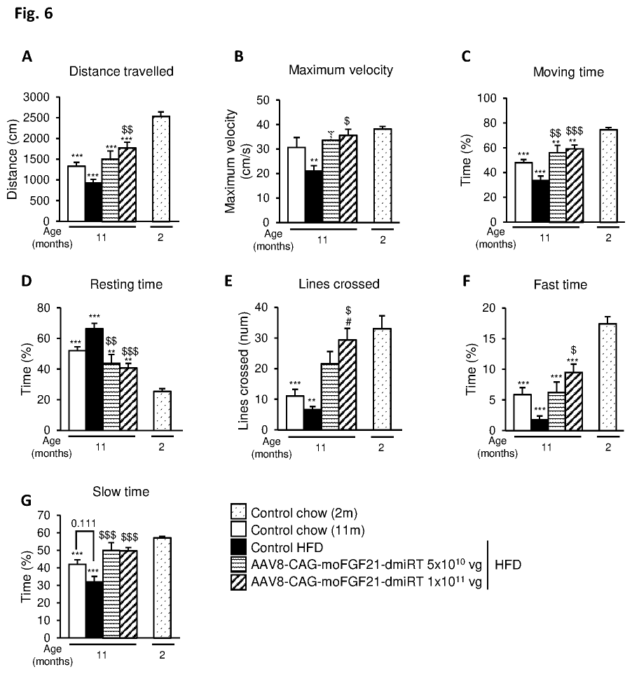

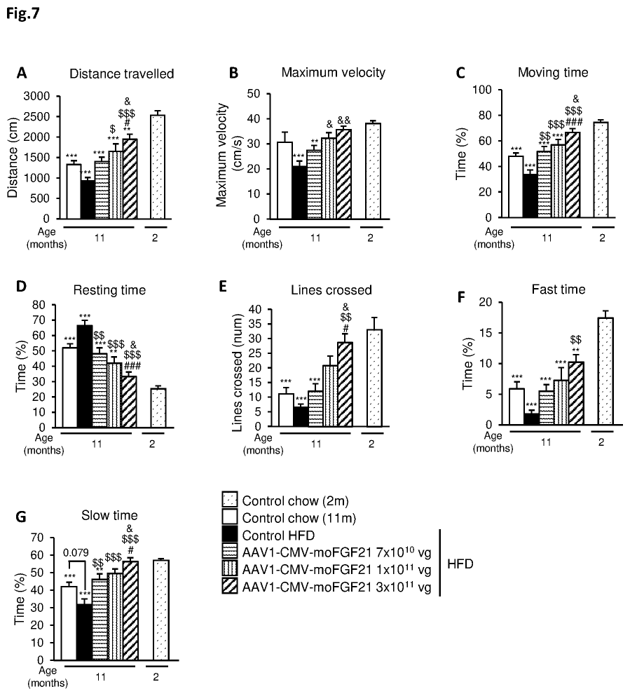

Treatment with AAV8-CAG-moFGF21-dmiRT or AAV1-CMV-moFG F21 vectors mediated effects on locomotor activity. In contrast to the hypoactivity observed in the open field test in the untreated animals fed a HFD, mice treated with 5×10¹⁰ vg or 1×10¹¹ vg of AAV8-CAG-moFGF21-dmiRT showed the same degree of spontaneous locomotor activity than chow-fed animals (FIG. 6). Eleven month old AAV8-CAG-moFGF21-dmiRT-treated animals travelled more distance, moved more time and at higher velocity, rested less time and spent more time doing slow and fast movements than untreated HFD-fed controls (FIG. 6A-G). Similar observations were made in mice treated im with 1×10¹¹ and 3×10¹¹ vg of AAV1-CMV-moFGF21 (FIG. 7). These results suggest improved neuromuscular performance in HFD-fed mice treated with FGF21-encoding AAV vectors. These results also indicate a reduction in behavior that is typically characterized as depression-like behavior in the open-field test, such as total distance travelled (see for example Wang et al. 2020 Front. Pharmacol., 28 Feb. 2020).

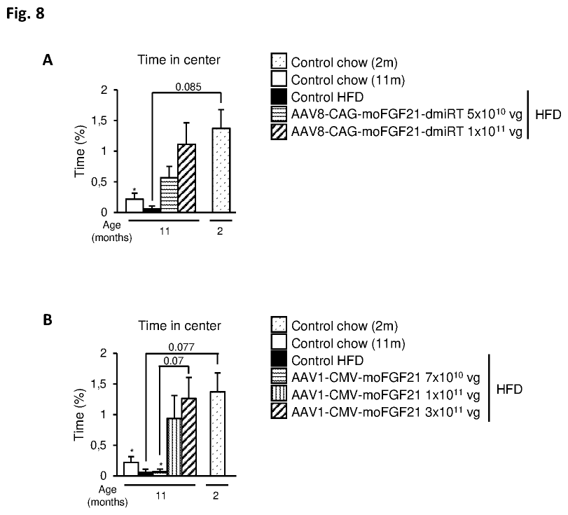

Mice displaying diet-induced obesity have been reported to mimic the anxiety-like behaviour observed in obese and diabetes patients (Asato et al, Nihon Shinkei Seishin Yakurigaku Zasshi, 32 (5-6), 251-5 (2012)). We examined the anxiety-like behaviour by means of the open field test, which is widely used to assess this parameter in mice (Zhang, L-L. et al., 2011, Neuroscience, 196, 203-14). Mice prefer to move around the periphery of an apparatus when they are placed in an open field of a novel environment. Therefore, the time spent in the central area of the open field is considered to be inversely correlated to their level of anxiety-related proneness. 16.5-month-old untreated HFD-fed mice spent less time in the central zone as compared to age-matched chow-fed controls, suggesting an enhanced level of anxiety (FIG. 8A). In marked contrast, treatment with AAV8-CAG-moFGF21-dmiRT vectors completely counteracted anxiety; in particular, the time spent in the central zone by HFD-fed mice treated with 1×10¹¹ vg of AAV8-CAG-moFGF21-dmiRT was similar to that of 2-month-old chow-fed control mice (FIG. 8A). Intramuscular administration of 1×10¹¹ and 3×10¹¹ vg of AAV1-CMV-moFGF21 vectors also mediated counteraction of HFD-associated anxiety (FIG. 8B).

All these results suggest that treatment with FGF21-encoding AAV vectors improved the behavioural deficits associated with diabetes and obesity.

### Example 3. Counteraction of Anxiety and Improvement of Neuromuscular Performance and Cognition in HFD-Fed Female Mice Treated with AAV Vectors Encoding FGF21

Next, we evaluated whether im administration of AAV1-CMV-moFGF21 vectors may mediate therapeutic benefit in obese and insulin resistance female mice. To this end, 11-week-old female C57Bl6 mice were fed a HFD for 8 weeks and subsequently treated in the quadriceps, gastrocnemius and tibialis cranialis skeletal muscle with AAV1-CMV-moFGF21 vectors at doses of 1×10¹¹ or 3×10¹¹ vg/mouse. Untreated chow and HFD-fed cohorts served as controls.

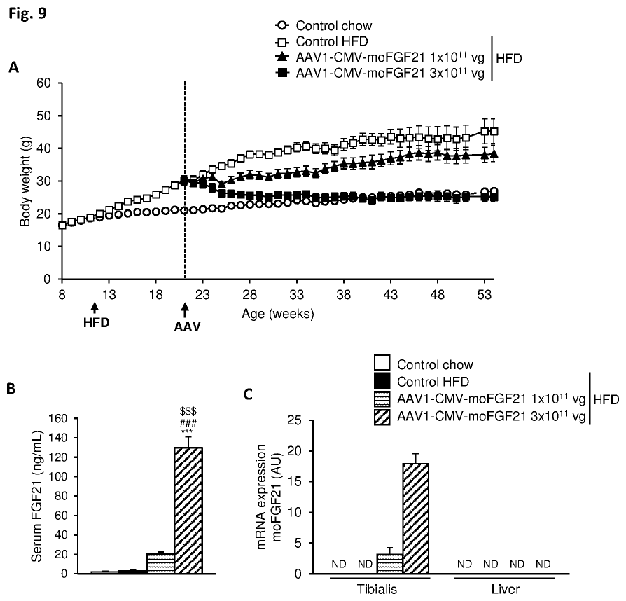

Female mice treated with 1×10¹¹ vg of AAV1-CMV-moFGF21 vectors initially lost 5% body weight and showed always a mean weight lower than that of control HFD-fed mice (FIG. 9A). Noticeably, the cohort of mice treated with 3×10¹¹ vg of AAV1-CMV-moFGF21 vectors normalized their body weight within a few weeks of AAV delivery (FIG. 9A). Indeed, the mean body weight of this group of animals became indistinguishable from that of the chow-fed, untreated cohort for the duration of the follow-up period (˜8 months) (FIG. 9A).

Similar to the observations made in HFD-fed male mice treated im with AAV1-CMV-moFGF21 vectors, genetic engineering of the skeletal muscle of female mice with the same vectors also mediated a marked increase in circulating FGF21 levels (FIG. 9B) and specific overexpression of the factor in the injected muscles (FIG. 9C).

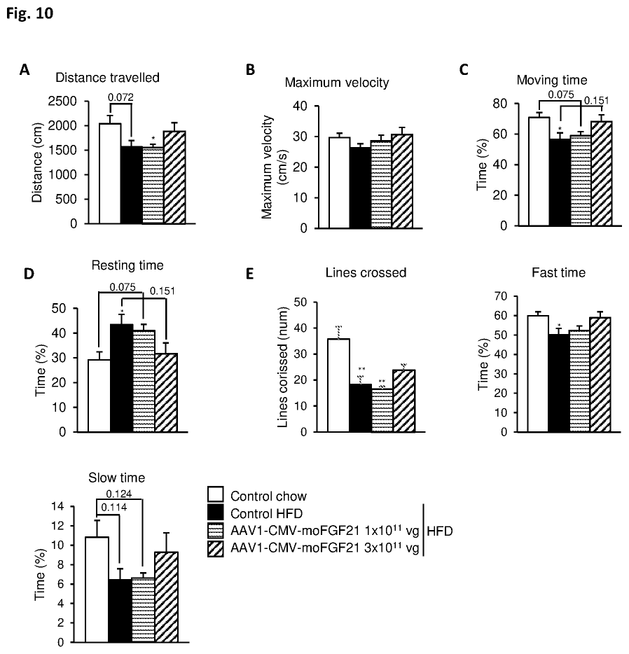

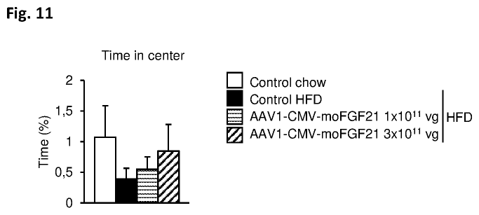

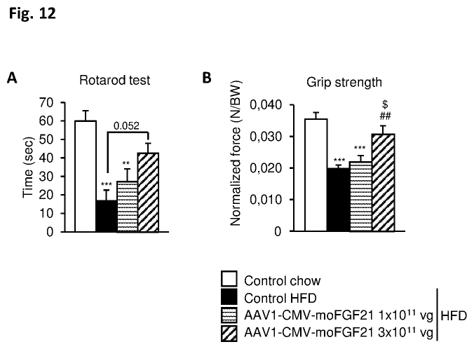

To assess neuromuscular performance, the open field, the rotarod, and the grip strength tests were performed. During the open field test, female mice fed a HFD and overexpressing FGF21 in the skeletal muscle showed increased locomotor activity (FIG. 10). Behavior that is typically characterized as depression-like behavior in the open-field test, such as reduced total distance travelled (see for example Wang et al. 2020 Front. Pharmacol., 28 Feb. 2020), was also improved. Noticeably, the open field test also revealed decreased anxiety in mice treated with AAV1-CMV-moFGF21 vectors (FIG. 11). In addition, female mice administered im with 3×10¹¹ vg of AAV1-CMV-FGF21 vectors were able to stay longer on the accelerating rotarod than untreated HFD-fed counterparts, demonstrating improvement of coordination and balance (FIG. 12A). Moreover, the former mice also displayed higher muscle strength than untreated obese mice, being grip strength of the former mice slightly reduced in comparison with that of chow-fed mice (FIG. 12B).

To test the effect of the treatment with AAV1-CMV-FGF21 vectors on cognitive performance, the novel object recognition and the Y-maze tests were performed. HFD-fed female mice treated with FGF21-encoding vectors performed markedly better than the untreated HFD-fed cohort in both tests. In the novel object recognition test, mice receiving 3×10¹¹ vg/mouse of AAV1-CMV-FGF21 vectors had a recognition index equivalent to that of the chow-fed control cohort whereas mice treated with the dose of 1×10¹¹ vg displayed better learning and memory than control lean animals (FIG. 13A). In addition, mice treated with AAV1-CMV-FGF21 vectors showed improved spatial memory in the Y-maze, irrespective of the dose (FIG. 13B). Control chow-fed and HFD-fed mice treated with 1×10¹¹ or 3×10¹¹ vg/mouse of AAV1-CMV-FGF21 vectors explored the new arm similarly and more frequently than the other arms (FIG. 13B).

All these results suggest that treatment with FGF21-encoding AAV vectors improved the neuromuscular and cognitive decline associated with diabetes and obesity.

### Example 4. Increased Locomotor Activity and Amelioration of Anxiety-Like Behaviour, Exploratory Capacity and Cognition in db/db Mice Treated with AAV Vectors Encoding FGF21

The therapeutic potential for cognitive decline of the AAV-mediated genetic engineering of the brain with FGF21 gene therapy was evaluated in db/db mice. db/db mice are a widely used genetic mouse model of obesity and diabetes, characterized by a deficit in leptin signalling. Moreover, db/db mice have also been used as a mice model of neuroinflammation and cognitive decline (Dey et al, J. Neuroimmmunol. 2014; Dinel et al Plos one 2011; Stranahan et al Nat Neurosci 2008; Zheng, Biochimica and Biophysica Acta 2017).

Two-month-old db/db male mice were administered locally intra-cerebrospinal fluid (CSF), through the cisterna magna, with 5×10¹⁰ vg/mouse of AAV1 vectors encoding a murine codon-optimized FGF21 coding sequence under the control of the CAG ubiquitous promoter (AAV1-CAG-moFGF21). As controls, non-treated db/db and non-treated db/+(lean) mice were used.

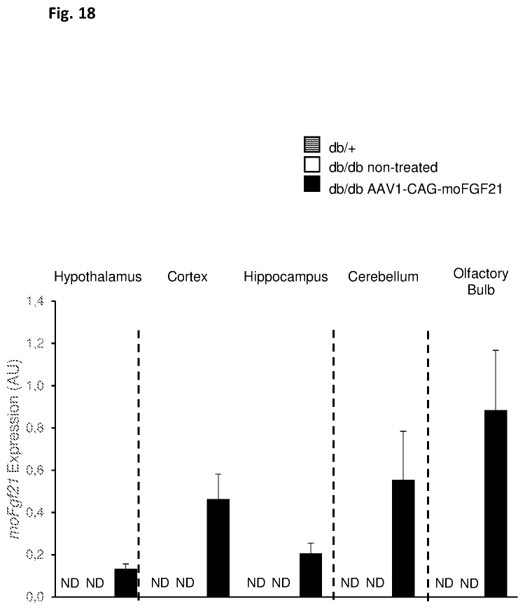

Intra-CSF administration of AAV1-CAG-moFGF21 vectors mediated widespread overexpression of FGF21 in the brain, as evidenced by the increased expression levels of the factor in different areas of the brain such as hypothalamus, cortex, hippocampus, cerebellum and olfactory bulb, 16 weeks after AAV administration (FIG. 18).

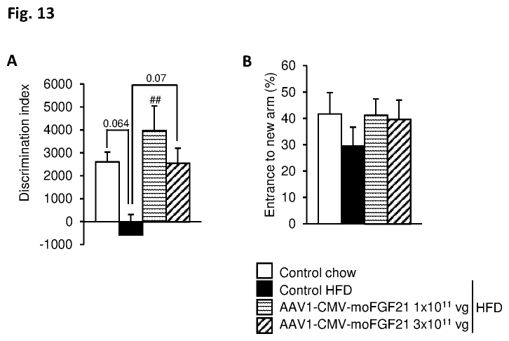

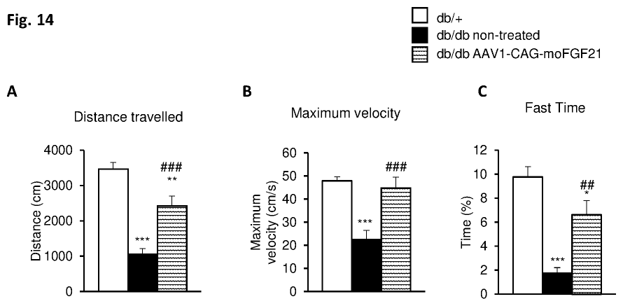

An open field test was performed at 9 weeks of age to all groups of mice. Non-treated db/db mice showed a reduction in the distance travelled, the maximum velocity and in the fast time (FIG. 14A-C). All these parameters were ameliorated in db/db mice after AAV1-CAG-moFGF21 administration (FIG. 14A-C), indicating increased locomotor activity after FGF21 gene therapy treatment. Behavior that is typically characterized as depression-like behavior in the open-field test, such as reduced total distance travelled (see for example Wang et al. 2020 Front. Pharmacol., 28 Feb. 2020), was also improved.

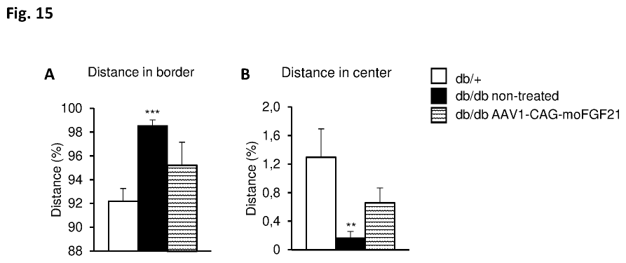

The anxiety-like behaviour was also studied in the open field, and the impairment observed in db/db non-treated mice (increased distance in the border and reduced distance in the center) (FIG. 15A-B) was ameliorated in db/db mice after AAV1-CAG-moFGF21 intra-CSF administration (FIG. 15A-B), indicating a reduction in the anxiety-like behaviour.

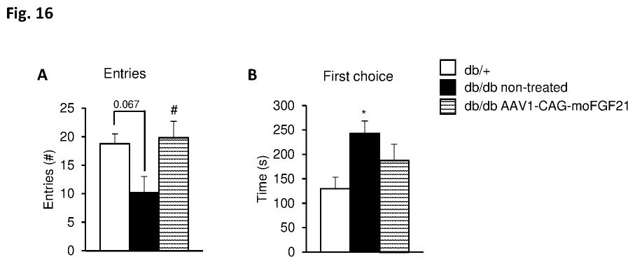

An Y-maze test was performed to all groups of mice at 10 weeks of age and showed that non-treated db/db mice had less exploratory capacity than db/+ lean mice (FIG. 16A-B), and that the exploratory capacity of db/db mice was ameliorated after intra-CSF treatment with AAV1-CAG-moFGF21 gene therapy (increased number of entries and reduced first choice latency) (FIG. 16A-B).

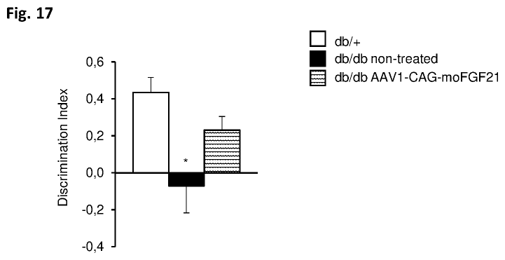

To test the effect of the intra-CSF treatment with AAV1-CAG-moFGF21 vectors on memory, the novel object recognition test was performed at 11 weeks of age. db/db mice treated with AAV1-CAG-moFGF21-encoding vectors performed markedly better than the untreated db/db cohort (FIG. 17). Moreover, the marked reduction in the discrimination index observed in non-treated db/db mice was highly ameliorated after the AAV1-CAG-moFGF21 administration (FIG. 17), indicating increased memory after the gene therapy.

### Example 5 Decreased Neuroinflammation Indicating Reduction of Depression in db/db and SAMP8 Mice Treated with AAV Vectors Encoding FGF21

We also evaluated the potential of the AAV-mediated FGF21 gene therapy for decreasing neuroinflammation.

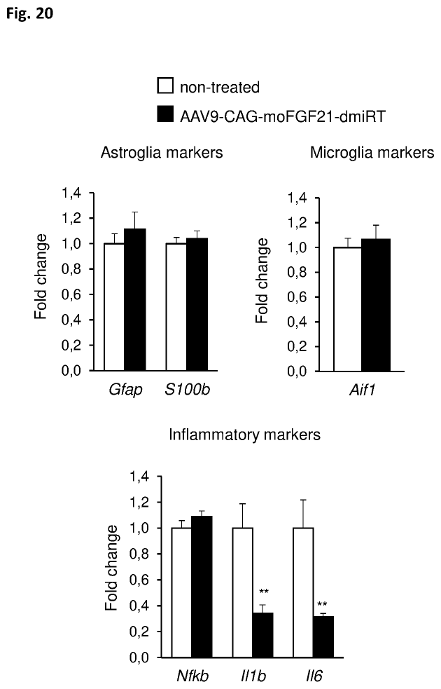

First, we used a senescence-accelerated mouse-prone 8 (SAMP8) mice, which is a widely used mouse model of senescence with age-related brain pathologies such as neuroinflammation (Takeda T., Neurochem. Res. 2009, 34(4):639-659; Griñan-Ferré C. et al. Mol. Neurobiol. 2016, 53(4):2435-2450). Inflammation in the brain was analyzed through the expression of astrocyte markers Gfap and S100b, the microglia marker Aif1 and pro-inflammatory molecules, such as Nfkb, II1b and II6. Expression of the pro-inflammatory cytokines 111b and 116 was decreased in the hypothalamus of SAMP8 mice overexpressing FGF21 in the brain (FIG. 20).

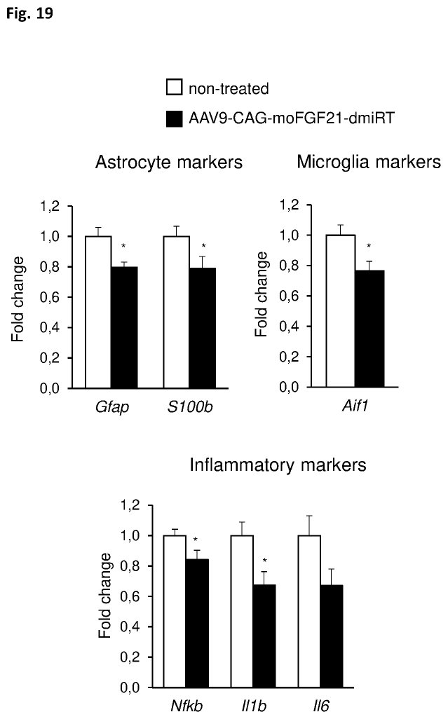

Second, we used db/db mice which are a widely used genetic mouse model of obesity and diabetes, characterized by a deficit in leptin signalling. Moreover, these mice present not only inflammation in peripheral tissues such as adipose tissue and liver but also in the brain (Dey et al, J. Neuroimmmunol. 2014). db/db mice treated intra-CSF with AAV9-CAG-moFGF21-dmiRT vectors showed decreased expression of Gfap, S100b, Aif1, Nfkb, 111b and 116 in the hypothalamus (FIG. 19).

The decrease in astrocyte markers accompanied with a decrease in the expression levels of the inflammatory cytokines indicates that after FGF21 gene therapy treatment there is a decrease in the population of deleterious astrocytes (A1 astrocytes) and also a decrease in microglia.

Many studies have supported that inflammatory processes play a central role in the aetiology of depression (Wang et al. 2020 Front. Pharmacol., 28 Feb. 2020). Together with the reduced depression-like behaviour observed in examples 2, 3 and 4, this indicates that the FGF21 gene therapy has an anti-depressant effect.

### Example 6. Intramuscular Administration of AAV1-CMV-moFGF21 Vectors in SAMP8 Mice

To further evaluate the therapeutic potential of the AAV-mediated genetic engineering of the skeletal muscle with FGF21 on cognitive decline, SAMP8 mice are used. The SAMP8 mouse model presents cognitive decline by the age of 8-12 months (Miyamoto, M., Physiol Behay. 1986; 38(3):399-406; Markowska, A L., Physiol Behay. 1998; 64(1):15-26).

SAMP8 mice are administered im with 3×10¹¹ vg/mouse of AAV1-CMV-moFGF21 vectors. As control, non-treated SAMP8 and SAMR1 animals are used. Several behavioural and neuromuscular tests such as Y-Maze, Open-Field, novel object recognition test, rotarod, hang wire test, grip strength test and Morris Water Maze are performed in these mice. At sacrifice, serum and tissue samples are taken for analysis. Analysis of these samples include studies on neurogenesis (expression of neuronal markers such as Sox2, NeuN, and Dcx), neuroinflammation (expression of GFAP, Iba1 and several cytokine levels), studies on synaptic degeneration (protein levels of synaptophysin and spine density).

### Example 7. Intramuscular Administration of AAV1-CMV-moFGF21 Vectors in an Alzheimer's Disease Mouse Model

To evaluate the therapeutic potential of the AAV-mediated genetic engineering of the skeletal muscle with FGF21 on Alzheimer's disease, the 3×Tg-AD (B6; 129Tg(APPSwe,tauP301L)1Lfa Psen1^{tm1Mpm}) mouse model is used. The 3×Tg-AD is a widely used mouse model of Alzheimer's disease, homozygous for all three mutant alleles, homozygous for the Psen1 mutation and homozygous for the co-injected APPSwe and tauP301 L transgenes (Belfiore, R., Aging Cell. 2019, 18(1):e12873)

3×Tg-AD mice are administered im with 3×10¹¹ vg/mouse of AAV1-CMV-moFGF21 vectors. As control, non-treated 3×Tg-AD animals are used. Several behavioural and neuromuscular tests such as Y-Maze, Open-Field, novel object recognition test, rotarod, hang wire test, grip strength test and Morris Water Maze are performed in these mice. At sacrifice, serum and tissue samples are taken for analysis. Analysis of these samples include studies on neurogenesis (expression of neuronal markers such as Sox2, NeuN, and Dcx), neuroinflammation (expression of GFAP, Iba1 and several cytokine levels), levels of amyloid-beta (soluble amyloid and plaques), studies on synaptic degeneration (protein levels of synaptophysin and spine density), levels of tau phosphorylation.

### Example 8. Improved Neuromuscular Performance and Cognition in SAMP8 Mice Treated Intramuscularly with AAV1-CMV-moFGF21 Vectors

Eight-week-old male SAMP8 mice were administered im with 3×10¹¹ vg/mouse of AAV1-CMV-moFGF21 vectors. As control, non-treated SAMP8 and SAMR1 animals were used.

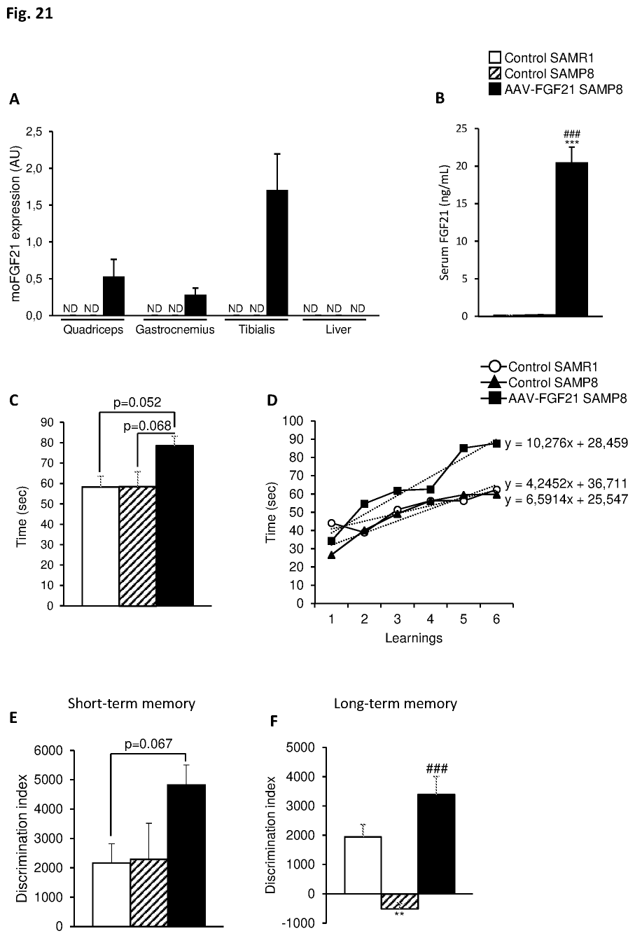

AAV1-CMV-moFGF21 treated SAMP8 mice showed specific overexpression of codon-optimized FGF21 in the three injected muscles and increased FGF21 circulating levels (FIG. 21A-B).

To test the effect of the treatment with AAV1-CMV-moFGF21 vectors on neuromuscular performance, the rotarod test was performed. SAMP8 mice administered im with 3×10¹¹ vg of AAV1-CMV-moFGF21 vectors were able to stay longer on the accelerating rotarod than untreated SAMP8 and SAMR1, demonstrating improvement of coordination and balance (FIG. 21C). Motor learning ability was also assessed by examining performance improvement during subsequent trials. Noticeably, AAV1-FGF21 treated SAMP8 mice outperformed untreated SAMP8 and SAMR1 counterparts (FIG. 21D). Moreover, the novel object recognition test further confirmed prevention of cognitive decline in SAMP8 mice treated with FGF21-encoding vectors. By 32 weeks of age, treated SAMP8 mice showed marked improvement of short- and long-term memory in comparison with both untreated SAMP8 mice and control SAMR1 mice (FIG. 21E-F).

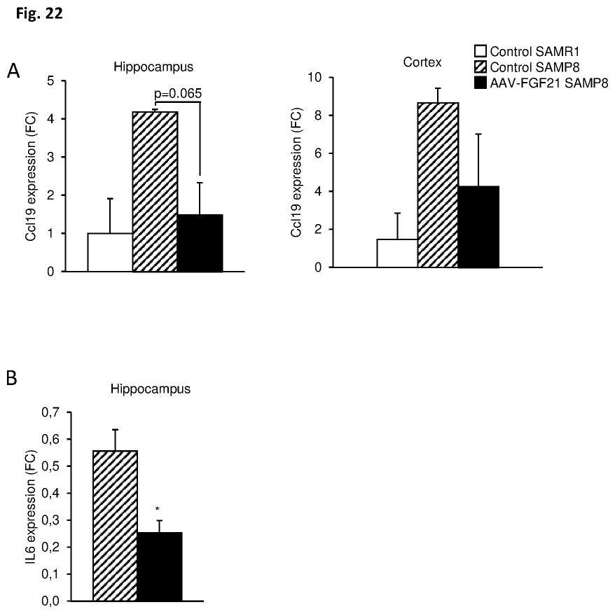

Inflammation in the brain was analyzed through the expression of chemokine (C—C motif) ligand 19 (Ccl19) and II6. Cccl19 has been postulated to play a primary role on the neuropathological phenotype of SAMP8 (Carter T A. Genome Biol. 2005; 6(6):R48). SAMP8 showed markedly increased Ccl19 expression levels in cortex and hippocampus in comparison with SAMR1 mice (FIG. 22A). Treatment of SAMP8 mice with AAV1-CMV-moFGF21 vectors normalized Ccl19 expression levels in such brain areas (FIG. 22A). Moreover, AAV1-FGF21-treated SAMP8 showed decreased 116 expression in hippocampus (FIG. 22B).

All these results suggest that treatment with AAV1-FGF21 enhanced neuromuscular performance, motor learning and memory, and decreased brain inflammation in SAMP8 mice.

### Example 9. Improved Memory in an Alzheimer's Disease Mouse Model Treated Intramuscularly with AAV1-CMV-moFGF21 Vectors

Eight-week-old male 3×Tg-AD mice were administered im with 3×10¹¹ vg/mouse of AAV1-CMV-moFGF21 vectors. As control, non-treated 3×TG-AD and B6129SF2/J animals were used.

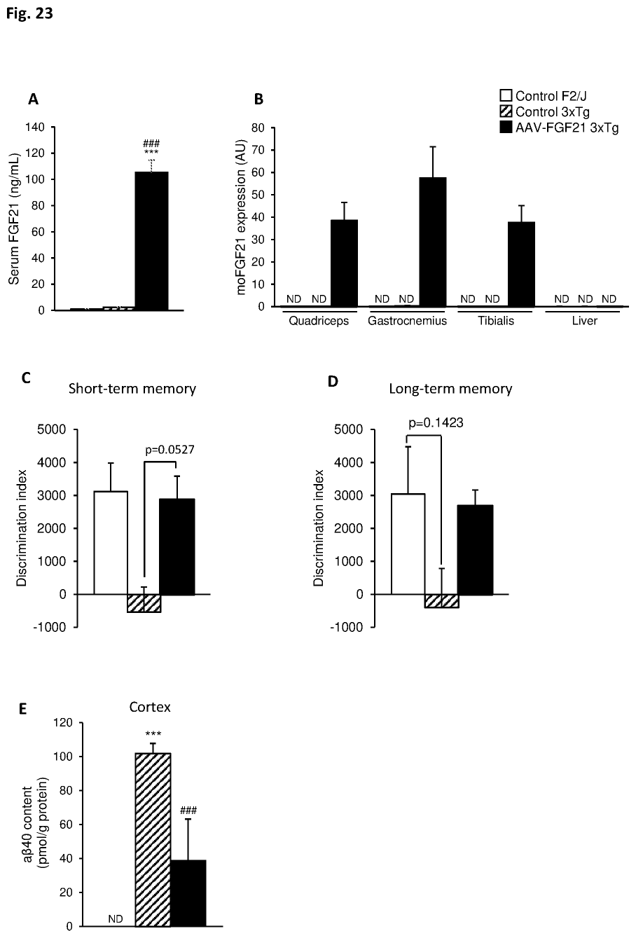

Similar to the observations made in SAMP8 mice treated im with AAV1-CMV-moFGF21 vectors, genetic engineering of the skeletal muscle of 3×Tg-AD mice with the same vectors also mediated a marked increase in circulating FGF21 levels (FIG. 23A) and specific overexpression of the factor in the injected muscles (FIG. 23B).

Accumulation of amyloid plaques (primary made of amyloid-β (Aβ)) in brain and memory loss are key hallmarks of Alzheimer's disease (Belfiore R. et al. Aging Cell. 2019; 18(1):e12873). Treatment of 3×Tg-AD mice with AAV1-CMV-moFGF21 vectors precluded cognitive decline as demonstrated by the markedly improved short- and long-term memory in treated 3×Tg-AD mice in comparison with untreated 3×Tg-AD mice (FIG. 23C-D). Of note, discrimination indexes of AAV1-FGF21-treated 3×Tg-AD mice were similar to those of control B6129SF2/J animals (FIG. 23C-D). Moreover, 3×Tg-AD mice treated im with AAV1-CMV-moFGF21 vectors showed markedly reduced insoluble Aβ₄₀ levels in cortex in comparison with untreated 3×Tg-AD mice (FIG. 23E).

### Example 10. Improved Neuromuscular Performance and Cognition in Old Mice Treated im with Different Doses of AAV1-CMV-moFGF21 Vectors

Thirteen-month-old male C57B16 mice were administered intramuscularly with 1×10¹¹ or 3×10¹¹ vg of AAV1-CMV-moFGF21 vectors. Untreated age-matched control animals served as controls.

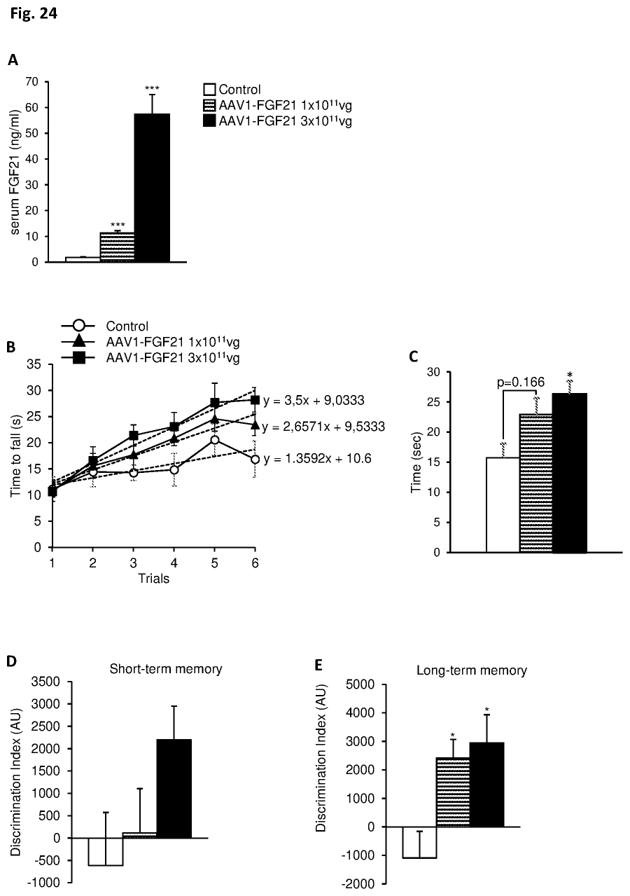

AAV1-CMV-moFGF21-treated mice showed secretion of FGF21 into the bloodstream in a dose-dependent manner (FIG. 24A). Old mice treated with AAV1-CMV-moFGF21 vectors showed improved coordination, balance and motor learning, irrespective of dose (FIG. 24B-C). Moreover, treatment of old mice with 1×10¹¹ or 3×10¹¹ vg of AAV1-CMV-moFGF21 vectors markedly improved short- and long-term memory (FIG. 24D-E).

### Example 11. Molecular Mechanisms and Brain Areas Involved in Preclusion of Neurodegeneration and Cognitive Decline in Old Mice Treated im with AAV1-CMV-moFGF21

As previously mentioned, whole brain transcriptomic analysis suggested that improvement of mitochondrial function and diminution of oxidative stress mediated inhibition of neurodegeneration and cognitive decline in old mice treated im AAV1-CMV-moFGF21 vectors (Table 1). Next, we characterized the specifically affected brain areas as well as decipher additional molecular mechanisms involved in the improvement of cognitive performance in AAV1-FGF21 treated mice.

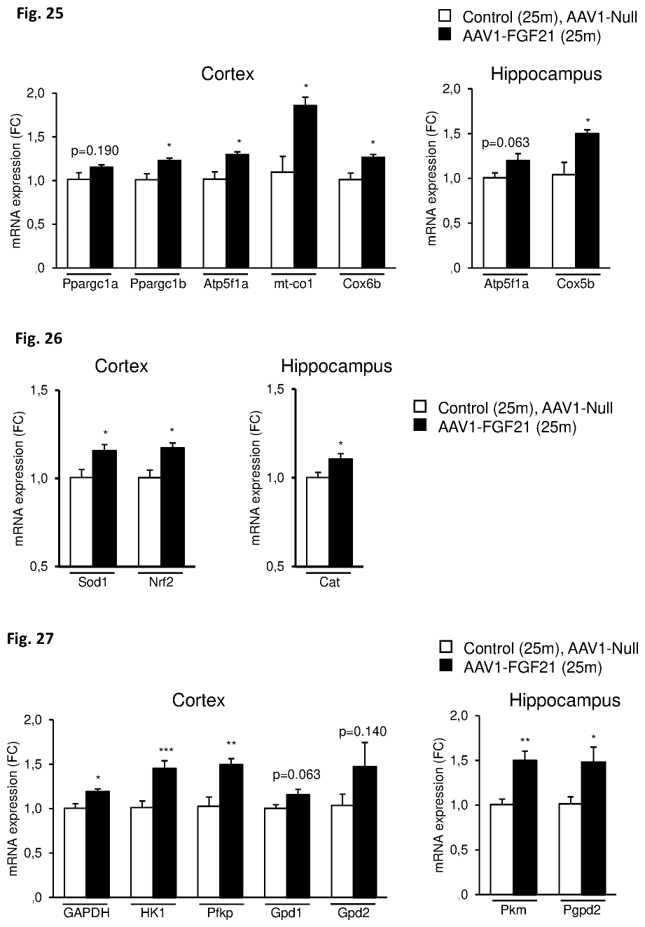

Measurement of several oxidative phosphorylation (OXPHOS) and antioxidant markers by qPCR further corroborated GSEA findings (FIGS. 25 and 26). Moreover, qPCR analysis revealed enhancement of OXPHOS predominantly in cortex and to a lesser extent in hippocampus (FIG. 25), both key brain areas involved in cognitive function. In detail, old animals treated im with 3×10¹¹ vg of AAV1-CMV-moFGF21 vectors showed increased expression of peroxisome proliferator-activated receptor gamma coactivator 1 alpha and beta (Ppargc1a and Ppargc1b, respectively) in cortex and of their transcriptional targets ATP Synthase F1 Subunit alpha (Atp5f1a), cytochrome c oxidase 1 (mt-co1) and cytochrome c oxidase subunit 6 (Cox6) in cortex and of Atp5f1a and cytochrome c oxidase subunit 5a (Cox5a) in hippocampus in comparison with age-matched counterparts (FIG. 25) (Sahin, E. et al. Nature. 2011; 470(7334):359-65). Similarly, increased expression of the transcription factor NF-E2-related factor 2 (Nrf2), which coordinates activation of antioxidant gene expression (Jaiswal A K, 2004. Free Radic Biol Med 36:1199-1207; Lee J M, 2004. J Biochem Mol Biol 37:139-143) and of key genes encoding reactive oxygen species detoxifying enzymes such as superoxide dismutase 1 (Sod1) and catalase (Cat), which are also Ppargc1a targets (Sahin, E. et al. Nature. 2011; 470(7334):359-65), were documented in cortex and hippocampus of AAV1-CMV-moFGF21-treated old mice (FIG. 26).

The brain is an energy-demanding organ and relies heavily on efficient ATP production via glycolysis, the TCA cycle and oxidative phosphorylation (Butterfield D A. Nat Rev Neurosci 2019 March; 20(3):148-160). Given that glycolysis is in charge of metabolization of glucose for OXPHOS, expression levels of key glycolysis-related genes were determined. Old mice treated with AAV1-CMV-moFGF21 vectors showed increased expression of glycerldehyde-3-phosphate dehydrogenase (GAPDH), hexokinase 1 (Hk1), platelet isoform of phosphofructokinase (Pfkp) and glycerol-3-Phosphate Dehydrogenase 1 and 2 (Gpd1 and Gpd2, respectively) in cortex and of pyruvate kinase M (Pkm) and Gpd2 in hippocampus, suggesting enhanced glycolysis in these brain areas (FIG. 27).

All these results suggest that treatment of old mice with AAV1-CMV-moFGF21 precluded age-associated decreased glucose metabolism and mitochondrial dysfunction, which would ensure efficient ATP production for neuronal function.

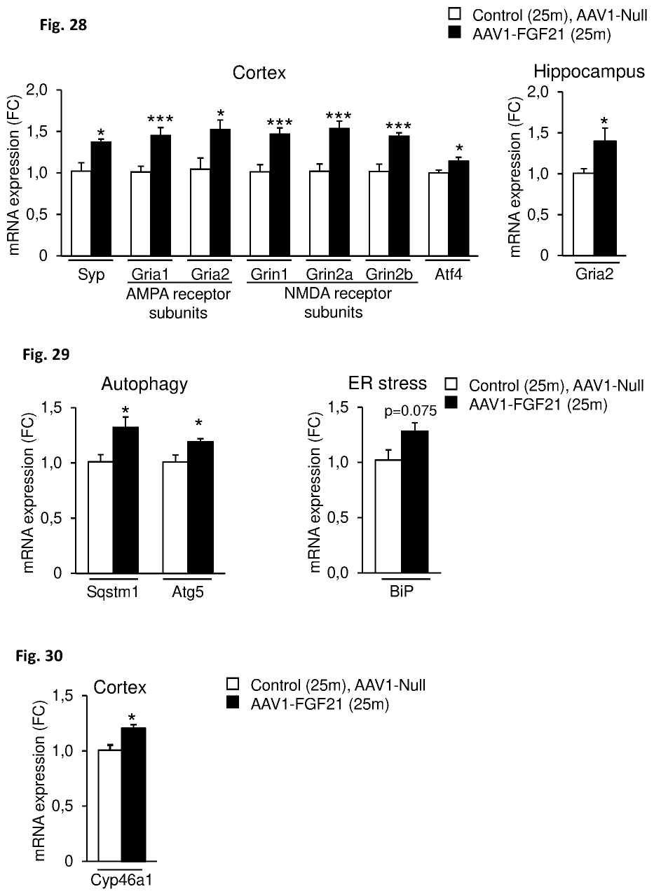

It is worth to mention that mitochondrial perturbations and reduced ATP production have been reported to contribute to synaptic dysfunction and degeneration, which correlate strongly with cognitive deficits and memory loss (Butterfield D A. Nat Rev Neurosci 2019 March; 20(3):148-160; Cai Q. J Alzheimers Dis. 2017; 57(4):1087-1103). Noticeably, by 25 months of age, old animals treated with AAV1-CMV-moFGF21 showed robust increased expression of key synaptic proteins. (FIG. 28). Specifically, expression levels of synaptophysin (Syp), GluR1 and GluR2 subunits of the alpha-amino-3-hydroxy-5-methyl-4-isoxazole proprionic acid (AMPA)-type (Gria1 and Gria2, respectively) and NR1, N2A and N2B subunits of the N-methyl-d-aspartate (NMDA)-type (Grin1, Grin2a, Grin2b) ionotropic glutamate receptors were increased in cortex (FIG. 28). Gria2 was also increased in hippocampus (FIG. 28). Moreover, increased expression levels of activating transcription factor 4 (Atf4), a key transcription factor involved in a wide range of activities, including regulation of synaptic plasticity and memory (III-Raga g. Hippocampus 2013; 23:431-436; Liu J. Front Cell Neurosci. 2014; 8:177) were detected in cortex (FIG. 28). The enhanced expression of key synaptic proteins would likely improve synaptic plasticity and, as a result, cortex and hippocampal function.

Brain autophagic capacity has been reported to decrease with age and to cause neurodegeneration (Lipinski M M. Proc Natl Acad Sci USA 2010; 107:14164-9; Hara T. Nature 2006; 441:885-9; Komatsu M. Nature 2006; 441:880-4). Old mice treated im with AAV1-CMV-moFGF21 vectors showed increased expression of the autophagy markers p62 (encoded by the Sqstm1 gene) and autophagy related 5 (Atg5) in cortex (FIG. 29). Similarly, the endoplasmic reticulum (ER) also plays an essential role in cellular homeostasis. Induction of the anti-apoptotic chaperone BiP (also known as GRP78) may represent a major cellular protective mechanism for cells to survive ER stress (A. S. Lee, Trends Biochem. Sci. 26 (2001) 504-510). In this regard, increased expression of BiP was observed in cortex of AAV1-FGF21-treated old mice (FIG. 29). Atf4, whose expression was also induced in cortex of old mice treated im with AAV1-CMV-moFGF21 (FIG. 28), has been reported to upregulate BiP expression (Luo S. J Biol Chem. 2003; 278(39):37375-85).

Finally, a strong association between abnormalities in brain cholesterol homeostasis (especially high concentrations in neurons) and several neurodegenerative disorders, including Alzheimer's disease, Parkinson's disease and Huntington's disease, has been observed (Vance J E. Dis Model Mech 2012; 5:746-55). Cholesterol 24-hydroxylase, encoded by Cyp46a1, controls cholesterol efflux from the brain and thereby plays a major role in regulating brain cholesterol homeostasis. Moreover, increasing evidence suggest that Cyp46a1 has a role in the pathogenesis and progression of neurodegenerative disorders, and that increasing its levels in the brain is neuroprotective (Kacher R. Brain. 2019; 142(8):2432-2450; Djelti F. Brain 2015; 138(Pt 8):2383-98). In agreement, treatment with AAV1-CMV-moFGF21 vectors increased the expression of Cyp46a1 in cortex of old mice (FIG. 30).

Altogether, these results indicate that FGF21 gene therapy inhibited neurodegeneration and cognitive decline by improvement of mitochondrial function, increase of glucose metabolism and autophagia, diminution of oxidative and ER stress, and amelioration of cholesterol homeostasis and synaptic function in cortex and hippocampus of old mice.

### Example 12. Counteraction of Anxiety and Improvement of Neuromuscular Performance and Cognition in HFD-Fed Male Mice Treated Intra-CSF with AAV Vectors Encoding FGF21

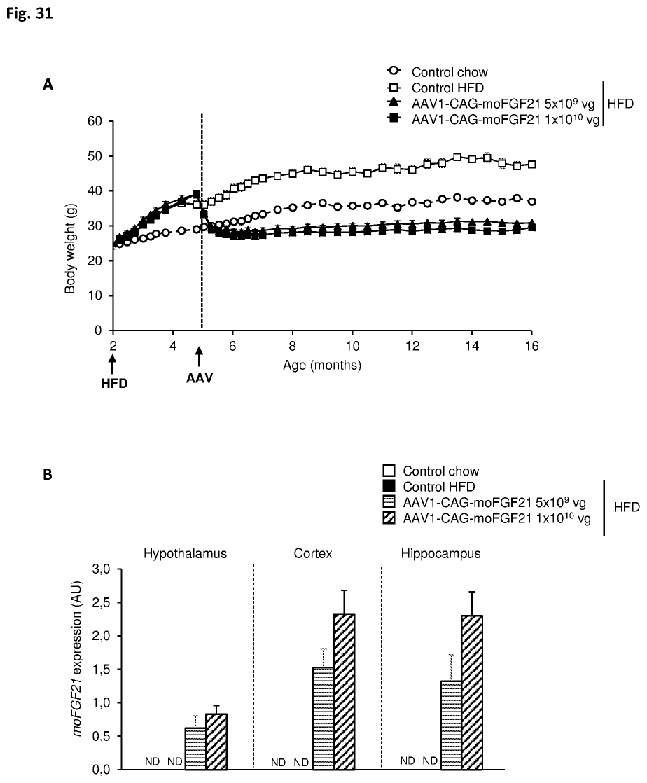

We next evaluated whether intra-CSF administration of AAV1-CAG-moFGF21 vectors may mediate therapeutic benefit in obese and insulin resistance male mice. To this end, 8-week-old male C57Bl6 mice were fed a HFD for 3 months. During these first 3 months of follow-up the body weight of chow-fed animals increased by 32% while animals fed a HFD became obese (84% body weight gain). Obese animals were then administered intra-CSF with 5×10⁹ or 1×10¹⁰ vg/mouse of AAV1-CAG-moFGF21 vectors. Untreated chow and HFD-fed cohorts served as controls. Initially, HFD-fed mice treated with AAV1 vectors lost body weight, reaching similar levels than those of age-matched chow-diet fed mice (FIG. 31A). Two months after AAV1 treatment, the body weight of HFD-fed mice was stabilized and remained to similar levels during the follow-up of the experiment (˜11 months) (FIG. 31A).

Similar to the observations made in db/db male mice treated intra-CSF with AAV1-CAG-moFGF21 vectors, genetic engineering of the brain of HFD-fed mice with the same vectors also mediated a specific overexpression of the factor in different brain areas (FIG. 31B).

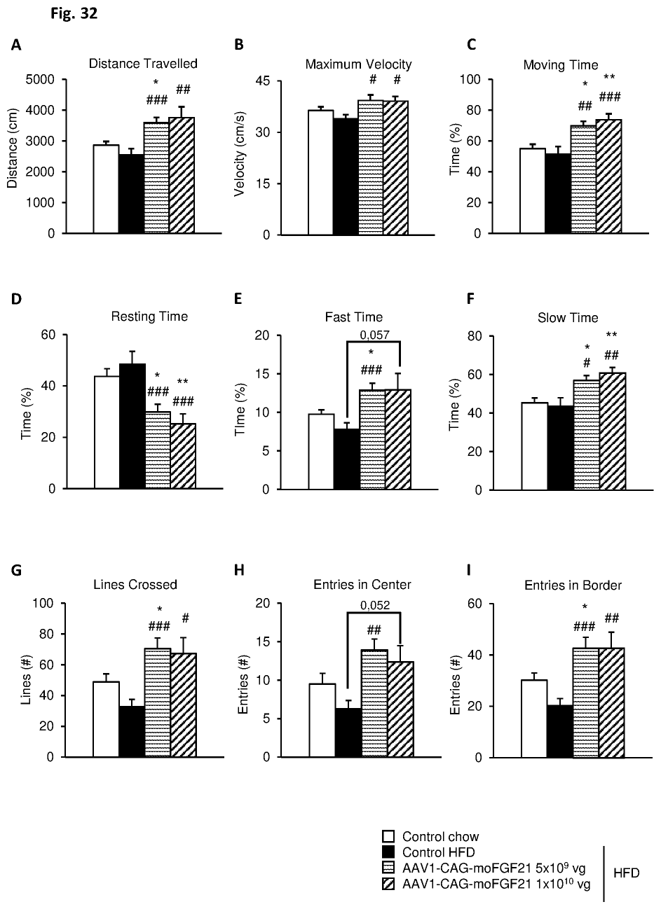

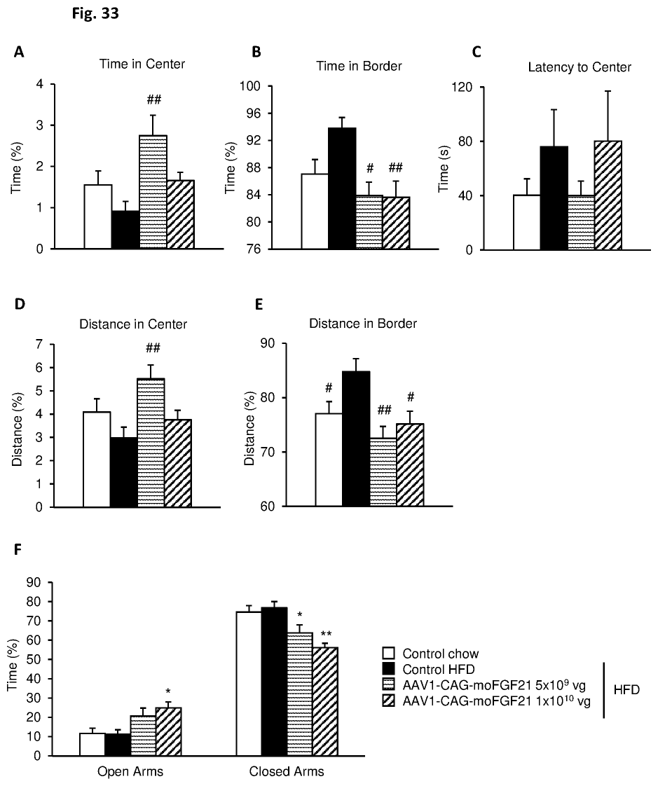

To assess neuromuscular performance, at the end of the follow-up period, the open field test was performed. During the open field test, HFD-fed mice administered intra-CSF with the two doses of the AAV1 vectors showed increased locomotor activity (FIG. 32). The increase observed in the total distance travelled also indicated an improvement in the depression-like behavior in AAV-treated mice. As a measure of anxiety, the distance and time spent in the center and in the border of the open field was measured and data showed that AAV1-CAG-moFGF21-treated mice spent more time in the center and less time in the border than control mice fed a HFD (FIG. 33A-E), indicating less anxiety-like behaviour than HFD control mice. These results were corroborated with the Elevated Plus Maze test, where FGF21-treated mice spent more time in the open arms and less time in the closed arms than HFD-fed control mice (FIG. 33F).

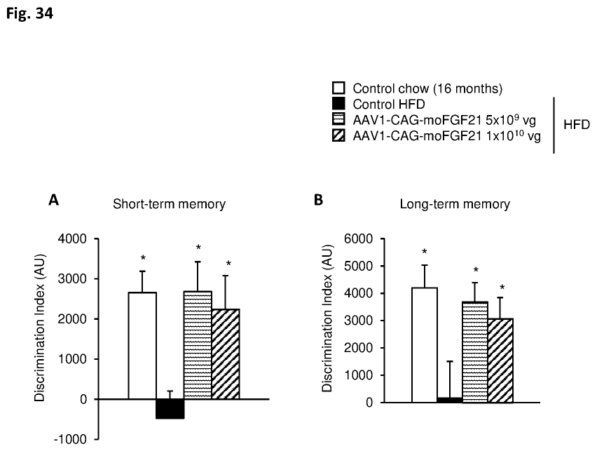

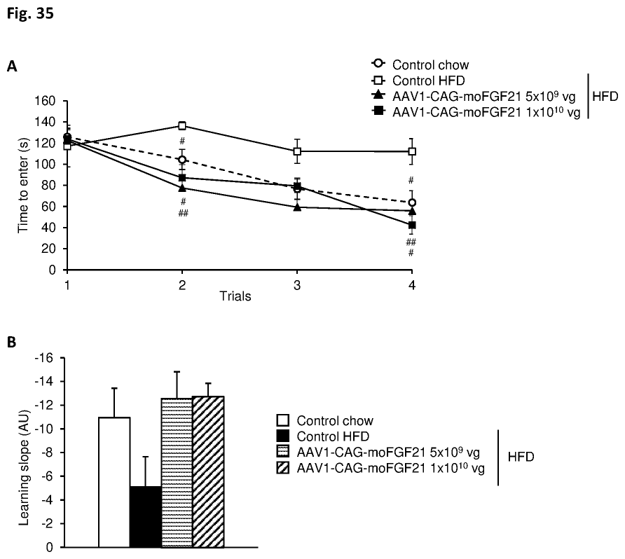

To test the effect of the intra-CSF treatment with AAV1-CAG-FGF21 vectors on cognitive performance, the novel object recognition and the Barnes maze tests were performed. In the novel object recognition test, mice receiving both doses of AAV1-CAG-FGF21 vectors had a recognition index equivalent to that of the chow-fed control cohort, both at the short and long-term memory trial (FIG. 34), whereas HFD-fed control mice showed impaired recognition index, indicating memory impairment (FIG. 34). The learning capacity of AAV1 intra-CSF treated mice was measured in the Barnes maze. The reduction observed in the time to enter the hole (FIG. 35A) and in the learning slope (FIG. 35B) of HFD-fed mice treated with the two doses of AAV1 vectors indicated that AAV-1 treated mice had increased learning capacity than control HFD-fed mice, reaching to similar levels than those of control mice fed a chow diet.

All these results suggest that intra-CSF treatment with FGF21-encoding AAV vectors improved the neuromuscular and cognitive decline associated with diabetes and obesity.

### Example 13. Improved Neuromuscular Performance and Cognition in Old Mice Treated with AAV Vectors Encoding FGF21

To evaluate whether intra-CSF gene therapy with FGF21 may exert therapeutic benefit in old animals, 13-month-old male C57Bl6 mice were administered intra-CSF with 5×10⁹ and 1×10¹⁰ vg/mouse of AAV1-CAG-moFGF21 vectors. Untreated cohorts served as controls. All experimental groups were fed with a chow diet during all the experiment.

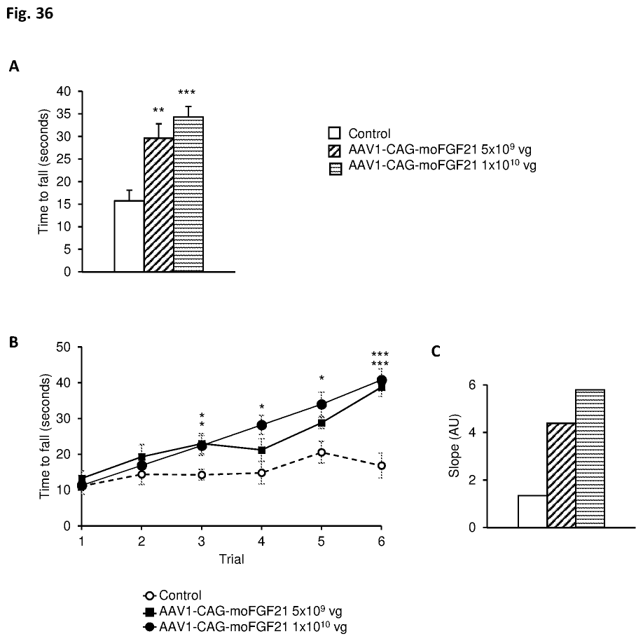

To assess neuromuscular performance, the rotarod test was performed to all groups at 23 months of age. Old mice treated intra-CSF with all doses of AAV1-CAG-moFGF21 were able to stay longer on the accelerating rotarod than untreated old counterparts (FIG. 36A), demonstrating improvement of coordination and balance. Moreover, during the different trials, there was a trial-dependent improvement in the time to fall the rotarod in AAV1 treated mice (FIG. 36B-C), indicating enhanced learning in old-treated mice.

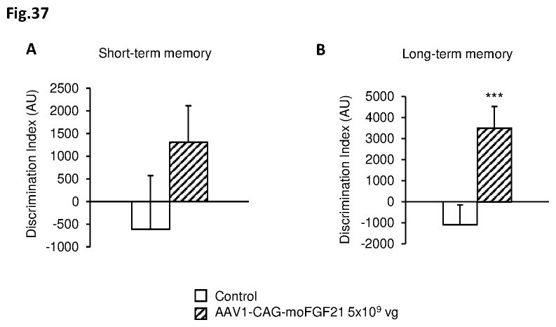

Moreover, by 24-25 months of age, mice treated with 5×10⁹ vg/mouse of FGF21-encoding vectors performed markedly better in the novel object recognition test, both at the short- and long-term trials (FIG. 37A-B), than the age-matched cohort untreated mice, suggesting that treatment with AAV1-CAG-moFGF21 vectors improved neuromuscular performance and enhanced learning and short and long-term memory in old mice.

Sequences

| SEQ ID NO: | Description of the sequence |
|---|---|
| 1 | Amino acid sequence of homo sapiens FGF21 |
| 2 | Amino acid sequence of mus musculus FGF21 |
| 3 | Amino acid sequence of canis lupus familiaris FGF21 |
| 4 | Nucleotide sequence of homo sapiens FGF21 |
| 5 | Codon optimized nucleotide sequence of homo sapiens FGF21-variant 1 |
| 6 | Codon optimized nucleotide sequence of homo sapiens FGF21-variant 2 |
| 7 | Codon optimized nucleotide sequence of homo sapiens FGF21-variant 3 |
| 8 | Nucleotide sequence of mus musculus FGF21 |
| 9 | Codon optimized nucleotide sequence of mus musculus FGF21 |
| 10 | Nucleotide sequence of canis lupus familiaris FGF21 |
| 11 | Codon optimized nucleotide sequence of canis lupus familiaris FGF21 |
| 12 | Nucleotide sequence encoding miRT-122a |
| 13 | Nucleotide sequence encoding miRT-1 |
| 14 | Nucleotide sequence encoding miRT-152 |
| 15 | Nucleotide sequence encoding miRT-199a-5p |
| 16 | Nucleotide sequence encoding miRT-199a-3p |
| 17 | Nucleotide sequence encoding miRT-215 |
| 18 | Nucleotide sequence encoding miRT-192 |
| 19 | Nucleotide sequence encoding miRT-148a |
| 20 | Nucleotide sequence encoding miRT-194 |
| 21 | Nucleotide sequence encoding miRT-133a |
| 22 | Nucleotide sequence encoding miRT-206 |
| 23 | Nucleotide sequence encoding miRT-208-5p |
| 24 | Nucleotide sequence encoding miRT-208a-3p |
| 25 | Nucleotide sequence encoding miRT-499-5p |
| 26 | Nucleotide sequence of chimeric intron composed of introns from human β-globin |
| | and immunoglobulin heavy chain genes |
| 27 | Nucleotide sequence of CAG promoter |
| 28 | Nucleotide sequence of CMV promoter |
| 29 | Nucleotide sequence of CMV enhancer |
| 30 | Truncated AAV2 5′ ITR |
| 31 | Truncated AAV2 3′ ITR |
| 32 | SV40 polyadenylation signal |
| 33 | Rabbit β-globin polyadenylation signal |
| 34 | CMV promoter and CMV enhancer sequence |
| 35 | pAAV-CAG-moFGF21-dmiRT |
| 36 | mini-CMV promoter |
| 37 | EF1α promoter |
| 38 | RSV promoter |
| 39 | Synapsin 1 promoter |
| 40 | Calcium/calmodulin-dependent protein kinase II (CaMKII) promoter |
| 41 | Glial fibrillary acidic protein (GFAP) promoter |
| 42 | Nestin promoter |
| 43 | Homeobox Protein 9 (HB9) promoter |
| 44 | Tyrosine hydroxylase (TH) promoter |
| 45 | Myelin basic protein (MBP) promoter |
| 46 | pAAV-CAG-moFGF21 |
| 47-62 and | RT-qPCR primers |
| 69-122 | |
| 63 | pAAV-CMV-moFGF21 |
| 64 | Nucleotide sequence of hAAT promoter |
| 65 | Hepatocyte control region (HCR) enhancer from apolipoprotein E |
| 66 | mini/aP2 promoter |
| 67 | mini/UCP1 promoter |
| 68 | C5-12 promoter |

Amino acid sequence of homo sapiens FGF21 (SEQ ID NO: 1)
MDSDETGFEHSGLWVSVLAGLLLGACQAHPIPDSSPLLQFGGQVRQRYLYTDDAQQTEAHLEIR
EDGTVGGAADQSPESLLQLKALKPGVIQILGVKTSRFLCQRPDGALYGSLHFDPEACSFRELLLE
DGYNVYQSEAHGLPLHLPGNKSPHRDPAPRGPARFLPLPGLPPALPEPPGILAPQPPDVGSSDP
LSMVGPSQGRSPSYAS
Nucleotide sequence of homo sapiens FGF21 (SEQ ID NO: 4)
ATGGACTCGGACGAGACCGGGTTCGAGCACTCAGGACTGTGGGTTTCTGTGCTGGCTGGTC
TTCTGCTGGGAGCCTGCCAGGCACACCCCATCCCTGACTCCAGTCCTCTCCTGCAATTCGG
GGGCCAAGTCCGGCAGCGGTACCTCTACACAGATGATGCCCAGCAGACAGAAGCCCACCTG
GAGATCAGGGAGGATGGGACGGTGGGGGGCGCTGCTGACCAGAGCCCCGAAAGTCTCCTG
CAGCTGAAAGCCTTGAAGCCGGGAGTTATTCAAATCTTGGGAGTCAAGACATCCAGGTTCCT
GTGCCAGCGGCCAGATGGGGCCCTGTATGGATCGCTCCACTTTGACCCTGAGGCCTGCAGC
TTCCGGGAGCTGCTTCTTGAGGACGGATACAATGTTTACCAGTCCGAAGCCCACGGCCTCC
CGCTGCACCTGCCAGGGAACAAGTCCCCACACCGGGACCCTGCACCCCGAGGACCAGCTC
GCTTCCTGCCACTACCAGGCCTGCCCCCCGCACTCCCGGAGCCACCCGGAATCCTGGCCC
CCCAGCCCCCCGATGTGGGCTCCTCGGACCCTCTGAGCATGGTGGGACCTTCCCAGGGCC
GAAGCCCCAGCTACGCTTCCTGA
Codon optimized nucleotide sequence of homo sapiens FGF21-variant 1 (SEQ ID NO: 5)
ATGGATTCTGATGAGACAGGCTTCGAGCACAGCGGCCTGTGGGTTTCAGTTCTGGCTGGAC
TGCTGCTGGGAGCCTGTCAGGCACACCCTATTCCAGATAGCAGCCCTCTGCTGCAGTTCGG
CGGACAAGTGCGGCAGAGATACCTGTACACCGACGACGCCCAGCAGACAGAAGCCCACCT
GGAAATCAGAGAGGATGGCACAGTTGGCGGAGCCGCCGATCAGTCTCCTGAATCTCTGCTC
CAGCTGAAGGCCCTGAAGCCTGGCGTGATCCAGATCCTGGGCGTGAAAACCAGCCGGTTCC
TGTGCCAAAGACCTGACGGCGCCCTGTATGGCAGCCTGCACTTTGATCCTGAGGCCTGCAG
CTTCAGAGAGCTGCTGCTTGAGGACGGCTACAACGTGTACCAGTCTGAGGCCCATGGCCTG
CCTCTGCATCTGCCTGGAAACAAGAGCCCTCACAGAGATCCCGCTCCTAGAGGCCCTGCCA
GATTTCTGCCTCTTCCTGGATTGCCTCCTGCTCTGCCAGAGCCTCCTGGAATTCTGGCTCCT
CAGCCTCCTGATGTGGGCAGCTCTGATCCTCTGAGCATGGTCGGACCTAGCCAGGGCAGAT
CTCCTAGCTACGCCTCTTGA
Codon optimized nucleotide sequence of homo sapiens FGF21-variant 2 (SEQ ID NO: 6)
ATGGACAGCGATGAAACCGGGTTCGAGCACAGCGGTCTGTGGGTGTCCGTGCTGGCCGGA
CTGCTCCTGGGAGCCTGTCAGGCGCACCCCATCCCTGACTCCTCGCCGCTGCTGCAATTCG
GCGGACAAGTCCGCCAGAGATACCTGTACACCGACGACGCCCAGCAGACCGAAGCCCACC
TGGAAATTCGGGAGGACGGGACTGTGGGAGGCGCTGCAGATCAGTCACCCGAGTCCCTCC
TCCAACTGAAGGCCTTGAAGCCCGGCGTGATTCAGATCCTGGGCGTGAAAACTTCCCGCTT
CCTTTGCCAACGGCCGGATGGAGCTCTGTACGGATCCCTGCACTTCGACCCCGAAGCCTGC
TCATTCCGCGAGCTGCTCCTTGAGGACGGCTATAACGTGTACCAGTCTGAGGCCCATGGAC
TCCCCCTGCATCTGCCCGGCAACAAGTCCCCTCACCGGGATCCTGCCCCAAGAGGCCCAGC
TCGGTTTCTGCCTCTGCCGGGACTGCCTCCAGCGTTGCCCGAACCCCCTGGTATCCTGGCC
CCGCAACCACCTGACGTCGGTTCGTCGGACCCGCTGAGCATGGTCGGTCCGAGCCAGGGA
AGGTCCCCGTCCTACGCATCCTGA
Codon optimized nucleotide sequence of homo sapiens FGF21-variant 3 (SEQ ID NO: 7)
ATGGATTCCGACGAAACTGGATTTGAACATTCAGGGCTGTGGGTCTCTGTGCTGGCTGGACT
GCTGCTGGGGGCTTGTCAGGCTCACCCCATCCCTGACAGCTCCCCTCTGCTGCAGTTCGGA
GGACAGGTGCGGCAGAGATACCTGTATACCGACGATGCCCAGCAGACAGAGGCACACCTG
GAGATCAGGGAGGACGGAACCGTGGGAGGAGCAGCCGATCAGTCTCCCGAGAGCCTGCTG
CAGCTGAAGGCCCTGAAGCCTGGCGTGATCCAGATCCTGGGCGTGAAGACATCTCGGTTTC
TGTGCCAGCGGCCCGACGGCGCCCTGTACGGCTCCCTGCACTTCGATCCCGAGGCCTGTT
CTTTTAGGGAGCTGCTGCTGGAGGACGGCTACAACGTGTATCAGAGCGAGGCACACGGCCT
GCCACTGCACCTGCCTGGCAATAAGTCCCCTCACCGCGATCCAGCACCCAGGGGCCCAGCA
CGCTTCCTGCCTCTGCCAGGCCTGCCCCCTGCCCTGCCAGAGCCACCCGGCATCCTGGCC
CCCCAGCCTCCAGATGTGGGCTCCAGCGATCCTCTGTCAATGGTGGGGCCAAGTCAGGGG
CGGAGTCCTTCATACGCATCATAA
Nucleotide sequence of murine codon-optimized FGF21 (SEQ ID NO: 9)
ATGGAATGGATGAGAAGCAGAGTGGGCACCCTGGGCCTGTGGGTGCGACTGCTGCTGGCT
GTGTTTCTGCTGGGCGTGTACCAGGCCTACCCCATCCCTGACTCTAGCCCCCTGCTGCAGTT
TGGCGGACAAGTGCGGCAGAGATACCTGTACACCGACGACGACCAGGACACCGAGGCCCA
CCTGGAAATCCGCGAGGATGGCACAGTCGTGGGCGCTGCTCACAGAAGCCCTGAGAGCCT
GCTGGAACTGAAGGCCCTGAAGCCCGGCGTGATCCAGATCCTGGGCGTGAAGGCCAGCAG
ATTCCTGTGCCAGCAGCCTGACGGCGCCCTGTACGGCTCTCCTCACTTCGATCCTGAGGCC
TGCAGCTTCAGAGAGCTGCTGCTGGAGGACGGCTACAACGTGTACCAGTCTGAGGCCCACG
GCCTGCCCCTGAGACTGCCTCAGAAGGACAGCCCTAACCAGGACGCCACAAGCTGGGGAC
CTGTGCGGTTCCTGCCTATGCCTGGACTGCTGCACGAGCCCCAGGATCAGGCTGGCTTTCT
GCCTCCTGAGCCTCCAGACGTGGGCAGCAGCGACCCTCTGAGCATGGTGGAACCTCTGCA
GGGCAGAAGCCCCAGCTACGCCTCTTGA
Nucleotide sequence of CAG promoter (SEQ ID NO: 27)
GACATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATC
ATATGCCAAGTACGCCCCCTATTGACGTCAATGACGGTAAATGGCCCGCCTGGCATTATGCC
CAGTACATGACCTTATGGGACTTTCCTACTTGGCAGTACATCTACGTATTAGTCATCGCTATT
ACCATGGTCGAGGTGAGCCCCACGTTCTGCTTCACTCTCCCCATCTCCCCCCCCTCCCCAC
CCCCAATTTTGTATTTATTTATTTTTTAATTATTTTGTGCAGCGATGGGGGCGGGGGGGGGGG
GGGGGCGCGCGCCAGGCGGGGCGGGGCGGGGCGAGGGGCGGGGCGGGGCGAGGCGGA
GAGGTGCGGCGGCAGCCAATCAGAGCGGCGCGCTCCGAAAGTTTCCTTTTATGGCGAGGC
GGCGGCGGCGGCGGCCCTATAAAAAGCGAAGCGCGCGGCGGGCGGGAGTCGCTGCGTTG
CCTTCGCCCCGTGCCCCGCTCCGCGCCGCCTCGCGCCGCCCGCCCCGGCTCTGACTGACC
GCGTTACTCCCACAGGTGAGCGGGCGGGACGGCCCTTCTCCTCCGGGCTGTAATTAGCGCT
TGGTTTAATGACGGCTTGTTTCTTTTCTGTGGCTGCGTGAAAGCCTTGAGGGGCTCCGGGAG
GGCCCTTTGTGCGGGGGGAGCGGCTCGGGGGGTGCGTGCGTGTGTGTGTGCGTGGGGAG
CGCCGCGTGCGGCTCCGCGCTGCCCGGCGGCTGTGAGCGCTGCGGGCGCGGCGCGGGG
CTTTGTGCGCTCCGCAGTGTGCGCGAGGGGAGCGCGGCCGGGGGCGGTGCCCCGCGGTG
CGGGGGGCTGCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCCCCGAGTTGCT
GAGCACGGCCCGGCTTCGGGTGCGGGGCTCCGTACGGGGCGTGGCGCGGGGCTCGCCGT
GCCGGGCGGGGGGTGGCGGCAGGTGGGGGTGCCGGGCGGGGCGGGGCCGCCTCGGGC
CGGGGAGGGCTCGGGGGAGGGGCGCGGCGGCCCCCGGAGCGCCGGCGGCTGTCGAGGC
GCGGCGAGCCGCAGCCATTGCCTTTTATGGTAATCGTGCGAGAGGGCGCAGGGACTTCCTT
TGTCCCAAATCTGTGCGGAGCCGAAATCTGGGAGGCGCCGCCGCACCCCCTCTAGCGGGC
GCGGGGCGAAGCGGTGCGGCGCCGGCAGGAAGGAAATGGGCGGGGAGGGCCTTCGTGCG
TCGCCGCGCCGCCGTCCCCTTCTCCCTCTCCAGCCTCGGGGCTGTCCGCGGGGGGACGGC
TGCCTTCGGGGGGGACGGGGCAGGGCGGGGTTCGGCTTCTGGCGTGTGACCGGCGGCTC
TAGAGCCTCTGCTAACCATGTTCATGCCTTCTTCTTTTTCCTACAG
Nucleotide sequence of CMV promoter (SEQ ID NO: 28)
GTGATGCGGTTTTGGCAGTACACCAATGGGCGTGGATAGCGGTTTGACTCACGGGGATTTC
CAAGTCTCCACCCCATTGACGTCAATGGGAGTTTGTTTTGGCACCAAAATCAACGGGACTTT
CCAAAATGTCGTAACAACTGCGATCGCCCGCCCCGTTGACGCAAATGGGCGGTAGGCGTGT
ACGGTGGGAGGTCTATATAAGCAGAGCT
Nucleotide sequence of CMV enhancer (SEQ ID NO: 29)
GGCATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATC
ATATGCCAAGTCCGCCCCCTATTGACGTCAATGACGGTAAATGGCCCGCCTGGCATTATGCC
CAGTACATGACCTTACGGGACTTTCCTACTTGGCAGTACATCTACGTATTAGTCATCGCTATT
ACCATG
CMV promoter and CMV enhancer sequence (SEQ ID NO: 34)
GGCATTGATTATTGACTAGTTATTAATAGTAATCAATTACGGGGTCATTAGTTCATAGCCCATA
TATGGAGTTCCGCGTTACATAACTTACGGTAAATGGCCCGCCTGGCTGACCGCCCAACGAC
CCCCGCCCATTGACGTCAATAATGACGTATGTTCCCATAGTAACGCCAATAGGGACTTTCCA
TTGACGTCAATGGGTGGAGTATTTACGGTAAACTGCCCACTTGGCAGTACATCAAGTGTATC
ATATGCCAAGTCCGCCCCCTATTGACGTCAATGACGGTAAATGGCCCGCCTGGCATTATGCC
CAGTACATGACCTTACGGGACTTTCCTACTTGGCAGTACATCTACGTATTAGTCATCGCTATT
ACCATGGTGATGCGGTTTTGGCAGTACACCAATGGGCGTGGATAGCGGTTTGACTCACGGG
GATTTCCAAGTCTCCACCCCATTGACGTCAATGGGAGTTTGTTTTGGCACCAAAATCAACGG
GACTTTCCAAAATGTCGTAACAACTGCGATCGCCCGCCCCGTTGACGCAAATGGGCGGTAG
GCGTGTACGGTGGGAGGTCTATATAAGCAGAGCT
AAV2 5′ ITR (SEQ ID NO: 30)
GCGCGCTC GCTCGCTCAC TGAGGCCGCC CGGGCAAAGC
CCGGGCGTCG GGCGACCTTT GGTCGCCCGG CCTCAGTGAG CGAGCGAGCG
CGCAGAGAGG GAGTGGCCAA CTCCATCACT AGGGGTTCCT
AAV2 3′ ITR (SEQ ID NO: 31)
AGGAACCCCT AGTGATGGAG TTGGCCACTC CCTCTCTGCG
CGCTCGCTCG CTCACTGAGG CCGGGCGACC AAAGGTCGCC CGACGCCCGG
GCTTTGCCCG GGCGGCCTCA GTGAGCGAGC GAGCGCGC
Rabbit β-globin polyadenylation signal
(3′ UTR and flanking region of rabbit beta-globin, including
polyA signal) (SEQ ID NO: 33)
GATCTTTTTCCCTCTGCCAAAAATTATGGGGACATCATGAAGCCCCTTGAGCATCTGACTTCT
GGCTAATAAAGGAAATTTATTTTCATTGCAATAGTGTGTTGGAATTTTTTGTGTCTCTCACTCG
GAAGGACATATGGGAGGGCAAATCATTTAAAACATCAGAATGAGTATTTGGTTTAGAGTTTGG
CAACATATGCCCATATGCTGGCTGCCATGAACAAAGGTTGGCTATAAAGAGGTCATCAGTAT
ATGAAACAGCCCCCTGCTGTCCATTCCTTATTCCATAGAAAAGCCTTGACTTGAGGTTAGATT
TTTTTTATATTTTGTTTTGTGTTATTTTTTTCTTTAACATCCCTAAAATTTTCCTTACATGTTTTAC
TAGCCAGATTTTTCCTCCTCTCCTGACTACTCCCAGTCATAGCTGTCCCTCTTCTCTTATGGA
GATC
miRT seguences
miRT-122a (SEQ ID NO: 12):
5′ CAAACACCATTGTCACACTCCA 3′,
target for the microRNA-122a (Accession Number to the miRBase database MI0000442),
which is expressed in the liver.
miRT-152 (SEQ ID NO: 14):
5′ CCAAGTTCTGTCATGCACTGA 3′,
target for the microRNA-152 (MI0000462), which is expressed in the liver.
miRT-199a-5p (SEQ ID NO: 15):
5′ GAACAGGTAGTCTGAACACTGGG 3′,
target for the microRNA 199a (MI0000242), which is expressed in the liver.
miRT-199a-3p (SEQ ID NO: 16):
5′ TAACCAATGTGCAGACTACTGT 3′,
target for the microRNA-199a (MI0000242), which is expressed in the liver.
miRT-215 (SEQ ID NO: 17):
5′ GTCTGTCAATTCATAGGTCAT 3′,
target for the microRNA-215 (MI0000291), which is expressed in the liver.
miRT-192 (SEQ ID NO: 18):
5′ GGCTGTCAATTCATAGGTCAG 3′,
target for the microRNA-192 (MI0000234), which is expressed in the liver.
miRT-148a (SEQ ID NO: 19):
5′ ACAAAGTTCTGTAGTGCACTGA 3′,
target for the microRNA-148a (MI0000253), which is expressed in the liver.
miRT-194 (SEQ ID NO: 20):
5′ TCCACATGGAGTTGCTGTTACA 3′,
target for the microRNA-194 (MI0000488), which is expressed in the liver.
miRT-133a (SEQ ID NO: 21):
5′ CAGCTGGTTGAAGGGGACCAAA 3′,
target for the microRNA-133a (MI0000450), which is expressed in the heart.
miRT-206 (SEQ ID NO: 22):
5′ CCACACACTTCCTTACATTCCA 3′,
target for the microRNA-206 (MI0000490), which is expressed in the heart.
miRT-1 (SEQ ID NO: 13):
5′ TTACATACTTCTTTACATTCCA 3′,
target for the microRNA-1 (MI0000651), which is expressed in the heart.
miRT-208a-5p (SEQ ID NO: 23):
5′ GTATAACCCGGGCCAAAAGCTC 3′,
target for the microRNA-208a (MI0000251), which is expressed in the heart.
miR1-208a-3p (SEQ ID NO: 24):
5′ ACAAGCTTTTTGCTCGTCTTAT 3′,
target for the microRNA-208a (MI0000251), which is expressed in the heart.
miRT-499-5p (SEQ ID NO: 25):
5′ AAACATCACTGCAAGTCTTAA 3′,
target for the microRNA-499 (MI0003183), which is expressed in the heart.
pAAV-CAG-moFGF21-dmiRT (SEQ ID NO: 35)

| | |
|---|---|
| 1 | AGTGAGCGAG CGAGCGCGCA GCTGCATTAA TGAATCGGCC AACGCGCGGG |
| 51 | GAGAGGCGGT TTGCGTATTG GGCGCTCTTC CGCTTCCTCG CTCACTGACT |
| 101 | CGCTGCGCTC GGTCGTTCGG CTGCGGCGAG CGGTATCAGC TCACTCAAAG |
| 151 | GCGGTAATAC GGTTATCCAC AGAATCAGGG GATAACGCAG GAAAGAACAT |
| 201 | GTGAGCAAAA GGCCAGCAAA AGGCCAGGAA CCGTAAAAAG GCCGCGTTGC |
| 251 | TGGCGTTTTT CCATAGGCTC CGCCCCCCTG ACGAGCATCA CAAAAATCGA |
| 301 | CGCTCAAGTC AGAGGTGGCG AAACCCGACA GGACTATAAA GATACCAGGC |
| 351 | GTTTCCCCCT GGAAGCTCCC TCGTGCGCTC TCCTGTTCCG ACCCTGCCGC |
| 401 | TTACCGGATA CCTGTCCGCC TTTCTCCCTT CGGGAAGCGT GGCGCTTTCT |
| 451 | CATAGCTCAC GCTGTAGGTA TCTCAGTTCG GTGTAGGTCG TTCGCTCCAA |
| 501 | GCTGGGCTGT GTGCACGAAC CCCCCGTTCA GCCCGACCGC TGCGCCTTAT |
| 551 | CCGGTAACTA TCGTCTTGAG TCCAACCCGG TAAGACACGA CTTATCGCCA |
| 601 | CTGGCAGCAG CCACTGGTAA CAGGATTAGC AGAGCGAGGT ATGTAGGCGG |
| 651 | TGCTACAGAG TTCTTGAAGT GGTGGCCTAA CTACGGCTAC ACTAGAAGAA |
| 701 | CAGTATTTGG TATCTGCGCT CTGCTGAAGC CAGTTACCTT CGGAAAAAGA |
| 751 | GTTGGTAGCT CTTGATCCGG CAAACAAACC ACCGCTGGTA GCGGTGGTTT |
| 801 | TTTTGTTTGC AAGCAGCAGA TTACGCGCAG AAAAAAAGGA TCTCAAGAAG |
| 851 | ATCCTTTGAT CTTTTCTACG GGGTCTGACG CTCAGTGGAA CGAAAACTCA |
| 901 | CGTTAAGGGA TTTTGGTCAT GAGATTATCA AAAAGGATCT TCACCTAGAT |
| 951 | CCTTTTAAAT TAAAAATGAA GTTTTAAATC AATCTAAAGT ATATATGAGT |
| 1001 | AAACTTGGTC TGACAGTTAC CAATGCTTAA TCAGTGAGGC ACCTATCTCA |
| 1051 | GCGATCTGTC TATTTCGTTC ATCCATAGTT GCCTGACTCC CCGTCGTGTA |
| 1101 | GATAACTACG ATACGGGAGG GCTTACCATC TGGCCCCAGT GCTGCAATGA |
| 1151 | TACCGCGAGA CCCACGCTCA CCGGCTCCAG ATTTATCAGC AATAAACCAG |
| 1201 | CCAGCCGGAA GGGCCGAGCG CAGAAGTGGT CCTGCAACTT TATCCGCCTC |
| 1251 | CATCCAGTCT ATTAATTGTT GCCGGGAAGC TAGAGTAAGT AGTTCGCCAG |
| 1301 | TTAATAGTTT GCGCAACGTT GTTGCCATTG CTACAGGCAT CGTGGTGTCA |
| 1351 | CGCTCGTCGT TTGGTATGGC TTCATTCAGC TCCGGTTCCC AACGATCAAG |
| 1401 | GCGAGTTACA TGATCCCCCA TGTTGTGCAA AAAAGCGGTT AGCTCCTTCG |
| 1451 | GTCCTCCGAT CGTTGTCAGA AGTAAGTTGG CCGCAGTGTT ATCACTCATG |
| 1501 | GTTATGGCAG CACTGCATAA TTCTCTTACT GTCATGCCAT CCGTAAGATG |
| 1551 | CTTTTCTGTG ACTGGTGAGT ACTCAACCAA GTCATTCTGA GAATAGTGTA |
| 1601 | TGCGGCGACC GAGTTGCTCT TGCCCGGCGT CAATACGGGA TAATACCGCG |
| 1651 | CCACATAGCA GAACTTTAAA AGTGCTCATC ATTGGAAAAC GTTCTTCGGG |
| 1701 | GCGAAAACTC TCAAGGATCT TACCGCTGTT GAGATCCAGT TCGATGTAAC |
| 1751 | CCACTCGTGC ACCCAACTGA TCTTCAGCAT CTTTTACTTT CACCAGCGTT |
| 1801 | TCTGGGTGAG CAAAAACAGG AAGGCAAAAT GCCGCAAAAA AGGGAATAAG |
| 1851 | GGCGACACGG AAATGTTGAA TACTCATACT CTTCCTTTTT CAATATTATT |
| 1901 | GAAGCATTTA TCAGGGTTAT TGTCTCATGA GCGGATACAT ATTTGAATGT |
| 1951 | ATTTAGAAAA ATAAACAAAT AGGGGTTCCG CGCACATTTC CCCGAAAAGT |
| 2001 | GCCACCTGAC GTCTAAGAAA CCATTATTAT CATGACATTA ACCTATAAAA |
| 2051 | ATAGGCGTAT CACGAGGCCC TTTCGTCTCG CGCGTTTCGG TGATGACGGT |
| 2101 | GAAAACCTCT GACACATGCA GCTCCCGGAG ACGGTCACAG CTTGTCTGTA |
| 2151 | AGCGGATGCC GGGAGCAGAC AAGCCCGTCA GGGCGCGTCA GCGGGTGTTG |
| 2201 | GCGGGTGTCG GGGCTGGCTT AACTATGCGG CATCAGAGCA GATTGTACTG |
| 2251 | AGAGTGCACC ATATGCGGTG TGAAATACCG CACAGATGCG TAAGGAGAAA |
| 2301 | ATACCGCATC AGGCGATTCC AACATCCAAT AAATCATACA GGCAAGGCAA |
| 2351 | AGAATTAGCA AAATTAAGCA ATAAAGCCTC AGAGCATAAA GCTAAATCGG |
| 2401 | TTGTACCAAA AACATTATGA CCCTGTAATA CTTTTGCGGG AGAAGCCTTT |
| 2451 | ATTTCAACGC AAGGATAAAA ATTTTTAGAA CCCTCATATA TTTTAAATGC |
| 2501 | AATGCCTGAG TAATGTGTAG GTAAAGATTC AAACGGGTGA GAAAGGCCGG |
| 2551 | AGACAGTCAA ATCACCATCA ATATGATATT CAACCGTTCT AGCTGATAAA |
| 2601 | TTCATGCCGG AGAGGGTAGC TATTTTTGAG AGGTCTCTAC AAAGGCTATC |
| 2651 | AGGTCATTGC CTGAGAGTCT GGAGCAAACA AGAGAATCGA TGAACGGTAA |
| 2701 | TCGTAAAACT AGCATGTCAA TCATATGTAC CCCGGTTGAT AATCAGAAAA |
| 2751 | GCCCCAAAAA CAGGAAGATT GTATAAGCAA ATATTTAAAT TGTAAGCGTT |
| 2801 | AATATTTTGT TAAAATTCGC GTTAAATTTT TGTTAAATCA GCTCATTTTT |
| 2851 | TAACCAATAG GCCGAAATCG GCAAAATCCC TTATAAATCA AAAGAATAGA |
| 2901 | CCGAGATAGG GTTGAGTGTT GTTCCAGTTT GGAACAAGAG TCCACTATTA |
| 2951 | AAGAACGTGG ACTCCAACGT CAAAGGGCGA AAAACCGTCT ATCAGGGCGA |
| 3001 | TGGCCCACTA CGTGAACCAT CACCCTAATC AAGTTTTTTG GGGTCGAGGT |
| 3051 | GCCGTAAAGC ACTAAATCGG AACCCTAAAG GGAGCCCCCG ATTTAGAGCT |
| 3101 | TGACGGGGAA AGCCGGCGAA CGTGGCGAGA AAGGAAGGGA AGAAAGCGAA |
| 3151 | AGGAGCGGGC GCTAGGGCGC TGGCAAGTGT AGCGGTCACG CTGCGCGTAA |
| 3201 | CCACCACACC CGCCGCGCTT AATGCGCCGC TACAGGGCGC GTACTATGGT |
| 3251 | TGCTTTGACG AGCACGTATA ACGTGCTTTC CTCGTTAGAA TCAGAGCGGG |
| 3301 | AGCTAAACAG GAGGCCGATT AAAGGGATTT TAGACAGGAA CGGTACGCCA |
| 3351 | GAATCCTGAG AAGTGTTTTT ATAATCAGTG AGGCCACCGA GTAAAAGAGT |
| 3401 | CTGTCCATCA CGCAAATTAA CCGTTGTCGC AATACTTCTT TGATTAGTAA |
| 3451 | TAACATCACT TGCCTGAGTA GAAGAACTCA AACTATCGGC CTTGCTGGTA |
| 3501 | ATATCCAGAA CAATATTACC GCCAGCCATT GCAACGGAAT CGCCATTCGC |
| 3551 | CATTCAGGCT GCGCAACTGT TGGGAAGGGC GATCGGTGCG GGCCTCTTCC |
| 3601 | ACTGAGGCCC AGCTGCGCGC TCGCTCGCTC ACTGAGGCCG CCCGGGCAAA |
| 3651 | GCCCGGGCGT CGGGCGACCT TTGGTCGCCC GGCCTCAGTG AGCGAGCGAG |
| 3701 | CGCGCAGAGA GGGAGTGGCC AACTCCATCA CTAGGGGTTC CTTGTAGTTA |
| 3751 | ATGATTAACC CGCCATGCTA CTTATCTACT CGACATTGAT TATTGACTAG |
| 3801 | TTATTAATAG TAATCAATTA CGGGGTCATT AGTTCATAGC CCATATATGG |
| 3851 | AGTTCCGCGT TACATAACTT ACGGTAAATG GCCCGCCTGG CTGACCGCCC |
| 3901 | AACGACCCCC GCCCATTGAC GTCAATAATG ACGTATGTTC CCATAGTAAC |
| 3951 | GCCAATAGGG ACTTTCCATT GACGTCAATG GGTGGAGTAT TTACGGTAAA |
| 4001 | CTGCCCACTT GGCAGTACAT CAAGTGTATC ATATGCCAAG TACGCCCCCT |
| 4051 | ATTGACGTCA ATGACGGTAA ATGGCCCGCC TGGCATTATG CCCAGTACAT |
| 4101 | GACCTTATGG GACTTTCCTA CTTGGCAGTA CATCTACGTA TTAGTCATCG |
| 4151 | CTATTACCAT GGTCGAGGTG AGCCCCACGT TCTGCTTCAC TCTCCCCATC |
| 4201 | TCCCCCCCCT CCCCACCCCC AATTTTGTAT TTATTTATTT TTTAATTATT |
| 4251 | TTGTGCAGCG ATGGGGGCGG GGGGGGGGGG GGGGCGCGCG CCAGGCGGGG |
| 4301 | CGGGGCGGGG CGAGGGGCGG GGCGGGGCGA GGCGGAGAGG TGCGGCGGCA |
| 4351 | GCCAATCAGA GCGGCGCGCT CCGAAAGTTT CCTTTTATGG CGAGGCGGCG |
| 4401 | GCGGCGGCGG CCCTATAAAA AGCGAAGCGC GCGGCGGGCG GGAGTCGCTG |
| 4451 | CGTTGCCTTC GCCCCGTGCC CCGCTCCGCG CCGCCTCGCG CCGCCCGCCC |
| 4501 | CGGCTCTGAC TGACCGCGTT ACTCCCACAG GTGAGCGGGC GGGACGGCCC |
| 4551 | TTCTCCTCCG GGCTGTAATT AGCGCTTGGT TTAATGACGG CTTGTTTCTT |
| 4601 | TTCTGTGGCT GCGTGAAAGC CTTGAGGGGC TCCGGGAGGG CCCTTTGTGC |
| 4651 | GGGGGGAGCG GCTCGGGGGG TGCGTGCGTG TGTGTGTGCG TGGGGAGCGC |
| 4701 | CGCGTGCGGC TCCGCGCTGC CCGGCGGCTG TGAGCGCTGC GGGCGCGGCG |
| 4751 | CGGGGCTTTG TGCGCTCCGC AGTGTGCGCG AGGGGAGCGC GGCCGGGGGC |
| 4801 | GGTGCCCCGC GGTGCGGGGG GCTGCGAGGG GAACAAAGGC TGCGTGCGGG |
| 4851 | GTGTGTGCGT GGGGGGGTGA GCAGGGGGTG TGGGCGCGTC GGTCGGGCTG |
| 4901 | CAACCCCCCC TGCACCCCCC TCCCCGAGTT GCTGAGCACG GCCCGGCTTC |
| 4951 | GGGTGCGGGG CTCCGTACGG GGCGTGGCGC GGGGCTCGCC GTGCCGGGCG |
| 5001 | GGGGGTGGCG GCAGGTGGGG GTGCCGGGCG GGGCGGGGCC GCCTCGGGCC |
| 5051 | GGGGAGGGCT CGGGGGAGGG GCGCGGCGGC CCCCGGAGCG CCGGCGGCTG |
| 5101 | TCGAGGCGCG GCGAGCCGCA GCCATTGCCT TTTATGGTAA TCGTGCGAGA |
| 5151 | GGGCGCAGGG ACTTCCTTTG TCCCAAATCT GTGCGGAGCC GAAATCTGGG |
| 5201 | AGGCGCCGCC GCACCCCCTC TAGCGGGCGC GGGGCGAAGC GGTGCGGCGC |
| 5251 | CGGCAGGAAG GAAATGGGCG GGGAGGGCCT TCGTGCGTCG CCGCGCCGCC |
| 5301 | GTCCCCTTCT CCCTCTCCAG CCTCGGGGCT GTCCGCGGGG GGACGGCTGC |
| 5351 | CTTCGGGGGG GACGGGGCAG GGCGGGGTTC GGCTTCTGGC GTGTGACCGG |
| 5401 | CGGCTCTAGA GCCTCTGCTA ACCATGTTCA TGCCTTCTTC TTTTTCCTAC |
| 5451 | AGCTCCTGGG CAACGTGCTG GTTATTGTGC TGTCTCATCA TTTTGGCAAA |
| 5501 | GAATTGATTA ATTCGAGCGA ACGCGTCGAG TCGCTCGGTA CGATTTAAAT |
| 5551 | TGAATTGGCC TCGAGCGCAA GCTTGAGCTA GCGCCACCAT GGAATGGATG |
| 5601 | AGAAGCAGAG TGGGCACCCT GGGCCTGTGG GTGCGACTGC TGCTGGCTGT |
| 5651 | GTTTCTGCTG GGCGTGTACC AGGCCTACCC CATCCCTGAC TCTAGCCCCC |
| 5701 | TGCTGCAGTT TGGCGGACAA GTGCGGCAGA GATACCTGTA CACCGACGAC |
| 5751 | GACCAGGACA CCGAGGCCCA CCTGGAAATC CGCGAGGATG GCACAGTCGT |
| 5801 | GGGCGCTGCT CACAGAAGCC CTGAGAGCCT GCTGGAACTG AAGGCCCTGA |
| 5851 | AGCCCGGCGT GATCCAGATC CTGGGCGTGA AGGCCAGCAG ATTCCTGTGC |
| 5901 | CAGCAGCCTG ACGGCGCCCT GTACGGCTCT CCTCACTTCG ATCCTGAGGC |
| 5951 | CTGCAGCTTC AGAGAGCTGC TGCTGGAGGA CGGCTACAAC GTGTACCAGT |
| 6001 | CTGAGGCCCA CGGCCTGCCC CTGAGACTGC CTCAGAAGGA CAGCCCTAAC |
| 6051 | CAGGACGCCA CAAGCTGGGG ACCTGTGCGG TTCCTGCCTA TGCCTGGACT |
| 6101 | GCTGCACGAG CCCCAGGATC AGGCTGGCTT TCTGCCTCCT GAGCCTCCAG |
| 6151 | ACGTGGGCAG CAGCGACCCT CTGAGCATGG TGGAACCTCT GCAGGGCAGA |
| 6201 | AGCCCCAGCT ACGCCTCTTG AGAATGCGGG CCCGGTACCC CCGACGCGGC |
| 6251 | CGCTAATTCT AGATCGCGAA CAAACACCAT TGTCACACTC CAGTATACAC |
| 6301 | AAACACCATT GTCACACTCC AGATATCACA AACACCATTG TCACACTCCA |
| 6351 | AGGCGAACAA ACACCATTGT CACACTCCAA GGCTATTCTA GATCGCGAAT |
| 6401 | TACATACTTC TTTACATTCC AGTATACATT ACATACTTCT TTACATTCCA |
| 6451 | GATATCATTA CATACTTCTT TACATTCCAA GGCGAATTAC ATACTTCTTT |
| 6501 | ACATTCCAAG GCTACCTGAG GCCCGGGGGT ACCTCTTAAT TAACTGGCCT |
| 6551 | CATGGGCCTT CCGCTCACTG CCCGCTTTCC AGTCGGGAAA CCTGTCGTGC |
| 6601 | CAGTCAGGTG CAGGCTGCCT ATCAGAAGGT GGTGGCTGGT GTGGCCAATG |
| 6651 | CCCTGGCTCA CAAATACCAC TGAGATCTTT TTCCCTCTGC CAAAAATTAT |
| 6701 | GGGGACATCA TGAAGCCCCT TGAGCATCTG ACTTCTGGCT AATAAAGGAA |
| 6751 | ATTTATTTTC ATTGCAATAG TGTGTTGGAA TTTTTTGTGT CTCTCACTCG |
| 6801 | GAAGGACATA TGGGAGGGCA AATCATTTAA AACATCAGAA TGAGTATTTG |
| 6851 | GTTTAGAGTT TGGCAACATA TGCCCATATG CTGGCTGCCA TGAACAAAGG |
| 6901 | TTGGCTATAA AGAGGTCATC AGTATATGAA ACAGCCCCCT GCTGTCCATT |
| 6951 | CCTTATTCCA TAGAAAAGCC TTGACTTGAG GTTAGATTTT TTTTATATTT |
| 7001 | TGTTTTGTGT TATTTTTTTC TTTAACATCC CTAAAATTTT CCTTACATGT |
| 7051 | TTTACTAGCC AGATTTTTCC TCCTCTCCTG ACTACTCCCA GTCATAGCTG |
| 7101 | TCCCTCTTCT CTTATGGAGA TCCCTCGACC TGCAGCCCAA GCTGTAGATA |
| 7151 | AGTAGCATGG CGGGTTAATC ATTAACTACA AGGAACCCCT AGTGATGGAG |
| 7201 | TTGGCCACTC CCTCTCTGCG CGCTCGCTCG CTCACTGAGG CCGGGCGACC |
| 7251 | AAAGGTCGCC CGACGCCCGG GCTTTGCCCG GGCGGCCTCA GTGAGCGAGC |
| 7301 | GAGCGCGCAG CTGGCGTAA |

AAV2 5′ ITR: 3615-3742 bp
CAG promoter: 3782-5452 bp
Mus musculus codon-optimized FGF21 (moFGF21): 5589-6221 bp
dmiRT (4 copies of the miRT-122a and 4 copies of the miRT-1): 6254-6514 bp
Rabbit β-globin polyA signal (3′ UTR and 3′ flanking region of rabbit beta-globin,
including polyA signal): 6674-6764 bp
AAV2 3′ ITR: 7181-7308 bp
pAAV-CAG-moFGF21 (SEQ ID NO: 46)

| | |
|---|---|
| 1 | AGTGAGCGAG CGAGCGCGCA GCTGCATTAA TGAATCGGCC AACGCGCGGG GAGAGGCGGT |
| 61 | TTGCGTATTG GGCGCTCTTC CGCTTCCTCG CTCACTGACT CGCTGCGCTC GGTCGTTCGG |
| 121 | CTGCGGCGAG CGGTATCAGC TCACTCAAAG GCGGTAATAC GGTTATCCAC AGAATCAGGG |
| 181 | GATAACGCAG GAAAGAACAT GTGAGCAAAA GGCCAGCAAA AGGCCAGGAA CCGTAAAAAG |
| 241 | GCCGCGTTGC TGGCGTTTTT CCATAGGCTC CGCCCCCCTG ACGAGCATCA CAAAAATCGA |
| 301 | CGCTCAAGTC AGAGGTGGCG AAACCCGACA GGACTATAAA GATACCAGGC GTTTCCCCCT |
| 361 | GGAAGCTCCC TCGTGCGCTC TCCTGTTCCG ACCCTGCCGC TTACCGGATA CCTGTCCGCC |
| 421 | TTTCTCCCTT CGGGAAGCGT GGCGCTTTCT CATAGCTCAC GCTGTAGGTA TCTCAGTTCG |
| 481 | GTGTAGGTCG TTCGCTCCAA GCTGGGCTGT GTGCACGAAC CCCCCGTTCA GCCCGACCGC |
| 541 | TGCGCCTTAT CCGGTAACTA TCGTCTTGAG TCCAACCCGG TAAGACACGA CTTATCGCCA |
| 601 | CTGGCAGCAG CCACTGGTAA CAGGATTAGC AGAGCGAGGT ATGTAGGCGG TGCTACAGAG |
| 661 | TTCTTGAAGT GGTGGCCTAA CTACGGCTAC ACTAGAAGAA CAGTATTTGG TATCTGCGCT |
| 721 | CTGCTGAAGC CAGTTACCTT CGGAAAAAGA GTTGGTAGCT CTTGATCCGG CAAACAAACC |
| 781 | ACCGCTGGTA GCGGTGGTTT TTTTGTTTGC AAGCAGCAGA TTACGCGCAG AAAAAAAGGA |
| 841 | TCTCAAGAAG ATCCTTTGAT CTTTTCTACG GGGTCTGACG CTCAGTGGAA CGAAAACTCA |
| 901 | CGTTAAGGGA TTTTGGTCAT GAGATTATCA AAAAGGATCT TCACCTAGAT CCTTTTAAAT |
| 961 | TAAAAATGAA GTTTTAAATC AATCTAAAGT ATATATGAGT AAACTTGGTC TGACAGTTAC |
| 1021 | CAATGCTTAA TCAGTGAGGC ACCTATCTCA GCGATCTGTC TATTTCGTTC ATCCATAGTT |
| 1081 | GCCTGACTCC CCGTCGTGTA GATAACTACG ATACGGGAGG GCTTACCATC TGGCCCCAGT |
| 1141 | GCTGCAATGA TACCGCGAGA CCCACGCTCA CCGGCTCCAG ATTTATCAGC AATAAACCAG |
| 1201 | CCAGCCGGAA GGGCCGAGCG CAGAAGTGGT CCTGCAACTT TATCCGCCTC CATCCAGTCT |
| 1261 | ATTAATTGTT GCCGGGAAGC TAGAGTAAGT AGTTCGCCAG TTAATAGTTT GCGCAACGTT |
| 1321 | GTTGCCATTG CTACAGGCAT CGTGGTGTCA CGCTCGTCGT TTGGTATGGC TTCATTCAGC |
| 1381 | TCCGGTTCCC AACGATCAAG GCGAGTTACA TGATCCCCCA TGTTGTGCAA AAAAGCGGTT |
| 1441 | AGCTCCTTCG GTCCTCCGAT CGTTGTCAGA AGTAAGTTGG CCGCAGTGTT ATCACTCATG |
| 1501 | GTTATGGCAG CACTGCATAA TTCTCTTACT GTCATGCCAT CCGTAAGATG CTTTTCTGTG |
| 1561 | ACTGGTGAGT ACTCAACCAA GTCATTCTGA GAATAGTGTA TGCGGCGACC GAGTTGCTCT |
| 1621 | TGCCCGGCGT CAATACGGGA TAATACCGCG CCACATAGCA GAACTTTAAA AGTGCTCATC |
| 1681 | ATTGGAAAAC GTTCTTCGGG GCGAAAACTC TCAAGGATCT TACCGCTGTT GAGATCCAGT |
| 1741 | TCGATGTAAC CCACTCGTGC ACCCAACTGA TCTTCAGCAT CTTTTACTTT CACCAGCGTT |
| 1801 | TCTGGGTGAG CAAAAACAGG AAGGCAAAAT GCCGCAAAAA AGGGAATAAG GGCGACACGG |
| 1861 | AAATGTTGAA TACTCATACT CTTCCTTTTT CAATATTATT GAAGCATTTA TCAGGGTTAT |
| 1921 | TGTCTCATGA GCGGATACAT ATTTGAATGT ATTTAGAAAA ATAAACAAAT AGGGGTTCCG |
| 1981 | CGCACATTTC CCCGAAAAGT GCCACCTGAC GTCTAAGAAA CCATTATTAT CATGACATTA |
| 2041 | ACCTATAAAA ATAGGCGTAT CACGAGGCCC TTTCGTCTCG CGCGTTTCGG TGATGACGGT |
| 2101 | GAAAACCTCT GACACATGCA GCTCCCGGAG ACGGTCACAG CTTGTCTGTA AGCGGATGCC |
| 2161 | GGGAGCAGAC AAGCCCGTCA GGGCGCGTCA GCGGGTGTTG GCGGGTGTCG GGGCTGGCTT |
| 2221 | AACTATGCGG CATCAGAGCA GATTGTACTG AGAGTGCACC ATATGCGGTG TGAAATACCG |
| 2281 | CACAGATGCG TAAGGAGAAA ATACCGCATC AGGCGATTCC AACATCCAAT AAATCATACA |
| 2341 | GGCAAGGCAA AGAATTAGCA AAATTAAGCA ATAAAGCCTC AGAGCATAAA GCTAAATCGG |
| 2401 | TTGTACCAAA AACATTATGA CCCTGTAATA CTTTTGCGGG AGAAGCCTTT ATTTCAACGC |
| 2461 | AAGGATAAAA ATTTTTAGAA CCCTCATATA TTTTAAATGC AATGCCTGAG TAATGTGTAG |
| 2521 | GTAAAGATTC AAACGGGTGA GAAAGGCCGG AGACAGTCAA ATCACCATCA ATATGATATT |
| 2581 | CAACCGTTCT AGCTGATAAA TTCATGCCGG AGAGGGTAGC TATTTTTGAG AGGTCTCTAC |
| 2641 | AAAGGCTATC AGGTCATTGC CTGAGAGTCT GGAGCAAACA AGAGAATCGA TGAACGGTAA |
| 2701 | TCGTAAAACT AGCATGTCAA TCATATGTAC CCCGGTTGAT AATCAGAAAA GCCCCAAAAA |
| 2761 | CAGGAAGATT GTATAAGCAA ATATTTAAAT TGTAAGCGTT AATATTTTGT TAAAATTCGC |
| 2821 | GTTAAATTTT TGTTAAATCA GCTCATTTTT TAACCAATAG GCCGAAATCG GCAAAATCCC |
| 2881 | TTATAAATCA AAAGAATAGA CCGAGATAGG GTTGAGTGTT GTTCCAGTTT GGAACAAGAG |
| 2941 | TCCACTATTA AAGAACGTGG ACTCCAACGT CAAAGGGCGA AAAACCGTCT ATCAGGGCGA |
| 3001 | TGGCCCACTA CGTGAACCAT CACCCTAATC AAGTTTTTTG GGGTCGAGGT GCCGTAAAGC |
| 3061 | ACTAAATCGG AACCCTAAAG GGAGCCCCCG ATTTAGAGCT TGACGGGGAA AGCCGGCGAA |
| 3121 | CGTGGCGAGA AAGGAAGGGA AGAAAGCGAA AGGAGCGGGC GCTAGGGCGC TGGCAAGTGT |
| 3181 | AGCGGTCACG CTGCGCGTAA CCACCACACC CGCCGCGCTT AATGCGCCGC TACAGGGCGC |
| 3241 | GTACTATGGT TGCTTTGACG AGCACGTATA ACGTGCTTTC CTCGTTAGAA TCAGAGCGGG |
| 3301 | AGCTAAACAG GAGGCCGATT AAAGGGATTT TAGACAGGAA CGGTACGCCA GAATCCTGAG |
| 3361 | AAGTGTTTTT ATAATCAGTG AGGCCACCGA GTAAAAGAGT CTGTCCATCA CGCAAATTAA |
| 3421 | CCGTTGTCGC AATACTTCTT TGATTAGTAA TAACATCACT TGCCTGAGTA GAAGAACTCA |
| 3481 | AACTATCGGC CTTGCTGGTA ATATCCAGAA CAATATTACC GCCAGCCATT GCAACGGAAT |
| 3541 | CGCCATTCGC CATTCAGGCT GCGCAACTGT TGGGAAGGGC GATCGGTGCG GGCCTCTTCC |
| 3601 | ACTGAGGCCC AGCTGCGCGC TCGCTCGCTC ACTGAGGCCG CCCGGGCAAA GCCCGGGCGT |
| 3661 | CGGGCGACCT TTGGTCGCCC GGCCTCAGTG AGCGAGCGAG CGCGCAGAGA GGGAGTGGCC |
| 3721 | AACTCCATCA CTAGGGGTTC CTTGTAGTTA ATGATTAACC CGCCATGCTA CTTATCTACT |
| 3781 | CGACATTGAT TATTGACTAG TTATTAATAG TAATCAATTA CGGGGTCATT AGTTCATAGC |
| 3841 | CCATATATGG AGTTCCGCGT TACATAACTT ACGGTAAATG GCCCGCCTGG CTGACCGCCC |
| 3901 | AACGACCCCC GCCCATTGAC GTCAATAATG ACGTATGTTC CCATAGTAAC GCCAATAGGG |
| 3961 | ACTTTCCATT GACGTCAATG GGTGGAGTAT TTACGGTAAA CTGCCCACTT GGCAGTACAT |
| 4021 | CAAGTGTATC ATATGCCAAG TACGCCCCCT ATTGACGTCA ATGACGGTAA ATGGCCCGCC |
| 4081 | TGGCATTATG CCCAGTACAT GACCTTATGG GACTTTCCTA CTTGGCAGTA CATCTACGTA |
| 4141 | TTAGTCATCG CTATTACCAT GGTCGAGGTG AGCCCCACGT TCTGCTTCAC TCTCCCCATC |
| 4201 | TCCCCCCCCT CCCCACCCCC AATTTTGTAT TTATTTATTT TTTAATTATT TTGTGCAGCG |
| 4261 | ATGGGGGCGG GGGGGGGGGG GGGGCGCGCG CCAGGCGGGG CGGGGCGGGG CGAGGGGCGG |
| 4321 | GGCGGGGCGA GGCGGAGAGG TGCGGCGGCA GCCAATCAGA GCGGCGCGCT CCGAAAGTTT |
| 4381 | CCTTTTATGG CGAGGCGGCG GCGGCGGCGG CCCTATAAAA AGCGAAGCGC GCGGCGGGCG |
| 4441 | GGAGTCGCTG CGTTGCCTTC GCCCCGTGCC CCGCTCCGCG CCGCCTCGCG CCGCCCGCCC |
| 4501 | CGGCTCTGAC TGACCGCGTT ACTCCCACAG GTGAGCGGGC GGGACGGCCC TTCTCCTCCG |
| 4561 | GGCTGTAATT AGCGCTTGGT TTAATGACGG CTTGTTTCTT TTCTGTGGCT GCGTGAAAGC |
| 4621 | CTTGAGGGGC TCCGGGAGGG CCCTTTGTGC GGGGGGAGCG GCTCGGGGGG TGCGTGCGTG |
| 4681 | TGTGTGTGCG TGGGGAGCGC CGCGTGCGGC TCCGCGCTGC CCGGCGGCTG TGAGCGCTGC |
| 4741 | GGGCGCGGCG CGGGGCTTTG TGCGCTCCGC AGTGTGCGCG AGGGGAGCGC GGCCGGGGGC |
| 4801 | GGTGCCCCGC GGTGCGGGGG GCTGCGAGGG GAACAAAGGC TGCGTGCGGG GTGTGTGCGT |
| 4861 | GGGGGGGTGA GCAGGGGGTG TGGGCGCGTC GGTCGGGCTG CAACCCCCCC TGCACCCCCC |
| 4921 | TCCCCGAGTT GCTGAGCACG GCCCGGCTTC GGGTGCGGGG CTCCGTACGG GGCGTGGCGC |
| 4981 | GGGGCTCGCC GTGCCGGGCG GGGGGTGGCG GCAGGTGGGG GTGCCGGGCG GGGCGGGGCC |
| 5041 | GCCTCGGGCC GGGGAGGGCT CGGGGGAGGG GCGCGGCGGC CCCCGGAGCG CCGGCGGCTG |
| 5101 | TCGAGGCGCG GCGAGCCGCA GCCATTGCCT TTTATGGTAA TCGTGCGAGA GGGCGCAGGG |
| 5161 | ACTTCCTTTG TCCCAAATCT GTGCGGAGCC GAAATCTGGG AGGCGCCGCC GCACCCCCTC |
| 5221 | TAGCGGGCGC GGGGCGAAGC GGTGCGGCGC CGGCAGGAAG GAAATGGGCG GGGAGGGCCT |
| 5281 | TCGTGCGTCG CCGCGCCGCC GTCCCCTTCT CCCTCTCCAG CCTCGGGGCT GTCCGCGGGG |
| 5341 | GGACGGCTGC CTTCGGGGGG GACGGGGCAG GGCGGGGTTC GGCTTCTGGC GTGTGACCGG |
| 5401 | CGGCTCTAGA GCCTCTGCTA ACCATGTTCA TGCCTTCTTC TTTTTCCTAC AGCTCCTGGG |
| 5461 | CAACGTGCTG GTTATTGTGC TGTCTCATCA TTTTGGCAAA GAATTGATTA ATTCGAGCGA |
| 5521 | ACGCGTCGAG TCGCTCGGTA CGATTTAAAT TGAATTGGCC TCGAGCGCAA GCTTGAGCTA |
| 5581 | GCGCCACCAT GGAATGGATG AGAAGCAGAG TGGGCACCCT GGGCCTGTGG GTGCGACTGC |
| 5641 | TGCTGGCTGT GTTTCTGCTG GGCGTGTACC AGGCCTACCC CATCCCTGAC TCTAGCCCCC |
| 5701 | TGCTGCAGTT TGGCGGACAA GTGCGGCAGA GATACCTGTA CACCGACGAC GACCAGGACA |
| 5761 | CCGAGGCCCA CCTGGAAATC CGCGAGGATG GCACAGTCGT GGGCGCTGCT CACAGAAGCC |
| 5821 | CTGAGAGCCT GCTGGAACTG AAGGCCCTGA AGCCCGGCGT GATCCAGATC CTGGGCGTGA |
| 5881 | AGGCCAGCAG ATTCCTGTGC CAGCAGCCTG ACGGCGCCCT GTACGGCTCT CCTCACTTCG |
| 5941 | ATCCTGAGGC CTGCAGCTTC AGAGAGCTGC TGCTGGAGGA CGGCTACAAC GTGTACCAGT |
| 6001 | CTGAGGCCCA CGGCCTGCCC CTGAGACTGC CTCAGAAGGA CAGCCCTAAC CAGGACGCCA |
| 6061 | CAAGCTGGGG ACCTGTGCGG TTCCTGCCTA TGCCTGGACT GCTGCACGAG CCCCAGGATC |
| 6121 | AGGCTGGCTT TCTGCCTCCT GAGCCTCCAG ACGTGGGCAG CAGCGACCCT CTGAGCATGG |
| 6181 | TGGAACCTCT GCAGGGCAGA AGCCCCAGCT ACGCCTCTTG AGAATGCGGG CCCGGTACCC |
| 6241 | CCGACGCGGC CTAACTGGCC TCATGGGCCT TCCGCTCACT GCCCGCTTTC CAGTCGGGAA |
| 6301 | ACCTGTCGTG CCAGTCAGGT GCAGGCTGCC TATCAGAAGG TGGTGGCTGG TGTGGCCAAT |
| 6361 | GCCCTGGCTC ACAAATACCA CTGAGATCTT TTTCCCTCTG CCAAAAATTA TGGGGACATC |
| 6421 | ATGAAGCCCC TTGAGCATCT GACTTCTGGC TAATAAAGGA AATTTATTTT CATTGCAATA |
| 6481 | GTGTGTTGGA ATTTTTTGTG TCTCTCACTC GGAAGGACAT ATGGGAGGGC AAATCATTTA |
| 6541 | AAACATCAGA ATGAGTATTT GGTTTAGAGT TTGGCAACAT ATGCCCATAT GCTGGCTGCC |
| 6601 | ATGAACAAAG GTTGGCTATA AAGAGGTCAT CAGTATATGA AACAGCCCCC TGCTGTCCAT |
| 6661 | TCCTTATTCC ATAGAAAAGC CTTGACTTGA GGTTAGATTT TTTTTATATT TTGTTTTGTG |
| 6721 | TTATTTTTTT CTTTAACATC CCTAAAATTT TCCTTACATG TTTTACTAGC CAGATTTTTC |
| 6781 | CTCCTCTCCT GACTACTCCC AGTCATAGCT GTCCCTCTTC TCTTATGGAG ATCCCTCGAC |
| 6841 | CTGCAGCCCA AGCTGTAGAT AAGTAGCATG GCGGGTTAAT CATTAACTAC AAGGAACCCC |
| 6901 | TAGTGATGGA GTTGGCCACT CCCTCTCTGC GCGCTCGCTC GCTCACTGAG GCCGGGCGAC |
| 6961 | CAAAGGTCGC CCGACGCCCG GGCTTTGCCC GGGCGGCCTC AGTGAGCGAG CGAGCGCGCA |
| 7021 | GCTGGCGTAA |

AAV2 5′ ITR: 3601-3742 bp
CAG promoter: 3779-5423 bp
Mus musculus codon-optimized FGF21 (moFGF21): 5588-6221 bp
Rabbit β-globin polyA signal (3′ UTR and 3′ flanking region of rabbit beta-globin,
including polyA signal): 6315-6833 bp
AAV2 3′ ITR: 6892-7024 bp
pAAV-CMV-moFGF21 (SEQ ID NO: 63)

| | |
|---|---|
| 1 | GGGGCTAGCG CCACCATGGA ATGGATGAGA AGCAGAGTGG GCACCCTGGG |
| 51 | CCTGTGGGTG CGACTGCTGC TGGCTGTGTT TCTGCTGGGC GTGTACCAGG |
| 101 | CCTACCCCAT CCCTGACTCT AGCCCCCTGC TGCAGTTTGG CGGACAAGTG |
| 151 | CGGCAGAGAT ACCTGTACAC CGACGACGAC CAGGACACCG AGGCCCACCT |
| 201 | GGAAATCCGC GAGGATGGCA CAGTCGTGGG CGCTGCTCAC AGAAGCCCTG |
| 251 | AGAGCCTGCT GGAACTGAAG GCCCTGAAGC CCGGCGTGAT CCAGATCCTG |
| 301 | GGCGTGAAGG CCAGCAGATT CCTGTGCCAG CAGCCTGACG GCGCCCTGTA |
| 351 | CGGCTCTCCT CACTTCGATC CTGAGGCCTG CAGCTTCAGA GAGCTGCTGC |
| 401 | TGGAGGACGG CTACAACGTG TACCAGTCTG AGGCCCACGG CCTGCCCCTG |
| 451 | AGACTGCCTC AGAAGGACAG CCCTAACCAG GACGCCACAA GCTGGGGACC |
| 501 | TGTGCGGTTC CTGCCTATGC CTGGACTGCT GCACGAGCCC CAGGATCAGG |
| 551 | CTGGCTTTCT GCCTCCTGAG CCTCCAGACG TGGGCAGCAG CGACCCTCTG |
| 601 | AGCATGGTGG AACCTCTGCA GGGCAGAAGC CCCAGCTACG CCTCTTGAGA |
| 651 | ATGCGGGCCC GGTACCCCCT CGACGGTACC AGCGCTGTCG AGGCCGCTTC |
| 701 | GAGCAGACAT GATAAGATAC ATTGATGAGT TTGGACAAAC CACAACTAGA |
| 751 | ATGCAGTGAA AAAAATGCTT TATTTGTGAA ATTTGTGATG CTATTGCTTT |
| 801 | ATTTGTAACC ATTATAAGCT GCAATAAACA AGTTAACAAC AACAATTGCA |
| 851 | TTCATTTTAT GTTTCAGGTT CAGGGGGAGA TGTGGGAGGT TTTTTAAAGC |
| 901 | AAGTAAAACC TCTACAAATG TGGTAAAATC GATTAGGATC TTCCTAGAGC |
| 951 | ATGGCTACCT AGACATGGCT CGACAGATCA GCGCTCATGC TCTGGAAGAT |
| 1001 | CTCGATTTAA ATGCGGCCGC AGGAACCCCT AGTGATGGAG TTGGCCACTC |
| 1051 | CCTCTCTGCG CGCTCGCTCG CTCACTGAGG CCGGGCGACC AAAGGTCGCC |
| 1101 | CGACGCCCGG GCTTTGCCCG GGCGGCCTCA GTGAGCGAGC GAGCGCGCAG |
| 1151 | CTGCCTGCAG GGGCGCCTGA TGCGGTATTT TCTCCTTACG CATCTGTGCG |
| 1201 | GTATTTCACA CCGCATACGT CAAAGCAACC ATAGTACGCG CCCTGTAGCG |
| 1251 | GCGCATTAAG CGCGGCGGGT GTGGTGGTTA CGCGCAGCGT GACCGCTACA |
| 1301 | CTTGCCAGCG CCCTAGCGCC CGCTCCTTTC GCTTTCTTCC CTTCCTTTCT |
| 1351 | CGCCACGTTC GCCGGCTTTC CCCGTCAAGC TCTAAATCGG GGGCTCCCTT |
| 1401 | TAGGGTTCCG ATTTAGTGCT TTACGGCACC TCGACCCCAA AAAACTTGAT |
| 1451 | TTGGGTGATG GTTCACGTAG TGGGCCATCG CCCTGATAGA CGGTTTTTCG |
| 1501 | CCCTTTGACG TTGGAGTCCA CGTTCTTTAA TAGTGGACTC TTGTTCCAAA |
| 1551 | CTGGAACAAC ACTCAACCCT ATCTCGGGCT ATTCTTTTGA TTTATAAGGG |
| 1601 | ATTTTGCCGA TTTCGGCCTA TTGGTTAAAA AATGAGCTGA TTTAACAAAA |
| 1651 | ATTTAACGCG AATTTTAACA AAATATTAAC GTTTACAATT TTATGGTGCA |
| 1701 | CTCTCAGTAC AATCTGCTCT GATGCCGCAT AGTTAAGCCA GCCCCGACAC |
| 1751 | CCGCCAACAC CCGCTGACGC GCCCTGACGG GCTTGTCTGC TCCCGGCATC |
| 1801 | CGCTTACAGA CAAGCTGTGA CCGTCTCCGG GAGCTGCATG TGTCAGAGGT |
| 1851 | TTTCACCGTC ATCACCGAAA CGCGCGAGAC GAAAGGGCCT CGTGATACGC |
| 1901 | CTATTTTTAT AGGTTAATGT CATGATAATA ATGGTTTCTT AGACGTCAGG |
| 1951 | TGGCACTTTT CGGGGAAATG TGCGCGGAAC CCCTATTTGT TTATTTTTCT |
| 2001 | AAATACATTC AAATATGTAT CCGCTCATGA GACAATAACC CTGATAAATG |
| 2051 | CTTCAATAAT ATTGAAAAAG GAAGAGTATG AGTATTCAAC ATTTCCGTGT |
| 2101 | CGCCCTTATT CCCTTTTTTG CGGCATTTTG CCTTCCTGTT TTTGCTCACC |
| 2151 | CAGAAACGCT GGTGAAAGTA AAAGATGCTG AAGATCAGTT GGGTGCACGA |
| 2201 | GTGGGTTACA TCGAACTGGA TCTCAACAGC GGTAAGATCC TTGAGAGTTT |
| 2251 | TCGCCCCGAA GAACGTTTTC CAATGATGAG CACTTTTAAA GTTCTGCTAT |
| 2301 | GTGGCGCGGT ATTATCCCGT ATTGACGCCG GGCAAGAGCA ACTCGGTCGC |
| 2351 | CGCATACACT ATTCTCAGAA TGACTTGGTT GAGTACTCAC CAGTCACAGA |
| 2401 | AAAGCATCTT ACGGATGGCA TGACAGTAAG AGAATTATGC AGTGCTGCCA |
| 2451 | TAACCATGAG TGATAACACT GCGGCCAACT TACTTCTGAC AACGATCGGA |
| 2501 | GGACCGAAGG AGCTAACCGC TTTTTTGCAC AACATGGGGG ATCATGTAAC |
| 2551 | TCGCCTTGAT CGTTGGGAAC CGGAGCTGAA TGAAGCCATA CCAAACGACG |
| 2601 | AGCGTGACAC CACGATGCCT GTAGCAATGG CAACAACGTT GCGCAAACTA |
| 2651 | TTAACTGGCG AACTACTTAC TCTAGCTTCC CGGCAACAAT TAATAGACTG |
| 2701 | GATGGAGGCG GATAAAGTTG CAGGACCACT TCTGCGCTCG GCCCTTCCGG |
| 2751 | CTGGCTGGTT TATTGCTGAT AAATCTGGAG CCGGTGAGCG TGGGTCTCGC |
| 2801 | GGTATCATTG CAGCACTGGG GCCAGATGGT AAGCCCTCCC GTATCGTAGT |
| 2851 | TATCTACACG ACGGGGAGTC AGGCAACTAT GGATGAACGA AATAGACAGA |
| 2901 | TCGCTGAGAT AGGTGCCTCA CTGATTAAGC ATTGGTAACT GTCAGACCAA |
| 2951 | GTTTACTCAT ATATACTTTA GATTGATTTA AAACTTCATT TTTAATTTAA |
| 3001 | AAGGATCTAG GTGAAGATCC TTTTTGATAA TCTCATGACC AAAATCCCTT |
| 3051 | AACGTGAGTT TTCGTTCCAC TGAGCGTCAG ACCCCGTAGA AAAGATCAAA |
| 3101 | GGATCTTCTT GAGATCCTTT TTTTCTGCGC GTAATCTGCT GCTTGCAAAC |
| 3151 | AAAAAAACCA CCGCTACCAG CGGTGGTTTG TTTGCCGGAT CAAGAGCTAC |
| 3201 | CAACTCTTTT TCCGAAGGTA ACTGGCTTCA GCAGAGCGCA GATACCAAAT |
| 3251 | ACTGTCCTTC TAGTGTAGCC GTAGTTAGGC CACCACTTCA AGAACTCTGT |
| 3301 | AGCACCGCCT ACATACCTCG CTCTGCTAAT CCTGTTACCA GTGGCTGCTG |
| 3351 | CCAGTGGCGA TAAGTCGTGT CTTACCGGGT TGGACTCAAG ACGATAGTTA |
| 3401 | CCGGATAAGG CGCAGCGGTC GGGCTGAACG GGGGGTTCGT GCACACAGCC |
| 3451 | CAGCTTGGAG CGAACGACCT ACACCGAACT GAGATACCTA CAGCGTGAGC |
| 3501 | TATGAGAAAG CGCCACGCTT CCCGAAGGGA GAAAGGCGGA CAGGTATCCG |
| 3551 | GTAAGCGGCA GGGTCGGAAC AGGAGAGCGC ACGAGGGAGC TTCCAGGGGG |
| 3601 | AAACGCCTGG TATCTTTATA GTCCTGTCGG GTTTCGCCAC CTCTGACTTG |
| 3651 | AGCGTCGATT TTTGTGATGC TCGTCAGGGG GGCGGAGCCT ATGGAAAAAC |
| 3701 | GCCAGCAACG CGGCCTTTTT ACGGTTCCTG GCCTTTTGCT GGCCTTTTGC |
| 3751 | TCACATGTCC TGCAGGCAGC TGCGCGCTCG CTCGCTCACT GAGGCCGCCC |
| 3801 | GGGCAAAGCC CGGGCGTCGG GCGACCTTTG GTCGCCCGGC CTCAGTGAGC |
| 3851 | GAGCGAGCGC GCAGAGAGGG AGTGGCCAAC TCCATCACTA GGGGTTCCTG |
| 3901 | CGGCCGCGAT ATCTGTAGTT AATGATTAAC CCGCCATGCT ACTTATCTAC |
| 3951 | AGATCTCAAT ATTGGCCATT AGCCATATTA TTCATTGGTT ATATAGCATA |
| 4001 | AATCAATATT GGCTATTGGC CATTGCATAC GTTGTATCTA TATCATAATA |
| 4051 | TGTACATTTA TATTGGCTCA TGTCCAATAT GACCGCCATG TTGGCATTGA |
| 4101 | TTATTGACTA GTTATTAATA GTAATCAATT ACGGGGTCAT TAGTTCATAG |
| 4151 | CCCATATATG GAGTTCCGCG TTACATAACT TACGGTAAAT GGCCCGCCTG |
| 4201 | GCTGACCGCC CAACGACCCC CGCCCATTGA CGTCAATAAT GACGTATGTT |
| 4251 | CCCATAGTAA CGCCAATAGG GACTTTCCAT TGACGTCAAT GGGTGGAGTA |
| 4301 | TTTACGGTAA ACTGCCCACT TGGCAGTACA TCAAGTGTAT CATATGCCAA |
| 4351 | GTCCGCCCCC TATTGACGTC AATGACGGTA AATGGCCCGC CTGGCATTAT |
| 4401 | GCCCAGTACA TGACCTTACG GGACTTTCCT ACTTGGCAGT ACATCTACGT |
| 4451 | ATTAGTCATC GCTATTACCA TGGTGATGCG GTTTTGGCAG TACACCAATG |
| 4501 | GGCGTGGATA GCGGTTTGAC TCACGGGGAT TTCCAAGTCT CCACCCCATT |
| 4551 | GACGTCAATG GGAGTTTGTT TTGGCACCAA AATCAACGGG ACTTTCCAAA |
| 4601 | ATGTCGTAAC AACTGCGATC GCCCGCCCCG TTGACGCAAA TGGGCGGTAG |
| 4651 | GCGTGTACGG TGGGAGGTCT ATATAAGCAG AGCTCGTTTA GTGAACCGTC |
| 4701 | AGATCACTAG GCTAGCTATT GCGGTAGTTT ATCACAGTTA AATTGCTAAC |
| 4751 | GCAGTCAGTG CTTCTGACAC AACAGTCTCG AACTTAAGCT GCAGTGACTC |
| 4801 | TCTTAAGGTA GCCTTGCAGA AGTTGGTCGT GAGGCACTGG GCAGGTAAGT |
| 4851 | ATCAAGGTTA CAAGACAGGT TTAAGGAGAC CAATAGAAAC TGGGCTTGTC |
| 4901 | GAGACAGAGA AGACTCTTGC GTTTCTGATA GGCACCTATT GGTCTTACTG |
| 4951 | ACATCCACTT TGCCTTTCTC TCCACAGGTG TCCACTCCCA GTTCAATTAC |
| 5001 | AGCTCTTAAG GCTAGAGTAC TTAATACGAC TCACTATAGA ATACGACTCA |
| 5051 | CTATAGGGAG ACGCTAGCGT CGA |

AAV2 5′ ITR: 3772-3899 bp
CMV enhancer: 4093-4472 bp
CMV promoter: 4473-4684 bp
β-globin intron (chimeric intron composed of introns from human β-globin
and immunoglobulin heavy chain genes): 4845-4977 bp
Mus musculus codon-optimized FGF21 (moFGF21): 16-648 bp
SV40 polyA signal: 713-834 bp
AAV2 3′ ITR: 1021-1148 bp

1. A method for the treatment and/or prevention of a central nervous system (CNS) disorder or disease, the method comprising administering a gene construct comprising a nucleotide sequence encoding a fibroblast growth factor 21 (FGF21).

2. The method according to claim 1, wherein the nucleotide sequence encoding FGF21 is operably linked to a ubiquitous promoter, preferably wherein the ubiquitous promoter is selected from the group consisting of a CAG promoter and a CMV promoter.

3. The method according to claim 1, wherein the gene construct comprises at least one target sequence of a microRNA expressed in a tissue where the expression of FGF21 is wanted to be prevented, preferably wherein the at least one target sequence of a microRNA is selected from those target sequences that bind to microRNAs expressed in heart and/or liver of a mammal.

4. The method according to claim 3, wherein the gene construct comprises at least one target sequence of a microRNA expressed in the liver and at least one target sequence of a microRNA expressed in the heart, preferably wherein a target sequence of a microRNA expressed in the heart is selected from SEQ ID NO's: 13 and 21-25 and a target sequence of a microRNA expressed in the liver is selected from SEQ ID NO's: 12 and 14-20, more preferably wherein the gene construct comprises a target sequence of microRNA-122a (SEQ ID NO: 12) and a target sequence of microRNA-1 (SEQ ID NO: 13).

5. The method according to claim 1, wherein the nucleotide sequence encoding FGF21 is selected from the group consisting of:
(a) a nucleotide sequence encoding a polypeptide represented by an amino acid sequence comprising a sequence that has at least 60% sequence identity or similarity with the amino acid sequence of SEQ ID NO: 1, 2 or 3;
(b) a nucleotide sequence that has at least 60% sequence identity with the nucleotide sequence of SEQ ID NO: 4, 5, 6, 7, 8, 9, 10 or 11; and
(c) a nucleotide sequence the sequence of which differs from the sequence of a nucleotide sequence of (b) due to the degeneracy of the genetic code.

6. The method according to claim 16, wherein the expression vector is a viral vector, more preferably wherein the expression vector is selected from the group consisting of adenoviral vectors, adeno-associated viral vectors, retroviral vectors, and lentiviral vectors.

7. The method according to claim 6, wherein the expression vector is an adeno-associated viral vector, preferably an adeno-associated viral vector of serotype 1, 2, 3, 4, 5, 6, 7, 8, 9, rh10, rh8, Cb4, rh74, DJ, 2/5, 2/1, 1/2 or Anc80, more preferably an adeno-associated viral vector of serotype 1, 8 or 9.

8. The method according to claim 1, wherein the gene construct is comprised in a pharmaceutical composition.

9. The method according to claim 1, wherein the central nervous system (CNS) disorder or disease is associated with and/or caused by aging and/or a metabolic disorder or disease, preferably obesity and/or diabetes.

10. The method according to claim 1, wherein the central nervous system (CNS) disorder or disease is neuroinflammation, neurodegeneration, cognitive decline and/or a disease or condition associated therewith.

11. The method according to claim 10, wherein the disease or condition associated with and/or caused by neuroinflammation, neurodegeneration and/or cognitive decline is selected from the group consisting of: a cognitive disorder, dementia, Alzheimer's disease, vascular dementia, Lewy body dementia, frontotemporal dementia (FTD), Parkinson's disease, Parkinson-like disease, Parkinsonism, Huntington's disease, traumatic brain injury, prion disease, dementia/neurocognitive issues due to HIV infection, dementia/neurocognitive issues due to aging, tauopathy, multiple sclerosis and other neuroinflammatory/neurodegenerative diseases, preferably selected from the group consisting of Alzheimer's disease, Parkinson's disease, Parkinson-like disease and Huntington's disease, more preferably selected from the group consisting of Alzheimer's disease and Parkinson's disease, most preferably Alzheimer's disease.

12. The method according to claim 1, wherein the central nervous system (CNS) disorder or disease is a behavioral disorder, preferably an anxiety disorder or a depressive disorder.

13. The method according to claim 1, wherein the central nervous system (CNS) disorder or disease is a neuromuscular disorder, preferably wherein the neuromuscular disorder is, or is associated with, declined muscle function, declined muscle strength, declined coordination, declined balance and/or hypoactivity.

14. A method for improving memory and/or learning in a subject, the method comprising administering to the subject a gene construct comprising a nucleotide sequence encoding a fibroblast growth factor 21 (FG21).

15. A method for improving muscle function, muscle strength, coordination, balance and/or hypoactivity in a subject, the method comprising administering to the subject a gene construct comprising a nucleotide sequence encoding a fibroblast growth factor 21 (FG21).

16. The method of claim 1, wherein the gene construct is comprised in an expression vector.

17. The method of claim 16, wherein the expression vector is comprised in a pharmaceutical composition.

18. The method of claim 14, wherein the subject is an elderly subject and/or a subject diagnosed with a metabolic disorder or disease, preferably diabetes and/or obesity.

19. The method of claim 15, wherein the subject is an elderly subject and/or a subject diagnosed with a metabolic disorder or disease, preferably diabetes and/or obesity.
